(19) United States
(12) Patent Application Publication
(10) Pub. No.: US 2009/0023704 A1
(43) Pub. Date: Jan. 22, 2009

(54) Novel Compounds 737

(75) Inventors: Leifeng Cheng, Molndal (SE); Sara Holmqvist, Molndal (SE); Florian Raubacher, Molndal (SE); Peter Schell, Molndal (SE)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 12/104,469

(22) Filed: Apr. 17, 2008

Related U.S. Application Data
(60) Provisional application No. 60/912,533, filed on Apr. 18, 2007.
(60) Provisional application No. 60/940,474, filed on May 29, 2007.

(51) Int. Cl. C07D 473/04; A61K 31/522; A61P 1/00

(52) U.S. Cl. 514/210.21; 544/267; 514/263.34; 514/263.22; 544/269; 514/252.16; 514/228.5; 544/61; 544/118; 514/234.2

(57) ABSTRACT

The present invention relates to novel xanthine compounds of the general formula (I)

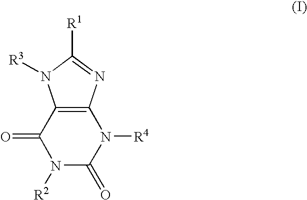

wherein R¹, R², R³ and R⁴ are as defined,
having a positive allosteric GABA_{B} receptor (GBR) modulator effect, methods for the preparation of said compounds and to their use, optionally in combination with a GABA_{B} agonist, for the inhibition of transient lower esophageal sphincter relaxations, for the treatment of gastroesophageal reflux disease, as well as for the treatment of functional gastrointestinal disorders and irritable bowel syndrome (IBS).

### FIELD OF THE INVENTION

The present invention relates to novel xanthine compounds having a positive allosteric GABA_{B} receptor (GBR) modulator effect, methods for the preparation of said compounds and their use for the inhibition of transient lower esophageal sphincter relaxations, for the treatment of gastroesophageal reflux disease, as well as for the treatment of functional gastrointestinal disorders and irritable bowel syndrome (IBS).

### BACKGROUND OF THE INVENTION

The lower esophageal sphincter (LES) is prone to relaxing intermittently. As a consequence, fluid from the stomach can pass into the esophagus since the mechanical barrier (the esophageal sphincter) is temporarily not functioning as desired at such times. Such as a condition is hereinafter referred to as “reflux”.

Gastroesophageal reflux disease (GERD) is the most prevalent upper gastrointestinal tract disease. Current pharmacotherapy aims at reducing gastric acid secretion, or at neutralizing acid in the esophagus. The major mechanism behind reflux has been considered to depend on a hypotonic lower esophageal sphincter. However, recent research (e.g. Holloway & Dent (1990) Gastroenterol. Clin. N. Amer. 19, pp. 517-535) has shown that most reflux episodes occur during transient lower esophageal sphincter relaxations (TLESR), i.e. relaxations not triggered by swallows. It has also been shown that gastric acid secretion usually is normal in patients with GERD.

Consequently, there is a need for a therapy that reduces the incidence of TLESR and thereby prevents reflux.

GABA_{B}-receptor agonists have been shown to inhibit TLESR, which is disclosed in WO 98/11885 A1.

Functional gastrointestinal disorders, such as functional dyspepsia, can be defined in accordance with Thompson W G, Longstreth G F, Drossman D A, Heaton K W, Irvine E J, Mueller-Lissner S A. C. Functional Bowel Disorders and Functional Abdominal Pain. In: Drossman D A, Talley N J, Thompson W G, Whitehead W E, Coraziarri E, eds. Rome II: Functional Gastrointestinal Disorders: Diagnosis, Pathophysiology and Treatment. 2 ed. McLean, V A: Degnon Associates, Inc.; 2000.351-432 and Drossman D A, Corazziari E, Talley N J, Thompson W G and Whitehead W E. Rome II: A multinational consensus document on Functional Gastrointestinal Disorders. Gut 45(Suppl. 2), II1-II81.9-1-1999.

Irritable bowel syndrome (IBS) can be defined in accordance with Thompson W G, Longstreth G F, Drossman D A, Heaton K W, Irvine E J, Mueller-Lissner S A. C. Functional Bowel Disorders and Functional Abdominal Pain. In: Drossman D A, Talley N J, Thompson W G, Whitehead W E, Coraziarri E, eds. Rome II: Functional Gastrointestinal Disorders: Diagnosis, Pathophysiology and Treatment. 2 ed. McLean, V A: Degnon Associates, Inc.; 2000:351-432 and Drossman D A, Corazziari E, Talley N J, Thompson W G and Whitehead W E. Rome II: A multinational consensus document on Functional Gastrointestinal Disorders. Gut 45(Suppl. 2), II1-II81.9-1-1999.

### GABA_{B} Receptor Agonists

GABA (4-aminobutanoic acid) is an endogenous neurotransmitter in the central and peripheral nervous systems. Receptors for GABA have traditionally been divided into GABA_{A} and GABA_{B} receptor subtypes. GABA_{B} receptors belong to the superfamily of G-protein coupled receptors (GPCRs).

The most studied GABA_{B} receptor agonist baclofen (4-amino-3-(p-chlorophenyl)butanoic acid; disclosed in CH 449046) is useful as an antispastic agent. EP 356128 A2 describes the use of the GABA_{B} receptor agonist (3-aminopropyl)methylphosphinic acid for use in therapy, in particular in the treatment of central nervous system disorders.

EP 463969 A1 and FR 2722192 A1 disclose 4-aminobutanoic acid derivatives having different heterocyclic substituents at the 3-carbon of the butyl chain. EP 181833 A1 discloses substituted 3-aminopropylphosphinic acids having high affinities towards GABA_{B} receptor sites. EP 399949 A1 discloses derivatives of (3-aminopropyl)methylphosphinic acid, which are described as potent GABA_{B} receptor agonists. Still other (3-aminopropyl)methylphosphinic acids and (3-aminopropyl)phosphinic acids have been disclosed in WO 01/41743 A1 and WO 01/42252 A1, respectively. Structure-activity relationships of several phosphinic acid analogues with respect to their affinities to the GABA_{B} receptor are discussed in J. Med. Chem. (1995), 38, 3297-3312. Sulphinic acid analogues and their GABA_{B} receptor activities are described in Bioorg. & Med. Chem. Lett. (1998), 8, 3059-3064. For a more general review on GABA_{B} ligands, see Curr. Med. Chem.-Central Nervous System Agents (2001), 1, 27-42.

### Positive Allosteric Modulation of GABA_{B} Receptors

2,6-Di-tert-butyl-4-(3-hydroxy-2,2-dimethylpropyl)phenol (CGP7930) and 3-(3,5-di-tert-butyl-4-hydroxyphenyl)-2,2-dimethylpropanal (disclosed in U.S. Pat. No. 5,304,685) have been described to exert positive allosteric modulation of native and recombinant GABA_{B} receptor activity (Society for Neuroscience, 30^{th} Annual Meeting, New Orleans, La., Nov. 4-9, 2000: Positive Allosteric Modulation of Native and Recombinant GABA_{B} Receptor Activity, S. Urwyler et al.; Molecular Pharmacol. (2001), 60, 963-971).

N,N-Dicyclopentyl-2-methylsulfanyl-5-nitro-pyrimidine-4,6-diamine has been described to exert positive allosteric modulation of the GABA_{B} receptor (The Journal of Pharmacology and Experimental Therapeutics, 307 (2003), 322-330).

### Xanthine Derivatives

WO 9618400 discloses 1-(4-chlorobenzyl)-3-ethyl-8-isopropyl-xanthine as an intermediate in the preparation of Example 3 and 1,3-di-(4-chlorobenzyl)-8-isopropyl-xanthine as an intermediate in the preparation of Example 10.

WO 8601724 discloses 1,3-dibenzylxanthine as a pest control agent.

WO 9107945 discloses 1-benzyl-3-isobutylxanthine as an agent to help the pigmentation of skin or hair.

WO 9502604 discloses 1,3-dibenzylxanthine as an A3 adenosine receptor agonist and 1-benzyl-3-butylxanthine as a starting compound for the preparation of Example 61.

### OUTLINE OF THE INVENTION

The present invention provides a compound of the general formula (I)

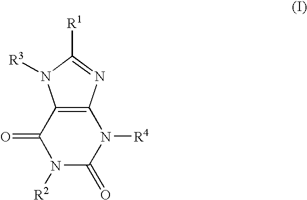

as well as pharmaceutically acceptable salts thereof;
wherein
R¹ is selected from halogen; C₁-C₁₀ alkyl; C₁-C₁₀ alkoxy; hydroxy-C₁-C₁₀ alkyl; C₁-C₁₀ alkoxy-C₁-C₁₀ alkyl; C₃-C₁₀ cycloalkyl; amino substituted by one or more of C₁-C₁₀ alkyl and C₁-C₁₀ alkoxy-C₁-C₁₀ alkyl; and heterocyclyl unsubstituted or substituted by one or more of C₁-C₁₀ alkyl, C₁-C₁₀ alkoxy, C₁-C₁₀ alkoxy-C₁-C₁₀ alkyl, di-C₁-C₁₀ alkylamino, oxo and heterocyclyl-C₁-C₁₀ alkyl;
R² is selected from benzyl substituted by one or more of halogen; cyano; C₁-C₁₀ alkyl; C₁-C₁₀ alkoxy; aroyl; halo-C₁-C₁₀ alkyl; aryl-C₁-C₁₀ alkoxy and C₁-C₁₀ alkoxycarbonyl; 2-naphthylmethyl; 1-(4-chlorophenyl)-5-(trifluoromethyl)-1H-pyrazol-4-ylmethyl; 2-(4-chlorophenyl)ethyl; 2,1,3-benzothiadiazol-5-ylmethyl; and 1-[5-(trifluoromethyl)]-1,3-benzothiazol-2-ylmethyl;
R³ is selected from C₁-C₁₀ alkyl and aryl substituted by one or more of halogen;
R⁴ is selected from ethyl; isobutyl; propyl; 3,3-dimethylbutyl; C₁-C₁₀ alkyl substituted by one or more of hydroxy, oxo, C₁-C₁₀ alkoxy, C₁-C₁₀ alkoxycarbonylamino, tri-C₁-C₁₀ alkylsilyl, tri-C₁-C₁₀ alkylsilyloxy, C₁-C₁₀ alkylsulfonyl and aryloxy, wherein the aryloxy is substituted by one or more of halo-C₁-C₁₀ alkyl; amino-C₁-C₁₀ alkyl substituted by oxo; di-C₁-C₁₀ alkylamino-C₁-C₁₀ alkyl unsubstituted or substituted by one or more of oxo; halo-C₁-C₁₀ alkyl unsubstituted or substituted by one or more of hydroxy; C₁-C₁₀ alkoxycarbonyl-C₁-C₁₀ alkyl; C₂-C₁₀ alkenyl; C₃-C₁₀ cycloalkyl-C₁-C₁₀ alkyl unsubstituted or substituted by oxo; aryl-C₁-C₁₀ alkyl unsubstituted or substituted by one or more of halogen, C₁-C₁₀ alkoxy, halo-C₁-C₁₀ alkyl, halo-C₁-C₁₀ alkoxy, halo-C₁-C₁₀ alkylthio, C₁-C₁₀ alkylsulfonyl, oxo and heteroaryl; heteroaryl-C₁-C₁₀ alkyl unsubstituted or substituted by one or more of halogen, C₁-C₁₀ alkyl, C₁-C₁₀ alkylsulfonyl, halo-C₁-C₁₀ alkyl, oxo and aryl, wherein the aryl group is unsubstituted or substituted by halogen; heterocyclyl-C₁-C₁₀ alkyl unsubstituted or substituted by one or more of halogen, oxo and aryl;
with the proviso that the compound is not:
1-benzyl-3-isobutylxanthine;
1-benzyl-3-butylxanthine;
1-(4-chlorobenzyl)-3-ethyl-8-isopropylxanthine;
1,3-dibenzylxanthine; and
1,3-di-(4-chlorobenzyl)-8-isopropylxanthine.

In another embodiment, the present invention relates the compound above, wherein R² is selected from benzyl substituted by one or more of halogen; cyano; C₁-C₁₀ alkyl; C₁-C₁₀ alkoxy; aroyl; halo-C₁-C₁₀ alkyl; aryl-C₁-C₁₀ alkoxy and C₁-C₁₀ alkoxycarbonyl.

In another embodiment, the present invention relates the compound above, wherein R¹ is selected from bromo; methyl; ethyl; tert-butyl; methoxy; 1-hydroxyethyl; methoxymethyl; cyclobutyl; cyclopentyl; cyclohexyl; amino substituted by one or more of methyl, etyl and 2-methoxyethyl; azetidin-1-yl; morpholin-4-yl; piperazin-1-yl substituted by one or more of methyl; piperidin-1-yl unsubstituted or substituted by one or more of methoxy; pyrrolidin-1-yl unsubstituted or substituted by one or more of methoxymethyl, dimethylamino, oxo and pyrrolidin-1-ylmethyl; tetrahydrofuran-3-yl; and thiomorpholin-4-yl.

In another embodiment, the present invention relates the compound above, wherein R² is selected from benzyl substituted by one or more of bromo, chloro, fluoro, cyano, isopropyl, methoxy, benzoyl, trifluoromethyl, benzyloxy and carbomethoxy; 2-naphthylmethyl; 1-(4-chlorophenyl)-5-(trifluoromethyl)-1H-pyrazol-4-ylmethyl; 2-(4-chlorophenyl)ethyl; 2,1,3-benzothiadiazol-5-ylmethyl; and 1-[5-(trifluoromethyl)]-1,3-benzothiazol-2-ylmethyl.

In another embodiment, the present invention relates the compound above, wherein R² is selected from benzyl substituted by one or more of bromo, chloro, fluoro, cyano, isopropyl, methoxy, benzoyl, trifluoromethyl, benzyloxy and carbomethoxy.

In another embodiment, the present invention relates the compound above, wherein R³ is selected from methyl; ethyl; isopropyl; and 4-fluorophenyl.

In another embodiment, the present invention relates the compound above, wherein R⁴ is selected from ethyl; isobutyl; propyl; 3,3-dimethylbutyl; 3-hydroxypropyl; 2,3-dihydroxypropyl; 2-oxobutyl; 3,3-dimethyl-2-oxobutyl; 2-methoxyethyl; 2,2-dimethoxyethyl; 3-tert-butoxypropyl; 2-tert-butoxy-2-oxoethyl; 2-tert-butoxycarbonylaminoethyl; 2-(trimethylsilyl)ethyl; trimethylsilylmethyl; 2-tert-butyl(dimethyl)silyloxyethyl; 3-(tert-butylsulfonyl)propyl; 3-[4-(trifluoromethyl)phenoxy]propyl; 2-amino-2-oxoethyl; 2-diethylaminoethyl; 2-diisopropylamino-2-oxoethyl; 3,3,3-trifluoropropyl; 4,4,4-trifluorobutyl; 3,3,3-trifluoro-2-hydroxypropyl; carbomethoxymethyl; allyl; cyclohexylmethyl; 4-cyclohexylbutyl; 2-[(3S,5S,7S)-adamantan-1-yl]-2-oxoethyl; benzyl unsubstituted or substituted by one or more of chloro, methoxy, trifluoromethyl, difluoromethoxy, trifluoromethylthio, methylsulfonyl and 1H-pyrazol-1-yl; 2-oxo-2-phenylethyl; 3-chloro-4-isopropylsulfonyl-2-thienylmethyl; 1-(4-chlorophenyl)-5-(trifluoromethyl)-1H-pyrazol-4-ylmethyl; 3-(1H-imidazol-1-yl)propyl; 5-methylisoxazol-3-ylmethyl; 5-methyl-3-phenylisoxazol-4-ylmethyl; 2-oxo-2-pyridin-4-ylethyl; 2-(1H-pyrrol-1-yl)ethyl; pyridin-2-ylmethyl; pyridin-3-ylmethyl; 2-(3,3-difluoropyrrolidin-1-yl)-2-oxoethyl; 2,3-dihydro-1,4-benzodioxin-2-ylmethyl; 3-(1,4-dioxa-8-azaspiro[4.5]dec-8-yl)propyl; 1,3-dioxolan-2-ylmethyl; (2R)-5-oxopyrrolidin-2-ylmethyl; (2S)-5-oxopyrrolidin-2-ylmethyl; 3-(4-phenylpiperazin-1-yl)propyl; and 3-pyrrolidin-1-ylpropyl.

In another embodiment, the present invention relates to the compounds as denoted in Examples 1, 2, 4, 6-16, 18-44, 46-52, 54, 56-64, 66-72, 74-89, and 91-96.

The compounds of the general formula (I) may be prepared by a process, wherein a compound of formula (II)

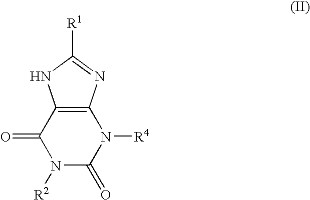

wherein R¹, R², and R⁴ are as defined above, is reacted with a compound of formula R³—X in the presence of a suitable base in a suitable solvent, wherein R³ is as defined above, and X is a leaving group.

One suitable base is potassium carbonate. One suitable solvent is DMF. Examples of leaving groups are halide groups, alkylsulfonate and arylsulfonate groups.

The general terms used in the definition of formula (I) have the following meanings:

C₁-C₁₀ alkyl is a straight or branched alkyl group, having from 1 to 10 carbon atoms, for example methyl, ethyl, n-propyl, isopropyl, n-butyl, isobutyl, secondary butyl, tertiary butyl, pentyl, isopentyl, hexyl or heptyl.

C₂-C₁₀ alkenyl is a straight or branched alkenyl group, having 2 to 10 carbon atoms, for example vinyl, allyl, isopropenyl and 1-butenyl.

C₃-C₁₀ cycloalkyl is a cyclic alkyl, having 3 to 10 carbon atoms such as cyclopropyl, cyclobutyl, cyclopentyl, cyclohexyl and adamantyl.

C₁-C₁₀ alkoxy is an alkoxy group having 1 to 10 carbon atoms, for example methoxy, ethoxy, n-propoxy, n-butoxy, isopropoxy, isobutoxy, secondary butoxy, tertiary butoxy, pentoxy, hexoxy or a heptoxy group.

The term aryl is herein defined as an aromatic ring having from 6 to 14 carbon atoms including both single rings and polycyclic compounds, such as phenyl, benzyl or naphthyl.

The term aroyl is herein defined as an aryl group bonded to a carbonyl group, such as benzoyl.

The term heteroaryl is herein defined as an aromatic ring having 3 to 14 carbon atoms, including both single rings and polycyclic compounds in which one or several of the ring atoms is either oxygen, nitrogen or sulphur, such as pyrazolyl, benzothiadiazolyl, benzothiazolyl, thienyl, imidazolyl, isoxazolyl, pyridinyl and pyrrolyl.

The term heterocyclyl is herein defined as a saturated or unsaturated non-aromatic ring having 3 to 14 carbon atoms, including both single rings and polycyclic compounds in which one or several of the ring atoms is either oxygen, nitrogen or sulphur, such as azetidinyl, morpholinyl, piperazinyl, piperidinyl, pyrrolidinyl, tetrahydrofuranyl, thiomorpholinyl, 2,3-dihydro-1,4-benzodioxinyl, 1,4-dioxa-8-azaspiro[4.5]dec-8-yl and 1,3-dioxolanyl.

Halogen as used herein is selected from chlorine, fluorine, bromine or iodine.

When two or more groups are used in connection with each other, it means that each group is substituted by the immediately preceding group. For instance, C₁-C₁₀ alkoxy-C₁-C₁₀ alkyl means a C₁-C₁₀ alkyl group substituted by a C₁-C₁₀ alkoxy group.

When a group is substituted by two or more further groups, these further groups need not be the same. For instance, in di-C₁-C₁₀ alkylamino, the both C₁-C₁₀ alkyl groups may be the same or different C₁-C₁₀ alkyl groups.

When the compounds of formula (I) have at least one asymmetric carbon atom, they can exist in several stereochemical forms. The present invention includes the mixture of isomers as well as the individual stereoisomers. The present invention further includes geometrical isomers, rotational isomers, enantiomers, racemates and diastereomers.

Where applicable, the compounds of formula (I) may be used in neutral form, e.g. as a carboxylic acid, or in the form of a salt, preferably a pharmaceutically acceptable salt such as the sodium, potassium, ammonium, calcium or magnesium salt of the compound at issue.

The compounds of formula (I) are useful as positive allosteric GBR (GABA_{B} receptor) modulators. A positive allosteric modulator of the GABA_{B} receptor is defined as a compound which makes the GABA_{B} receptor more sensitive to GABA and GABA_{B} receptor agonists by binding to the GABA_{B} receptor protein at a site different from that used by the endogenous ligand. The positive allosteric GBR modulator acts synergistically with an agonist and increases potency and/or intrinsic efficacy of the GABA_{B} receptor agonist. It has also been shown that positive allosteric modulators acting at the GABA_{B} receptor can produce an agonistic effect. Therefore, compounds of formula (I) can be effective as full or partial agonists.

The compounds may be used as a positive allosteric GABA_{B} receptor modulator. Also envisaged is a pharmaceutical composition comprising a compound above as an active ingredient and a pharmaceutically acceptable carrier or diluent.

A further aspect of the invention is a compound of the formula (I) above including the compounds as excluded in the proviso of claim 1 for use in therapy.

As a consequence of the GABA_{B} receptor becoming more sensitive to GABA_{B} receptor agonists upon the administration of a positive allosteric modulator, an increased inhibition of transient lower esophageal sphincter relaxations (TLESR) for a GABA_{B} agonist is observed. Consequently, the present invention is directed to the use of a positive allosteric GABA_{B} receptor modulator according to formula (I), optionally in combination with a GABA_{B} receptor agonist, for the preparation of a medicament for the inhibition of transient lower esophageal sphincter relaxations (TLESRs).

In another embodiment, the invention relates to a compound of formula (I), optionally in combination with a GABA_{B} receptor agonist, for use in the inhibition of transient lower esophageal sphincter relaxations (TLESR).

A further aspect of the invention is the use of a compound of formula (I), optionally in combination with a GABA_{B} receptor agonist, for the manufacture of a medicament for the prevention of reflux.

In another embodiment, the invention relates to a compound of formula (I), optionally in combination with a GABA_{B} receptor agonist, for use in the prevention of reflux.

Still a further aspect of the invention is the use of a compound of formula (I), optionally in combination with a GABA_{B} receptor agonist, for the manufacture of a medicament for the treatment of gastroesophageal reflux disease (GERD).

In another embodiment, the invention relates to a compound of formula (I), optionally in combination with a GABA_{B} receptor agonist, for use in the treatment of gastroesophageal reflux disease (GERD).

Effective management of regurgitation in infants would be an important way of preventing, as well as curing lung disease due to aspiration of regurgitated gastric contents, and for managing failure to thrive, inter alia due to excessive loss of ingested nutrient. Thus, a further aspect of the invention is the use of a compound of formula (I), optionally in combination with a GABA_{B} receptor agonist, for the manufacture of a medicament for the treatment of lung disease.

Another aspect of the invention is the use of a compound of formula (I), optionally in combination with a GABA_{B} receptor agonist, for the manufacture of a medicament for the management of failure to thrive.

Another aspect of the invention is the use of a compound of formula (I), optionally in combination with a GABA_{B} receptor agonist, for the manufacture of a medicament for the treatment or prevention of asthma, such as reflux-related asthma.

A further aspect of the invention is the use of a compound of formula (I), optionally in combination with a GABA_{B} receptor agonist, for the manufacture of a medicament for the treatment or prevention of laryngitis or chronic laryngitis.

A further aspect of the present invention is a method for the inhibition of transient lower esophageal sphincter relaxations (TLESRs), whereby a pharmaceutically and pharmacologically effective amount of a compound of formula (I), optionally in combination with a GABA_{B} receptor agonist, is administered to subject in need of such inhibition.

Another aspect of the invention is a method for the prevention of reflux, whereby a pharmaceutically and pharmacologically effective amount of a compound of formula (I), optionally in combination with a GABA_{B} receptor agonist, is administered to a subject in need of such prevention.

Still a further aspect of the invention is a method for the treatment of gastroesophageal reflux disease (GERD), whereby a pharmaceutically and pharmacologically effective amount of a compound of formula (I), optionally in combination with a GABA_{B} receptor agonist, is administered to a subject in need of such treatment.

Another aspect of the present invention is a method for the treatment or prevention of regurgitation, whereby a pharmaceutically and pharmacologically effective amount of a compound of formula (I), optionally in combination with a GABA_{B} receptor agonist, is administered to a subject in need of such treatment.

Yet another aspect of the invention is a method for the treatment or prevention of regurgitation in infants, whereby a pharmaceutically and pharmacologically effective amount of a compound of formula (I), optionally in combination with a GABA_{B} receptor agonist, is administered to a subject in need of such treatment.

Still a further aspect of the invention is a method for the treatment, prevention or inhibition of lung disease, whereby a pharmaceutically and pharmacologically effective amount of a compound of formula (I), optionally in combination with a GABA_{B} receptor agonist, is administered to a subject in need of such treatment. The lung disease to be treated may inter alia be due to aspiration of regurgitated gastric contents.

Still a further aspect of the invention is a method for the management of failure to thrive, whereby a pharmaceutically and pharmacologically effective amount of a compound of formula (I), optionally in combination with a GABA_{B} receptor agonist, is administered to a subject in need of such treatment.

A further aspect of the invention is a method for the treatment or prevention of asthma, such as reflux-related asthma, whereby a pharmaceutically and pharmacologically effective amount of a compound of formula (I), optionally in combination with a GABA_{B} receptor agonist, is administered to a subject in need of such treatment.

A further aspect of the invention is a method for the treatment or prevention of laryngitis or chronic laryngitis, whereby a pharmaceutically and pharmacologically effective amount of a compound of formula (I), optionally in combination with a GABA_{B} receptor agonist, is administered to a subject in need of such treatment.

A further embodiment is the use of a compound of formula (I), optionally in combination with a GABA_{B} receptor agonist, for the manufacture of a medicament for the treatment of a functional gastrointestinal disorder (FGD). Another aspect of the invention is a method for the treatment of a functional gastrointestinal disorder, whereby an effective amount of a compound of formula (I), optionally in combination with a GABA_{B} receptor agonist, is administered to a subject suffering from said condition.

In another embodiment, the invention relates to a compound of formula (I), optionally in combination with a GABA_{B} receptor agonist, for use in the treatment of a functional gastrointestinal disorder.

A further embodiment is the use of a compound of formula (I), optionally in combination with a GABA_{B} receptor agonist, for the manufacture of a medicament for the treatment of functional dyspepsia. Another aspect of the invention is a method for the treatment of functional dyspepsia, whereby an effective amount of a compound of formula (I), optionally in combination with a GABA_{B} receptor agonist, is administered to a subject suffering from said condition.

In another embodiment, the invention relates to a compound of formula (I), optionally in combination with a GABA_{B} receptor agonist, for use in the treatment of functional dyspepsia.

Functional dyspepsia refers to pain or discomfort centered in the upper abdomen. Discomfort may be characterized by or combined with upper abdominal fullness, early satiety, bloating or nausea. Etiologically, patients with functional dyspepsia can be divided into two groups:
1—Those with an identifiable pathophysiological or microbiologic abnormality of uncertain clinical relevance (e.g. Helicobacter pylori gastritis, histological duodenitis, gallstones, visceral hypersensitivity, gastroduodenal dysmotility)
2—Patients with no identifiable explanation for the symptoms.

Functional dyspepsia can be diagnosed according to the following:

At least 12 weeks, which need not be consecutive within the preceding 12 months of
1—Persistent or recurrent dyspepsia (pain or discomfort centered in the upper abdomen) and
2—No evidence of organic disease (including at upper endoscopy) that is likely to explain the symptoms and
3—No evidence that dyspepsia is exclusively relieved by defecation or associated with the onset of a change in stool frequency or form.

Functional dyspepsia can be divided into subsets based on distinctive symptom patterns, such as ulcer-like dyspepsia, dysmotility-like dyspepsia and unspecified (non-specific) dyspepsia.

Currently existing therapy of functional dyspepsia is largely empirical and directed towards relief of prominent symptoms. The most commonly used therapies still include antidepressants.

A further aspect of the invention is the use of a compound according to formula (I), optionally in combination with a GABA_{B} receptor agonist, for the manufacture of a medicament for the treatment or prevention of irritable bowel syndrome (IBS), such as constipation predominant IBS, diarrhea predominant IBS or alternating bowel movement predominant IBS.

In another embodiment, the invention relates to a compound of formula (I), optionally in combination with a GABA_{B} receptor agonist, for use in the treatment or prevention of irritable bowel syndrome (IBS), such as constipation predominant IBS, diarrhea predominant IBS or alternating bowel movement predominant IBS.

A further aspect of the invention is a method for the treatment or prevention of irritable bowel syndrome (IBS), whereby a pharmaceutically and pharmacologically effective amount of a compound of formula (I), optionally in combination with a GABA_{B} receptor agonist, is administered to a subject in need of such treatment.

IBS is herein defined as a chronic functional disorder with specific symptoms that include continuous or recurrent abdominal pain and discomfort accompanied by altered bowel function, often with abdominal bloating and abdominal distension. It is generally divided into 3 subgroups according to the predominant bowel pattern:
1—diarrhea predominant
2—constipation predominant
3—alternating bowel movements.

Abdominal pain or discomfort is the hallmark of IBS and is present in the three subgroups. IBS symptoms have been categorized according to the Rome criteria and subsequently modified to the Rome II criteria. This conformity in describing the symptoms of IBS has helped to achieve consensus in designing and evaluating IBS clinical studies.

The Rome II diagnostic criteria are:
1—Presence of abdominal pain or discomfort for at least 12 weeks (not necessarily consecutively) out of the preceding year
2—Two or more of the following symptoms:
   a) Relief with defecation
   b) Onset associated with change in stool frequency
   c) Onset associated with change in stool consistency

A further aspect of the invention is the use of a compound according to formula (I), optionally in combination with a GABA_{B} receptor agonist, for the manufacture of a medicament for the treatment or prevention CNS disorders, such as anxiety.

A further aspect of the invention is a method for the treatment or prevention of CNS disorders, such as anxiety, whereby a pharmaceutically and pharmacologically effective amount of a compound of formula (I), optionally in combination with a GABA_{B} receptor agonist, is administered to a subject in need of such treatment.

A further aspect of the invention is the use of a compound according to formula (I), optionally in combination with a GABA_{B} receptor agonist, for the manufacture of a medicament for the treatment or prevention of depression.

A further aspect of the invention is a method for the treatment or prevention of depression, whereby a pharmaceutically and pharmacologically effective amount of a compound of formula (I), optionally in combination with a GABA_{B} receptor agonist, is administered to a subject in need of such treatment.

For the purpose of this invention, the term “agonist” should be understood as including full agonists as well as partial agonists, whereby a “partial agonist” should be understood as a compound capable of partially, but not fully, activating GABA_{B} receptors.

The wording “TLESR”, transient lower esophageal sphincter relaxations, is herein defined in accordance with Mittal, R. K., Holloway, R. H., Penagini, R., Blackshaw, L. A., Dent, J., 1995; Transient lower esophageal sphincter relaxation. Gastroenterology 109, pp. 601-610.

The wording “reflux” is defined as a condition when fluid from the stomach is being able to pass into the esophagus, since the mechanical barrier (the esophageal sphincter) is temporarily not functioning as desired at such times.

The wording “GERD”, gastroesophageal reflux disease, is defined in accordance with van Heerwarden, M. A., Smout A. J. P. M., 2000; Diagnosis of reflux disease. Baillière's Clin. Gastroenterol. 14, pp. 759-774.

A “combination” according to the invention may be present as a “fix combination” or as a “kit of parts combination”.

A “fix combination” is defined as a combination wherein (i) a compound of formula (I); and (ii) a GABA_{B} receptor agonist are present in one unit. One example of a “fix combination” is a pharmaceutical composition wherein (i) a compound of formula (I) and (ii) a GABA_{B} receptor agonist are present in admixture. Another example of a “fix combination” is a pharmaceutical composition wherein (i) a compound of formula (I) and (ii) a GABA_{B} receptor agonist; are present in one unit without being in admixture.

A “kit of parts combination” is defined as a combination wherein (i) a compound of formula (I) and (ii) a GABA_{B} receptor agonist are present in more than one unit. One example of a “kit of parts combination” is a combination wherein (i) a compound of formula (I) and (ii) a GABA_{B} receptor agonist are present separately. The components of the “kit of parts combination” may be administered simultaneously, sequentially or separately, i.e. separately or together.

The term “positive allosteric modulator” is defined as a compound which makes a receptor more sensitive to receptor agonists by binding to the receptor protein at a site different from that used by the endogenous ligand.

The term “therapy” and the term “treatment” also include “prophylaxis” and/or prevention unless stated otherwise. The terms “therapeutic” and “therapeutically” should be construed accordingly.

### Pharmaceutical Formulations

The compound of formula (I) can be formulated alone or in combination with a GABA_{B} receptor agonist.

For clinical use, the compound of formula (I), optionally in combination with a GABA_{B} receptor agonist, is in accordance with the present invention suitably formulated into pharmaceutical formulations for oral administration. Also rectal, parenteral or any other route of administration may be contemplated to the skilled man in the art of formulations. Thus, the compound of formula (I), optionally in combination with a GABA_{B} receptor agonist, is formulated with a pharmaceutically and pharmacologically acceptable carrier or adjuvant. The carrier may be in the form of a solid, semi-solid or liquid diluent.

In the preparation of oral pharmaceutical formulations in accordance with the invention, the compound of formula (I), optionally in combination with a GABA_{B} receptor agonist, to be formulated is mixed with solid, powdered ingredients such as lactose, saccharose, sorbitol, mannitol, starch, amylopectin, cellulose derivatives, gelatin, or another suitable ingredient, as well as with disintegrating agents and lubricating agents such as magnesium stearate, calcium stearate, sodium stearyl fumarate and polyethylene glycol waxes. The mixture is then processed into granules or compressed into tablets.

Soft gelatine capsules may be prepared with capsules containing a mixture of a compound of formula (I), optionally in combination with a GABA_{B} receptor agonist, with vegetable oil, fat, or other suitable vehicle for soft gelatine capsules. Hard gelatine capsules may contain a compound of formula (I), optionally in combination with a GABA_{B} receptor agonist, in combination with solid powdered ingredients such as lactose, saccharose, sorbitol, mannitol, potato starch, corn starch, amylopectin, cellulose derivatives or gelatine.

Dosage units for rectal administration may be prepared (i) in the form of suppositories which contain the active substance(s) mixed with a neutral fat base; (ii) in the form of a gelatine rectal capsule which contains a compound of formula (I), optionally in combination with a GABA_{B} receptor agonist, in a mixture with a vegetable oil, paraffin oil, or other suitable vehicle for gelatine rectal capsules; (iii) in the form of a ready-made micro enema; or (iv) in the form of a dry micro enema formulation to be reconstituted in a suitable solvent just prior to administration.

Liquid preparations for oral administration may be prepared in the form of syrups or suspensions, e.g. solutions or suspensions, containing a compound of formula (I), optionally in combination with a GABA_{B} receptor agonist, and the remainder of the formulation consisting of sugar or sugar alcohols, and a mixture of ethanol, water, glycerol, propylene glycol and polyethylene glycol. If desired, such liquid preparations may contain colouring agents, flavouring agents, saccharine and carboxymethyl cellulose or other thickening agents. Liquid preparations for oral administration may also be prepared in the form of a dry powder to be reconstituted with a suitable solvent prior to use.

Solutions for parenteral administration may be prepared as a solution of a compound of formula (I), optionally in combination with a GABA_{B} receptor agonist, in a pharmaceutically acceptable solvent. These solutions may also contain stabilizing ingredients and/or buffering ingredients and are dispensed into unit doses in the form of ampoules or vials. Solutions for parenteral administration may also be prepared as a dry preparation to be reconstituted with a suitable solvent extemporaneously before use.

In one aspect of the present invention, a compound of formula (I), optionally in combination with a GABA_{B} receptor agonist, may be administered once or twice daily, depending on the severity of the patient's condition. A typical daily dose of the compounds of formula (I) is from 0.1 to 100 mg per kg body weight of the subject to be treated, but this will depend on various factors such as the route of administration, the age and weight of the patient as well as of the severity of the patient's condition.

### Methods of Preparation

Hereinbelow, Schemes 1-5 denote methods for preparation of the compounds according to the present invention.

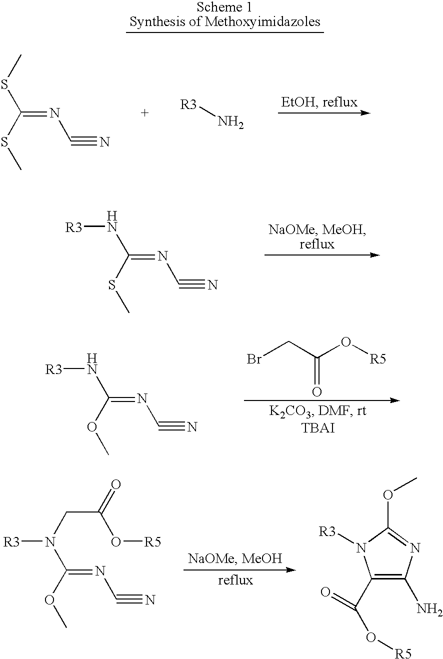

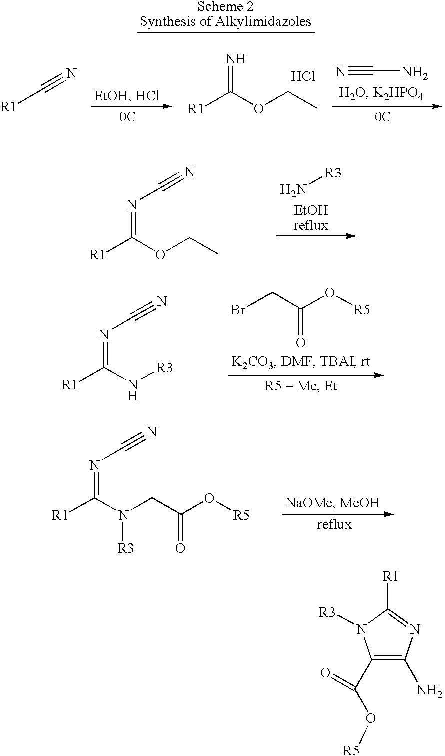

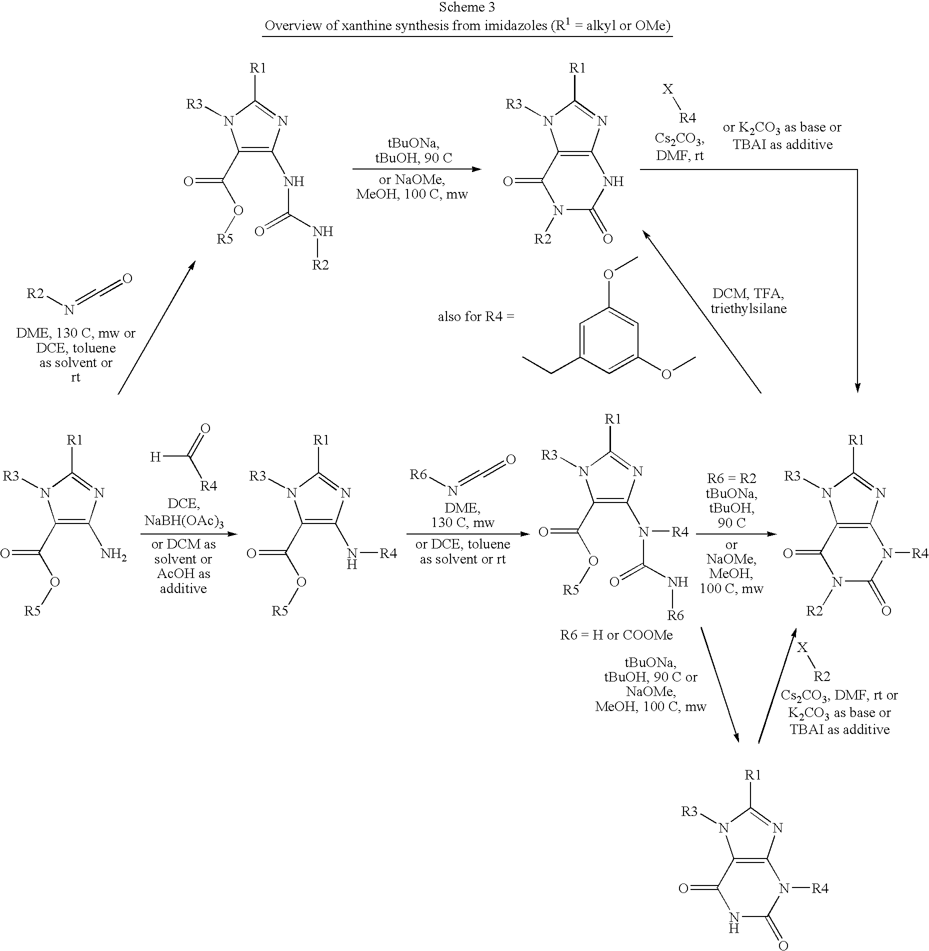

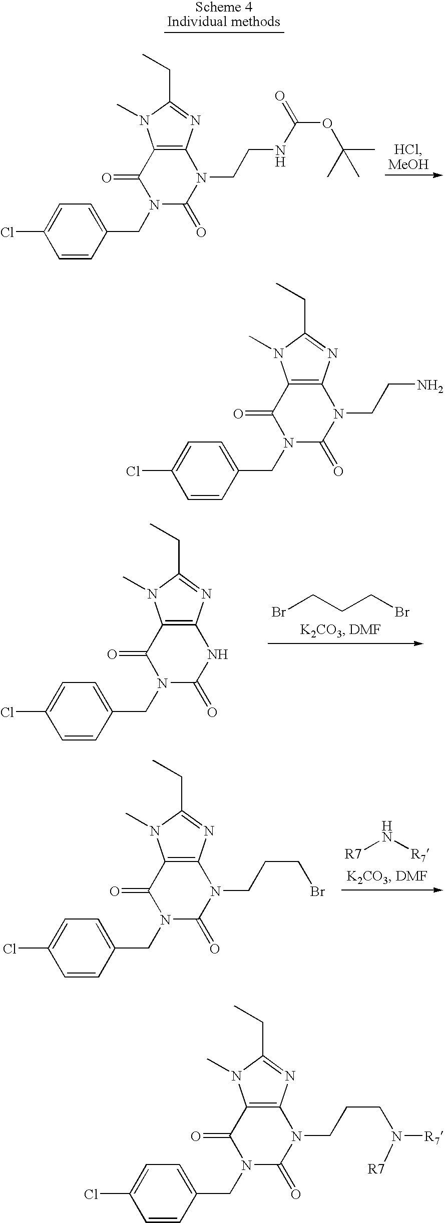

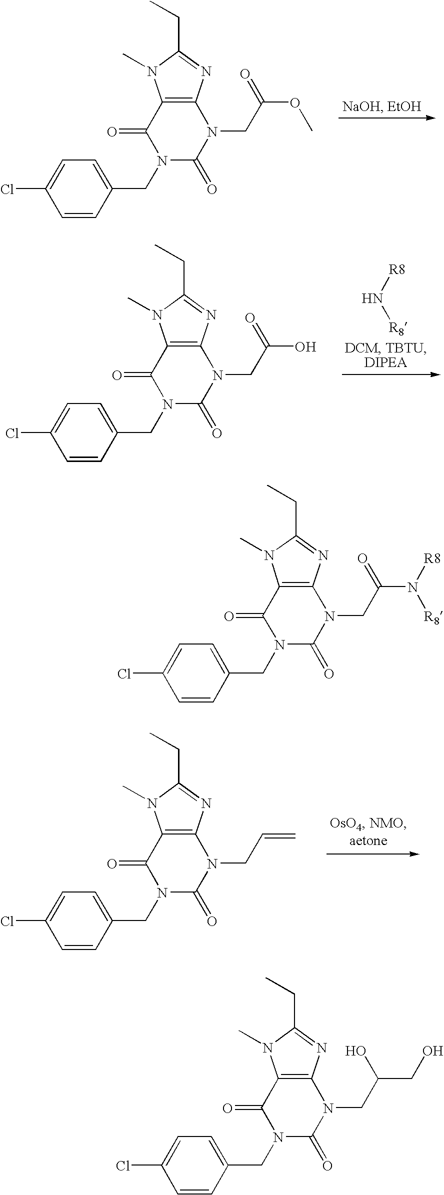

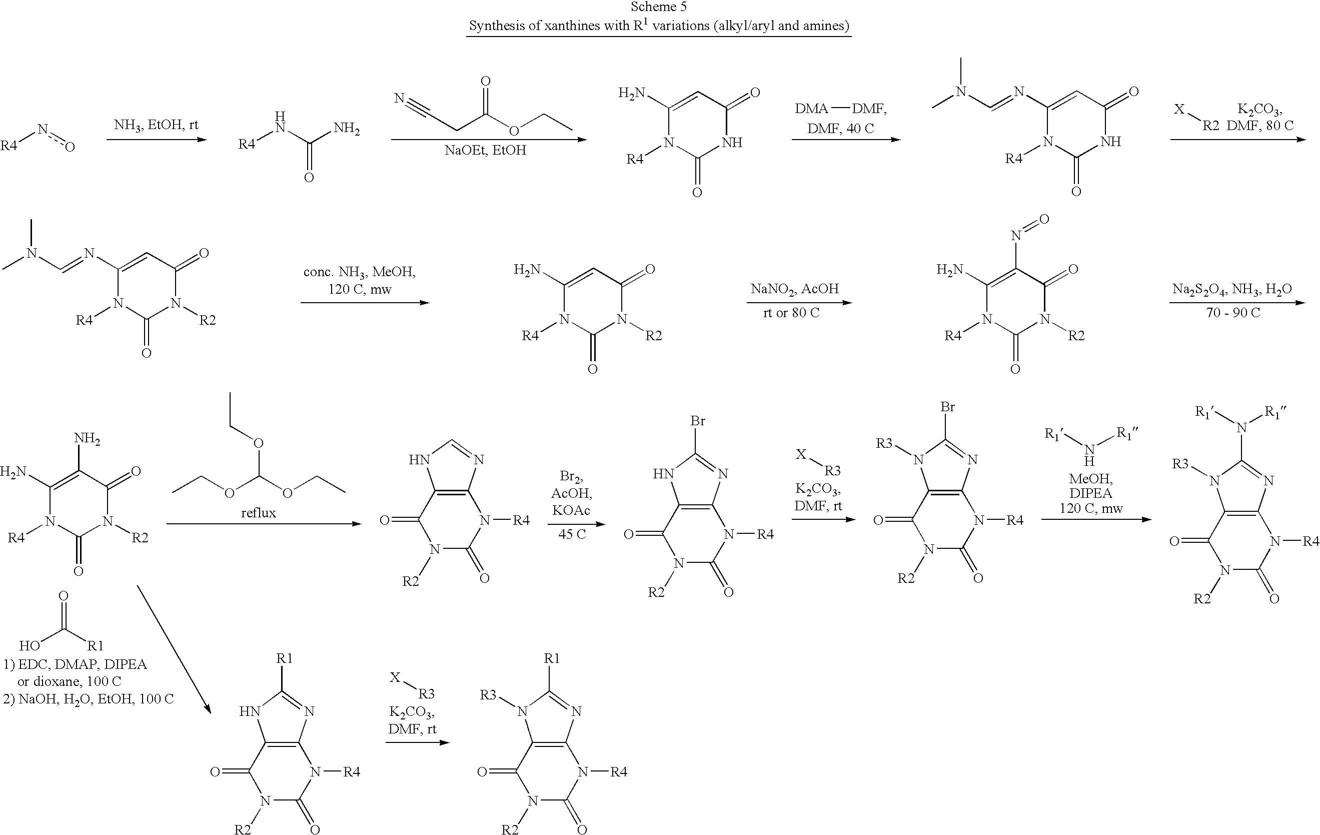

### Abbreviations

DCM dichloromethane

### DIPEA N,N-diisopropylethylamine

DMF N,N′-dimethylformamide

### DMAP N,N-dimethylaminopyridine

DMSO dimethylsulfoxide
EDC 1-ethyl-3-(3-dimethylaminopropyl)carbodiimide hydrochloride
EtOAc ethyl acetate
EtOH ethanol
HPFC high performance flash chromatography
HPLC high performance liquid chromatography
LC-MS liquid chromatography mass spectroscopy
MeCN acetonitrile
MeOH methanol
NaOMe sodium methoxide
NMR nuclear magnetic resonance
TBTU O-(benzotriazol-1-yl)-N,N,N′,N′-tetramethyluronium tetrafluoroborate
TEA triethylamine
Tert tertiary
TFA trifluoroacetic acid
THF tetrahydrofuran
UV ultra violet
atm atmosphere
rt room temperature
h hour(s)
min minutes
br broad
s singlet
d doublet
t triplet
q quartet
m multiplet
sep septet
dd double doublet
td triple doublet

### General Experimental Procedures

Phase Separator from IST was used. Flash column chromatography employed normal phase silica gel 60 (0.040-0.063 mm, Merck) or IST Isolute®SPE columns normal phase silica gel or Biotage Horizon™ HPFC System using silica FLASH+™ HPFC™ Cartridges. HPLC purifications were performed on either a Gilson preparative HPLC system with gradient pump system 333/334, GX-281 injector, UV/VIS detector 155. Trilution LC v. 1.4 software. In acidic system equipped with an Kromasil C8 10 μm 250×20 ID mm column or Kromasil C8 10 μm 250×50 ID mm column and as gradient: mobile phase (buffer): H₂O/MeCN/FA 95/5/0.2 and mobile phase (organic): MeCN. In neutral system equipped with an Kromasil C8 10 μm 250×20 ID mm column or Kromasil C8 10 μm 250×50 ID mm column and as gradient: mobile phase (buffer): MeCN/0.1M NH₄OAc 5/95 and mobile phase (organic): MeCN. In basic system equipped with an XBridge C18 10 μm 250×19 ID mm column or XBridge C18 10 μm 250×50 ID mm column and as gradient: mobile phase (buffer): H₂O/MeCN/NH₃ 95/5/0.2 and mobile phase (organic): MeCN. Or on a Waters preparative HPLC system equipped with a Kromasil C8 10 mm 250 mm×21.2 mm column and a gradient mobile phase (buffer): MeCN/0.1M NH₄OAc 5/95 and mobile phase (organic): MeCN or on a Waters FractionLynx HPLC system with a mass triggered fraction collector, equipped with a Xbridge Prep C18 5μ 19 mm×150 mm column using MeCN/NH₃ buffer system with a gradient from 95% mobilphase A (0.2% NH₃ in water, pH 10) to 95% mobilphase B (100% MeCN) unless otherwise stated. ¹H NMR and ¹³C NMR measurements were performed on a BRUKER ACP 300 or on a Varian Inova 400, 500 or 600 spectrometer, operating at ¹H frequencies of 300, 400, 500, 600 MHz, respectively, and ¹³C frequencies of 75, 100, 125 and 150 MHz, respectively. Chemical shifts are given in 6 values (ppm) with the solvents used as internal standard, unless otherwise stated. Microwave heating was performed using single node heating in a Smith Creator or Emrys Optimizer from Personal Chemistry, Uppsala, Sweden. Mass spectral data were obtained using a Micromass LCT or Waters Q-T of micro system and, where appropriate, either positive ion data or negative ion data were collected.

Compound names generated by ACD/Name Release 9.0. Product Version: 9.04 (Build 6210, 20 Jul. 2005).

### Explanation to Plate-NMR:

*The solutions are taken from a concentrated sample dissolved in (CH₃)₂SO and are diluted with (CD₃)₂SO. Since a substantial amount of (CH₃)₂SO is present in the sample, first a pre-scan is run and analysed to automatically suppress the (CH₃)₂SO (2.54 ppm) and H₂O (3.3 ppm) peaks. This means that in this so-called wet 1D experiment the intensity of peaks that reside in these areas around 3.3 ppm and 2.54 ppm are reduced. Furthermore impurities are seen in the spectrum which give rise to a triplet at 1.12 ppm, a singlet at 2.96 ppm and two multiplets between 2.76-2.70 ppm and 2.61-2.55 ppm. Most probably these impurities are dimethylsulfone and diethylsulfoxide.

### Starting Material and Intermediates

### REFERENCE EXAMPLE 1

### Synthesis of 1-(4-chlorobenzyl)-8-ethyl-7-methyl-3,7-dihydro-1H-purine-2,6-dione

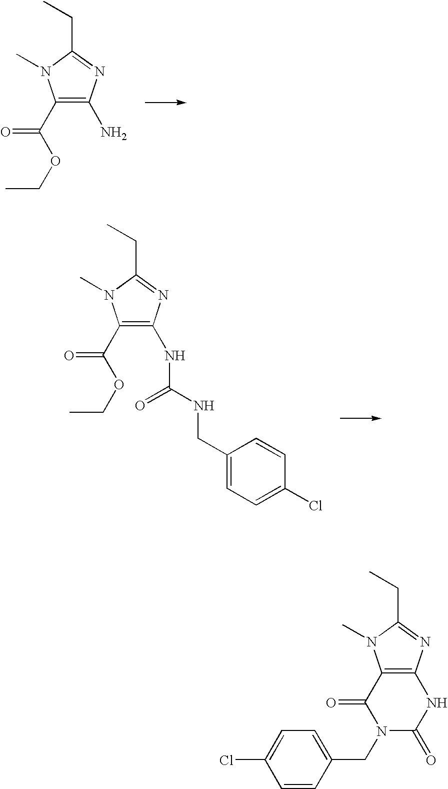

1.01 g (5.12 mmol) ethyl 4-amino-2-ethyl-1-methyl-1H-imidazole-5-carboxylate was dissolved in 11 mL toluene, then 0.762 mL 4-chlorobenzyl isocyanate was added. The resulting mixture was heated to 120° C. for 1 h in a sealed vial using microwave heating, then the solvents were evaporated. The residue was dissolved in 15 mL 0.5 M NaOMe in MeOH (7.5 mmol) and heated to 100° C. for 30 min in a sealed vial using microwave heating. 0.464 ml (8.11 mmol) acetic acid was added and the resulting precipitate collected, washed with water and dried. 1.5 g (4.71 mmol, 93%) of the pure title compound was isolated.

¹H-NMR (400 MHz, CDCl₃) δ 11.46 (br, 1H), 7.46-7.41 (m, 2H), 7.30-7.24 (m, 2H), 5.14 (s, 2H), 3.93 (s, 3H), 2.88 (q, 2H), 1.40 (t, 3H)

MS m/z 319.062 (M+H)⁺

### REFERENCE EXAMPLE 2

### Synthesis of ethyl 4-amino-2-ethyl-1-methyl-1H-imidazole-5-carboxylate

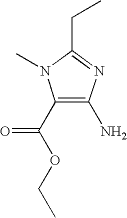

NaOMe (24.6 g, 0.4567 mol) in EtOH (IL) was added dropwise to a stirred solution of ethyl N—[N-cyanopropanimidoyl]-N-methylglycinate (60 g, 0.3045 mol) in EtOH (200 mL) under N₂ at rt and refluxed for 2 h. After completion of reaction, reaction mixture was cooled to rt and acetic acid (27.4 g, 0.4567 mol) was added to the reaction mixture and concentrated to afford crude product. The crude product was dissolved in EtOAc (600 mL), washed with saturated 10% NaHCO₃ solution, dried (Na₂SO₄) and concentrated under vacuum to afford crude pale yellow product. The pale yellow crude material was triturated with Et₂O to afford the title compound (23.8 g, 39.7%) as an off-white solid. R_{f}; 0.5 (CHCl₃: MeOH; 9:1).

¹H-NMR (500 MHz, CDCl₃) δ 4.77 (s, 2H), 4.26 (q, 7.1 Hz, 2H), 3.66 (s, 3H), 2.57 (q, 7.5 Hz, 2H), 1.33 (t, 7.1 Hz, 3H), 1.24 (t, 7.5 Hz, 3H).

MS m/z 186.10 (M+H)⁺.

### REFERENCE EXAMPLE 3

### Synthesis of ethyl N-[N-cyanopropanimidoyl]-N-methylglycinate

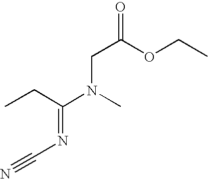

K₂CO₃ (261.4 g, 0.1.89 mol) was added to a stirred solution of N′-cyano-N-methylpropanimidamide (70 g, 0.6306 mol) in DMF (500 mL) under N₂ followed by tetrabutylammonium iodide (6.98 g, 0.019 mol). Then, ethylbromoacetate (157.9 g, 0.949 mol) was added to the reaction mixture at rt and continued stirring overnight. After completion of reaction, water (1 L) was added to the reaction mixture, extracted with ethyl acetate (3×600 mL), washed water (200 mL), brine (500 mL), dried (Na₂SO₄) and concentrated to afford the crude title compound (60 g, 48.3%) as a colorless solid. R_{f}; 0.6 (CHCl₃: MeOH; 9:1).

### REFERENCE EXAMPLE 4

### Synthesis of N′-cyano-N-methylpropanimidamide

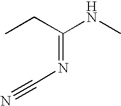

To a stirred solution of ethyl N-cyanopropanimidoate (80 g, 0.6349 mol) in EtOH (800 mL) was added 40% methylamine (19.72 g, 0.6349 mol) at rt and refluxed for 1 h. After completion of reaction, solvent was evaporated and partioned between water and ethyl acetate. Aqueous layer was extracted with EtOAc (3×100 mL), washed with brine (200 mL), dried (Na₂SO₄), and concentrated to afford the crude title compound (70 g, 99.4%) as a colorless solid.

### REFERENCE EXAMPLE 5

### Synthesis of ethyl N-cyanopropanimidoate

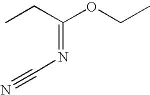

Cyanamide (45.86 g, 1.092 mol) was added to ethyl propanimidoate hydrochloride (120 g, 0.883 mol) in water (600 mL) followed by K₂HPO₄ (220 g, 1.264 mol) at 0° C.

The organic layer was separated and concentrated to afford the crude title compound (80 g, 71.9%) as a pale yellow liquid.

### REFERENCE EXAMPLE 6

### Synthesis of Ethyl Propanimidoate Hydrochloride

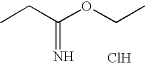

HCl gas was passed into propionitrile (60 g) in ethanol (550 mL) at 0° C. and the reaction mixture was kept for 19 h at 4° C. Then, the solvents were evaporated to afford the crude title compound (120 g, 85%) as a colorless solid.

The following compound was synthesized according to the reference examples 6-2:

### REFERENCE EXAMPLE 7

### Methyl 4-amino-2-ethyl-1-methyl-1H-imidazole-5-carboxylate

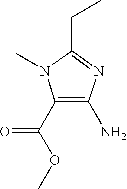

From 5.45 g propionitrile (0.099 mol), 3.4 g methyl 4-amino-2-ethyl-1-methyl-1H-imidazole-5-carboxylate (18.56 mmol, 18.7%) was isolated.

¹H-NMR (500 MHz, CDCl₃) δ 4.82 (br, 2H), 3.84 (s, 3H), 3.70 (s, 3H), 2.61 (q, 2H), 1.29 (t, 3H)

MS m/z 184.0 (M+H)⁺.

### REFERENCE EXAMPLE 8

### Methyl 4-amino-2-ethyl-1-(4-fluorophenyl)-1H-imidazole-5-carboxylate

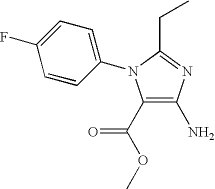

From 30 g propionitrile (0.545 mol), 20 g methyl 4-amino-2-ethyl-1-(4-fluorophenyl)-1H-imidazole-5-carboxylate (75.97 mmol, 13.9%) was isolated as a yellow solid.

¹H NMR (400 MHz, CD₃OD) δ 7.29-7.34 (m, 2H), 7.22-7.27 (m, 2H), 3.58 (s, 3H), 2.41 (q, 7.6 Hz, 2H), 1.13 (t, 7.6 Hz, 3H)

The following compound was synthesized according to reference example 1:

### REFERENCE EXAMPLE 9

### 1-(3,4-dichlorobenzyl)-8-ethyl-7-methyl-3,7-dihydro-1H-purine-2,6-dione

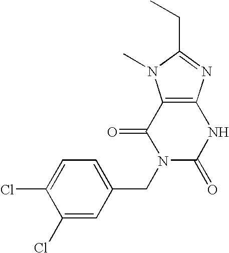

From 304 mg (1.54 mmol) ethyl 4-amino-2-ethyl-1-methyl-1H-imidazole-5-carboxylate, 479 mg (1.36 mmol, 88%) of the title compound was isolated.

¹H-NMR (400 MHz, CDCl₃) δ 10.69 (br, 1H), 7.55-7.52 (m, 1H), 7.39-7.27 (m, 2H), 5.08 (s, 2H), 3.83 (s, 3H), 2.81 (q, 2H), 1.36 (t, 3H)

MS m/z 352.96 (M+H)⁺

### REFERENCE EXAMPLE 10

### 1-(4-chlorobenzyl)-8-ethyl-7-(4-fluorophenyl)-3,7-dihydro-1H-purine-2,6-dione

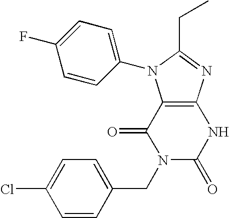

From 500 mg (1.90 mmol) methyl 4-amino-2-ethyl-1-(4-fluorophenyl)-1H-imidazole-5-carboxylate, 783 mg (1.96 mmol, 103%) of the crude title compound was isolated.

¹H NMR (400 MHz, CDCl₃) δ 11.70-11.50 (br, 1H), 7.37 (d, 2H), 7.34-7.27 (m, 2H), 7.25-7.16 (m, 4H), 5.05 (s, 2H), 2.71 (q, 2H), 1.27 (t, 3H).

MS m/z 399,401 (M+H)⁺.

### REFERENCE EXAMPLE 11

### Synthesis of 3-(3-bromopropyl)-1-(4-chlorobenzyl)-8-ethyl-7-methyl-3,7-dihydro-1H-purine-2,6-dione

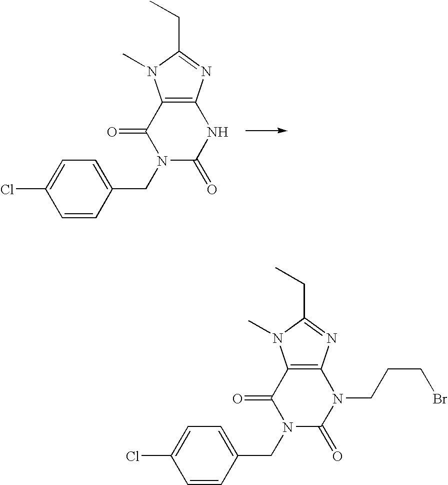

450 mg (1.41 mmol) 1-(4-Chlorobenzyl)-8-ethyl-7-methyl-3,7-dihydro-1H-purine-2,6-dione and Cs₂CO₃ (1.38 g, 4.24 mmol) were dissolved in DMF (10 mL) and 1,3-dibromopropane (2 mL, 14.1 mmol) dissolved in DMF (15 mL) was added dropwise. The reaction mixture was stirred at rt for 1.5 h. Water was added to the reaction mixture and extracted with EtOAc (3 times). The combined organic layers were washed with water twice. The organic layer was dried by filtration through a phase separator and evaporated to yield 470 mg (1.07 mmol, 76%) of the crude title compound.

¹H-NMR (400 MHz, CDCl₃) δ 7.46-7.33 (m, 2H), 7.29-7.13 (m, 2H), 5.11 (s, 2H), 4.19 (t, 2H), 3.87 (s, 3H), 2.71 (q, 2H), 2.38-2.26 (m, 2H), 1.20 (t, 3H)

MS m/z 439.1 (M+H)⁺

### REFERENCE EXAMPLE 12

### Synthesis of [1-(4-chlorobenzyl)-8-ethyl-7-methyl-2,6-dioxo-1,2,6,7-tetrahydro-3H-purin-3-yl]acetic acid

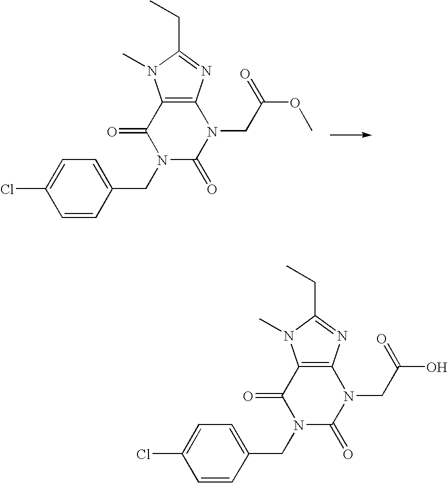

Methyl [1-(4-chlorobenzyl)-8-ethyl-7-methyl-2,6-dioxo-1,2,6,7-tetrahydro-3H-purin-3-yl]acetate (610 mg, 1.56 mmol) was dissolved in ethanol (9.6 mL), 5M NaOH (aq., 5 mL) was added, followed by additional ethanol and stirred at rt overnight. The solvents were evaporated. Diluted aq. HCl was added to the crude and extracted three times with dichloromethane. The combined organic layers were dried by filtration through a phase separator and evaporated to yield 400 mg (1.04 mmol, 67%) of the title compound

¹H-NMR (400 MHz, CDCl₃) δ 7.36-7.22 (m, 4H), 5.00 (s, 2H), 4.59 (s, 2H), 3.80 (s, 3H), 2.71 (q, 2H), 1.18 (t, 3H)

MS m/z 377,1010 (M+H)⁺

### REFERENCE EXAMPLE 13

### Synthesis of 8-ethyl-7-methyl-3-propyl-3,7-dihydro-1H-purine-2,6-dione

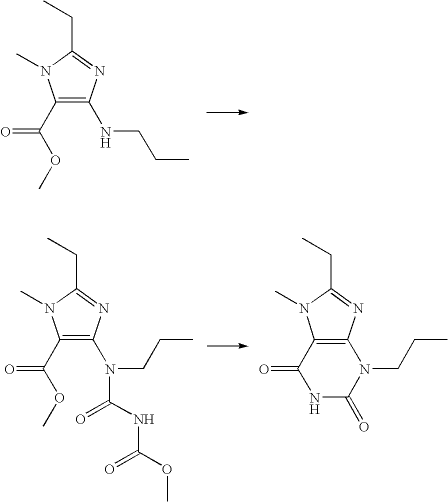

1.29 g (5.73 mmol) methyl 2-ethyl-1-methyl-4-(propylamino)-1H-imidazole-5-carboxylate was dissolved in 7 mL dimethoxyethane, then 772 mg (6.88 mmol) N-(methoxycarbonyl)isocyanate was added. The resulting mixture was stirred at rt for 30 min, then the solvents were evaporated, the residue dissolved in dichloromethane and washed with sat. NaHCO₃. The organic layer was dried by filtration through a phase separator and evaporated.

The residue was dissolved in 11 mL 0.5 M NaOMe in methanol and was heated to 70° C. for 2.5 h and 100° C. for 15 min in a sealed vial using microwave heating. After cooling to rt, 350 μl (6.13 mmol) of acetic acid was added, the formed precipitate collected, washed with water and dried. 917 mg (3.88 mmol, 68%) of the crude title compound was isolated.

¹H-NMR (500 MHz, CDCl₃) δ 8.21-8.12 (br, 1H) 4.03 (t, 2H), 3.90 (t, 3H), 2.77 (q, 2H), 1.86-1.76 (m, 2H), 1.36 (t, 3H), 0.98 (t, 3H)

MS m/z 237.09 (M+H)⁺

The following compounds were synthesized according to reference example 11:

### REFERENCE EXAMPLE 14

### 7,8-diethyl-3-propyl-3,7-dihydro-1H-purine-2,6-dione

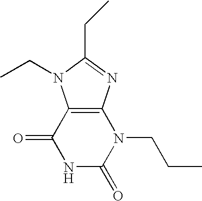

From 302 mg (1.26 mmol) Methyl 1,2-diethyl-4-(propylamino)-1H-imidazole-5-carboxylate, 196 mg (0.78 mmol, 62%) of the crude title compound was isolated.

MS m/z 251 (M+H)⁺.

### REFERENCE EXAMPLE 15

### 8-Ethyl-7-isopropyl-3-propyl-3,7-dihydro-1H-purine-2,6-dione

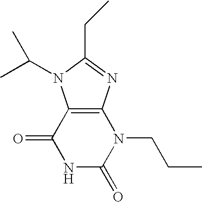

From 33 mg (0.13 mmol) methyl 2-ethyl-1-isopropyl-4-(propylamino)-1H-imidazole-5-carboxylate, 28 mg (0.106 mmol, 78%) of the crude title compound was isolated.

¹H NMR (400 MHz, CDCl₃) δ 4.70-5.55 (br, 1H), 3.98 (t, 2H), 2.77 (q, 2H), 1.81-1.70 (m, 2H), 1.59 (d, 6H), 1.29 (t, 3H), 0.93 (t, 3H).

MS m/z 265 (M+H)⁺.

### REFERENCE EXAMPLE 16

### 3-(2,4-dimethoxybenzyl)-8-ethyl-7-methyl-3,7-dihydro-1H-purine-2,6-dione

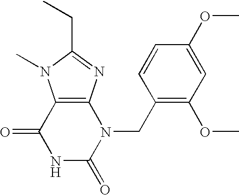

From 1.14 g (3.42 mmol) methyl 4-[(2,4-dimethoxybenzyl)amino]-2-ethyl-1-methyl-1H-imidazole-5-carboxylate, 880 mg (2.56 mmol, 75%) of the title compound was isolated.

¹H-NMR (500 MHz, CDCl₃) δ 7.86 (br, 1H), 6.97 (d, 8.3 Hz, 1H), 6.41-6.42 (m, 1H), 6.33-6.37 (m, 1H), 5.18 (s, 2H), 3.86 (s, 3H), 3.79 (s, 3H), 3.74 (s, 3H), 2.70 (q, 7.5 Hz, 2H), 1.29 (t, 7.5 Hz, 3H)

MS m/z 345 (M+H)⁺

### REFERENCE EXAMPLE 17

### Synthesis of methyl 2-ethyl-1-methyl-4-(propylamino)-1H-imidazole-5-carboxylate

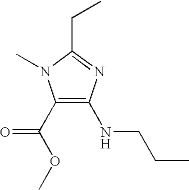

570 mg (2.96 mmol) methyl 4-amino-2-ethyl-1-methyl-1H-imidazole-5-carboxylate was dissolved in 7.5 mL DCM, then 232 μL (3.25 mmol) propionaldehyde and 950 mg (4.48 mmol) sodium triacetoxyborohydride was added. The resulting mixture was stirred at rt for 3 days, then more DCM was added and washed with sat. NaHCO₃. The organic layer was dried by filtration through a phase separator and evaporated. 670 mg (2.96 mmol, 100%) of the crude title compound was isolated.

MS m/z 226.16 (M+H)⁺

The following compounds were synthesized according to reference example 17:

### REFERENCE EXAMPLE 18

### Methyl 4-[(2,4-dimethoxybenzyl)amino]-2-ethyl-1-methyl-1H-imidazole-5-carboxylate

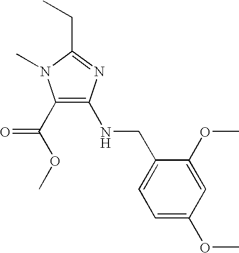

From 800 mg (4.37 mmol) methyl 4-amino-2-ethyl-1-methyl-1H-imidazole-5-carboxylate, 1.36 g (4.07 mmol, 93%) of the title compound was isolated.

¹H-NMR (400 MHz, CDCl₃) δ 7.24 (s, 1H), 6.37-6.44 (m, 2H), 5.83 (br, 1H), 4.51 (d, 6 Hz, 2H), 3.81 (s, 3H), 3.76 (s, 3H), 3.75 (s, 3H), 3.64 (s, 3H), 2.61 (q, 7.5 Hz, 2H), 1.24 (t, 7.5 Hz, 3H)

MS m/z 334 (M+H)⁺

### REFERENCE EXAMPLE 19

### Ethyl 2-ethyl-1-methyl-4-[(3,3,3-trifluoropropyl)amino]-1H-imidazole-5-carboxylate

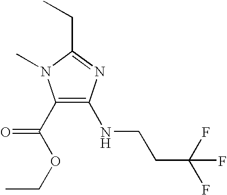

From 300 mg (1.52 mmol) ethyl 4-amino-2-ethyl-1-methyl-1H-imidazole-5-carboxylate using 1,2-dichloroethane as solvent, 449 mg (1.52 mmol, 100%) of the crude title compound was isolated.

¹H NMR (400 MHz, CDCl₃) δ 5.70-5.50 (br, 1H), 4.17 (q, 2H), 3.63-3.55 (m, 5H), 2.53 (q, 2H), 2.40-2.26 (m, 2H), 1.24 (t, 3H), 1.15 (t, 3H).

MS m/z 294 (M+H)⁺.

### REFERENCE EXAMPLE 20

### Ethyl 2-ethyl-1-methyl-4-[(4,4,4-trifluorobutyl)amino]-1H-imidazole-5-carboxylate

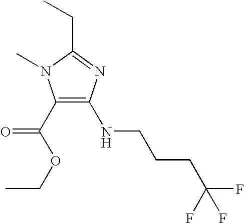

From 300 mg (1.52 mmol) ethyl 4-amino-2-ethyl-1-methyl-1H-imidazole-5-carboxylate using 1,2-dichloroethane as solvent, 518 mg (1.68 mmol, 110%) of the crude title compound was isolated.

¹H NMR (400 MHz, CDCl₃) δ 5.90-5.30 (br, 1H), 4.25 (q, 2H), 3.64 (s, 3H), 3.45 (t, 2H), 2.60 (q, 2H), 2.22-2.07 (m, 2H), 1.88-1.78 (m, 2H), 1.31 (t, 3H), 1.21 (t, 3H).

MS m/z 308 (M+H)⁺.

### REFERENCE EXAMPLE 21

### Methyl 1,2-diethyl-4-(propylamino)-1H-imidazole-5-carboxylate

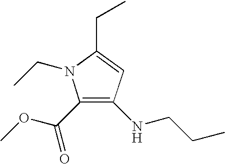

From 300 mg (1.52 mmol) methyl 4-amino-1,2-diethyl-1H-imidazole-5-carboxylate, 302 mg (1.26 mmol, 83%) of the title compound were isolated.

¹H NMR (400 MHz, CDCl₃) δ 6.30-5.30 (br, 1H), 4.02 (q, 2H), 3.71 (s, 3H), 3.27 (t, 2H), 2.54 (q, 2H), 1.60-1.45 (m, 2H), 1.22-1.13 (m, 6H), 0.86 (t, 3H).

MS m/z 240 (M+H)⁺.

### REFERENCE EXAMPLE 22

### Methyl 2-ethyl-1-isopropyl-4-(propylamino)-1H-imidazole-5-carboxylate

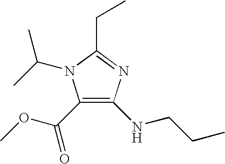

From 244 mg (0.46 mmol) methyl 4-amino-2-ethyl-1-isopropyl-1H-imidazole-5-carboxylate, 33 mg (0.13 mmol, 28%) of the title compound was isolated.

¹H NMR (400 MHz, CDCl₃) δ 5.70-5.56 (br, 1H), 4.90-4.70 (br, 1H), 3.77 (s, 3H), 3.37-3.27 (br, 2H), 2.66 (q, 2H), 1.65-1.53 (m, 2H), 1.46 (d, 6H), 1.23 (t, 3H), 0.93 (t, 3H).

MS m/z 254 (M+H)⁺.

The following compounds were synthesized according to the reference examples 2 and 3:

### REFERENCE EXAMPLE 23

### Methyl 4-amino-1,2-diethyl-1H-imidazole-5-carboxylate

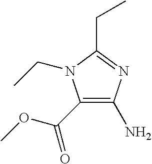

From 1.39 g (11.1 mmol, crude, ca. 70% pure) (1Z)-N′-cyano-N-ethylpropanimidamide, 686 mg (2.43 mmol, 30%, purity ca. 70%) of the title compound was isolated.

¹H NMR (400 MHz, CDCl₃) δ 4.90-4.70 (br, 2H), 4.11 (q, 2H), 3.79 (s, 3H), 2.56 (q, 2H), 1.29-1.22 (m, 6H).

MS m/z 198 (M+H)⁺.

### REFERENCE EXAMPLE 24

### Methyl 4-amino-2-ethyl-1-isopropyl-1H-imidazole-5-carboxylate

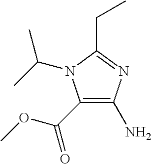

From 200 mg (1.44 mmol) (1Z)-N′-cyano-N-isopropylpropanimidamide using sodium hydride as a base, 244 mg (0.46 mmol, 32%) of the crude title compound was isolated.

MS m/z 212 (M+H)⁺.

The following compounds were synthesized according to the reference example 4:

### REFERENCE EXAMPLE 25

### (1Z)-N′-cyano-N-ethylpropanimidamide

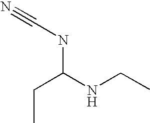

From 1.0 g (7.93 mmol) ethyl (1Z)-N-cyanopropanimidoate, 1.39 g (11.1 mmol, 140%) of the crude (purity ca. 70%) title compound was isolated.

¹H NMR (400 MHz, CDCl₃) δ 3.53-3.43 (m, 1H), 3.18-3.08 (m, 2H), 2.45-2.35 (m 2H), 1.15-1.07 (m, 3H), 1.05-0.95 (m, 3H).

### REFERENCE EXAMPLE 26

### (1Z)-N′-cyano-N-isopropylpropanimidamide

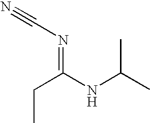

From 1.0 g (7.93 mmol) ethyl (1Z)-N-cyanopropanimidoate, 1.07 g (7.71 mmol, 97%) of the crude title compound was isolated.

¹H NMR (400 MHz, CDCl₃) δ 6.80-6.60 (br, 1H), 4.10-3.98 (m, 1H), 2.54 (q, 2H), 1.25 (t, 3H), 1.16 (d, 6H).

### REFERENCE EXAMPLE 27

### Synthesis of 1-(4-chlorobenzyl)-7-(4-fluorophenyl)-8-methoxy-3,7-dihydro-1H-purine-2,6-dione

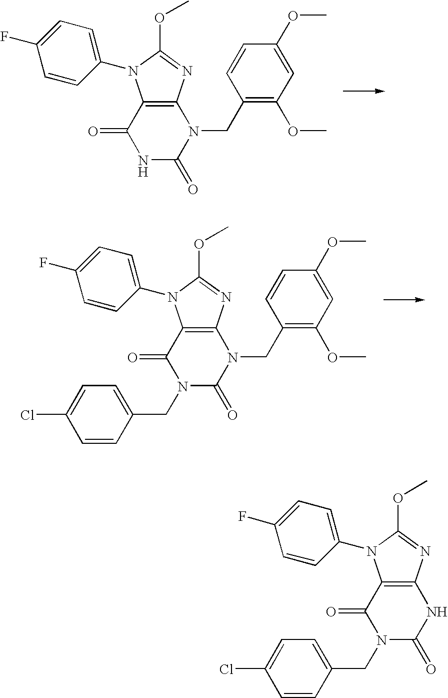

216 mg (0.50 mmol) 3-(2,4-dimethoxybenzyl)-7-(4-fluorophenyl)-8-methoxy-3,7-dihydro-1H-purine-2,6-dione were dissolved in 4 mL DMF, then 325 mg (1 mmol) Cs₂CO₃ were added and after stirring the reaction mixture at room temperature, 123 mg (0.6 mmol) of 4-chlorobenzyl bromide were added. The reaction mixture was stirred at room temperature for 2 h, then partitioned between ethyl acetate and water. The organic layer was washed with water twice, then dried over Na₂SO₄ and evaporated. Crude 1-(4-chlorobenzyl)-3-(2,4-dimethoxybenzyl)-7-(4-fluorophenyl)-8-methoxy-3,7-dihydro-1H-purine-2,6-dione (296 mg) was used without further purification.

276 mg (0.45 mmol, crude) 1-(4-chlorobenzyl)-3-(2,4-dimethoxybenzyl)-7-(4-fluorophenyl)-8-methoxy-3,7-dihydro-1H-purine-2,6-dione were dissolved in 5 mL trifluoroacetic acid, then 262 mg (2.25 mmol) triethylsilane was added. To the biphasic mixture was added 1 mL DCM. The reaction mixture was stirred at room temperature for 48 h, then the solvents were evaporated. The residue was recrystallized from hexanes/ethyl acetate. 89 mg (0.222 mmol, 49%) of 1-(4-chlorobenzyl)-7-(4-fluorophenyl)-8-methoxy-3,7-dihydro-1H-purine-2,6-dione was isolated as colorless solid.

¹H NMR (500 MHz, CDCl₃) δ 9.63 (s, 1H), 7.39 (d, 8.5 Hz, 2H), 7.32-7.37 (m, 2H), 7.21 (d, 8.5 Hz, 2H), 7.12-7.17 (m, 2H), 5.06 (s, 2H), 4.15 (s, 3H).

MS m/z 400.9 (M+H)⁺.

### REFERENCE EXAMPLE 28

### Synthesis of 3-(2,4-dimethoxybenzyl)-7-(4-fluorophenyl)-8-methoxy-3,7-dihydro-1H-purine-2,6-dione

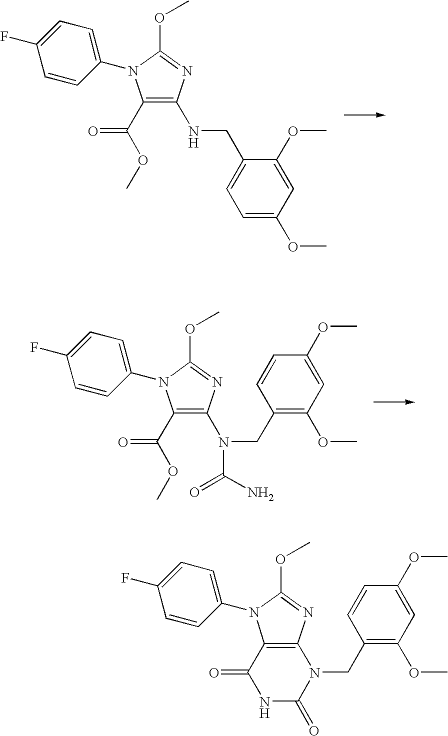

831 mg (2.0 mmol) methyl 4-[(2,4-dimethoxybenzyl)amino]-1-(4-fluorophenyl)-2-methoxy-1H-imidazole-5-carboxylate were dissolved in 5 mL dimethoxyethane, then 1.0 g (9.9 mmol) N-(methoxycarbonyl)isocyanate was added. The resulting mixture was heated in a sealed vial to 130° C. for 13 h using microwave heating. The solvents were evaporated, the residue dissolved in dichloromethane and washed with sat. NaHCO₃. The organic layer was dried by filtration through a phase separator and evaporated.

The residue was dissolved in 8 mL tert-butanol and 40 mg (0.41 mmol) sodium tert-butoxide was added. The reaction mixture was heated to 90° C. for 2 h, then cooled to room temperature. 40 μl (0.7 mmol) of acetic acid was added, the solvents were evaporated and the residue purified by HPFC. 354 mg (0.788 mmol, 39%) 3-(2,4-dimethoxybenzyl)-7-(4-fluorophenyl)-8-methoxy-3,7-dihydro-1H-purine-2,6-dione was isolated as a solid.

¹H NMR (500 MHz, CDCl₃) δ 7.86 (s, 1H), 7.39-7.42 (m, 2H), 7.14-7.18 (m, 2H), 7.11 (d, 8.3 Hz, 1H), 6.49 (d, 2.4 Hz, 1H), 6.45 (dd, 8.4 Hz, 2.4 Hz, 1H), 5.24 (s, 2H), 4.10 (s, 3H), 3.87 (s, 3H), 3.81 (s, 3H).

MS m/z 427.0 (M+H)⁺.

### REFERENCE EXAMPLE 29

### Synthesis of methyl 4-[(2,4-dimethoxybenzyl)amino]-1-(4-fluorophenyl)-2-methoxy-1H-imidazole-5-carboxylate

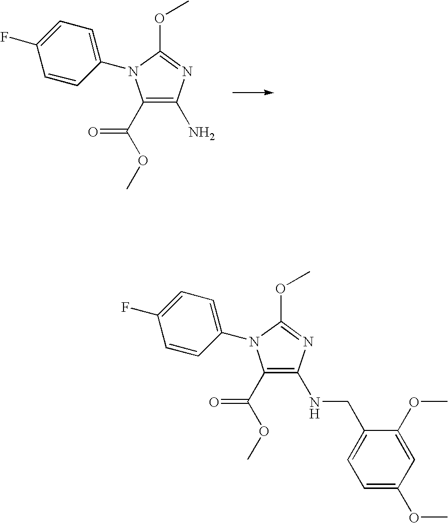

1.05 g (3.95 mmol) methyl 4-amino-1-(4-fluorophenyl)-2-methoxy-1H-imidazole-5-carboxylate were dissolved in 15 ml 1,2-dichloroethane and 1.13 ml (19.78 mmol) acetic acid and 1.5 g (9.02 mmol) 2,4-dimethoxybenzaldehye were added. After 5 min 1.26 g (5.94 mmol) sodium triacetoxyborohydride was added and the resulting mixture stirred overnight at rt. Ethyl acetate was added and washed with sat. NaHCO₃ twice. The organic layer was dried over Na₂SO₄ and evaporated. The residue was purified by flash chromatography on silica using hexanes/ethyl acetate 7:3 as eluent. 1.6 g (3.85 mmol, 97%) of the title compound was isolated.

MS m/z 415.9 (M+H)⁺.

### REFERENCE EXAMPLE 30

### Synthesis of methyl 4-amino-1-(4-fluorophenyl)-2-methoxy-1H-imidazole-5-carboxylate

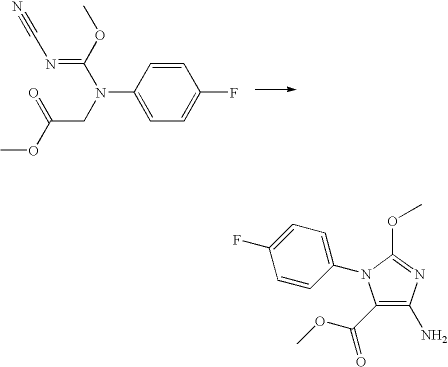

314 mg (1.18 mmol) methyl N—[(cyanoimino)(methoxy)methyl]-N-(4-fluorophenyl) glycinate were dissolved in 10 mL methanol, then 2.5 mL of 0.5 M NaOMe in MeOH (1.25 mmol) were added and the resulting mixture was heated under reflux overnight.

After cooling to room temperature, 75 μL (1.32 mmol) acetic acid were added and the solvents evaporated. The residue was dissolved in ethyl acetate, washed with sat. NaHCO₃, the organic layer dried over MgSO₄ and evaporated. 268 mg (1.01 mmol, 85%) methyl 4-amino-1-(4-fluorophenyl)-2-methoxy-1H-imidazole-5-carboxylate was isolated as a crude brown oil that solidified upon standing. Used without further purification.

¹H-NMR (400 MHz, CDCl₃) δ 7.20-7.16 (m, 2H), 7.08-7.00 (m, 2H), 5.09 (br, 2H), 3.93 (s, 3H), 3.58 (s, 3H).

MS m/z 266 (M+H)⁺.

### REFERENCE EXAMPLE 31

### Synthesis of methyl N-[(cyanoimino)(methoxy)methyl]-N-(4-fluorophenyl) glycinate

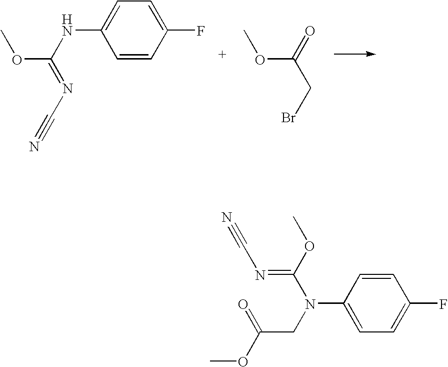

1.46 g (7.56 mmol) methyl N′-cyano-N-(4-fluorophenyl)imidocarbamate were dissolved in 10 mL DMF, then 1.59 g (11.5 mmol) K₂CO₃ and 85 mg (0.23 mmol) tetrabutylammonium iodide were added. After stirring the reaction mixture for 5 min at room temperature 900 μL (9.5 mmol) methyl bromoacetate were added in one portion and the resulting reaction mixture was stirred at room temperature for 1 h. The reaction mixture was partitioned between water and ethyl acetate and after phase separation the organic layer was washed with brine, dried over MgSO₄ and evaporated. 1.98 g (7.46 mmol, 99%) methyl N-[(cyanoimino)(methoxy)methyl]-N-(4-fluorophenyl) glycinate was isolated as a crude brown oil, that solidified upon standing. Used without further purification.

¹H-NMR (400 MHz, CDCl₃) δ 7.38-7.34 (m, 2H), 7.13-7.07 (m, 2H), 4.34 (s, 2H), 3.90 (s, 3H), 3.76 (s, 3H).

MS m/z 266 (M+H)⁺.

### REFERENCE EXAMPLE 32

### Synthesis of methyl N′-cyano-N-(4-fluorophenyl)imidocarbamate

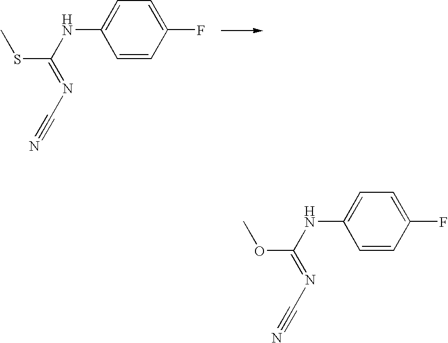

2.09 g (10 mmol) methyl N′-cyano-N-(4-fluorophenyl)imidothiocarbamate was suspended in 30 mL 0.5 M NaOMe in MeOH (15 mmol) and the resulting mixture was heated under reflux for 6 h 30 min. After cooling to room temperature 860 μl (15 mmol) acetic acid were added to the reaction mixture, then the solvents were evaporated until a precipitate started to form (ca. 10-15 mL MeOH left). Then 40 mL of water was added. The precipitate was collected, washed with water and dried. 1.65 g (8.54 mmol, 85%) methyl N′-cyano-N-(4-fluorophenyl)imidocarbamate was isolated as off-white solid.

¹H-NMR (400 MHz, DMSO-d₆) δ 10.2 (s, 1H), 7.31-7.26 (m, 2H), 7.19-7.12 (m, 2H), 3.78 (s, 3H).

MS m/z 194.1 (M+H)⁺.

### REFERENCE EXAMPLE 33

### Synthesis of methyl N′-cyano-N-(4-fluorophenyl)imidothio-carbamate

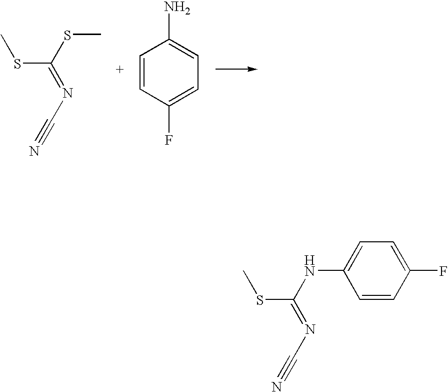

7.31 g (50 mmol) dimethyl N-cyanoiminodithiocarbonate and 5.56 g (50 mmol) 4-fluoroaniline were dissolved in 100 mL abs. ethanol and heated under reflux overnight. The reaction mixture was cooled to room temperature, then 80 mL of hexanes were added under stirring and then cooled in an ice-bath and stirred for 20 min. The formed precipitate was collected and washed with hexanes, then dried. 8.25 g (39.4 mmol, 79%) methyl N′-cyano-N-(4-fluorophenyl)imidothiocarbamate was isolated as a purple solid.

¹H-NMR (400 MHz, DMSO-d₆) δ 10.1 (s, 1H), 7.45-7.40 (m, 2H), 7.23-7.16 (m, 2H), 2.65 (s, 3H).

The following compounds were synthesized according to the reference example 28:

### REFERENCE EXAMPLE 34

### 8-methoxy-7-methyl-3-propyl-3,7-dihydro-1H-purine-2,6-dione

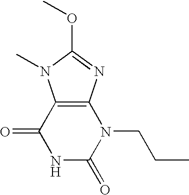

From 1.189 g (5.23 mmol) Methyl 2-methoxy-1-methyl-4-(propylamino)-1H-imidazole-5-carboxylate, 560 mg (2.35 mmol, 45%) of the title compound was isolated.

¹H-NMR (500 MHz, CDCl₃) δ 8-17-8.07 (br, 1H) 4.14 (s, 3H), 3.98 (t, 2H), 3.68 (s, 3H), 1.84-1.74 (m, 2H), 0.97 (t, 3H)

MS m/z 239.1128 (M+H)⁺

### REFERENCE EXAMPLE 35

### 8-methoxy-7-methyl-3-(3,3,3-trifluoropropyl)-3,7-dihydro-1H-purine-2,6-dione

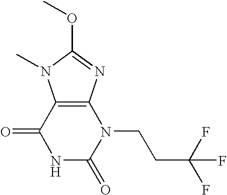

From 100 mg (0.36 mmol) methyl 2-methoxy-1-methyl-4-[(3,3,3-trifluoropropyl)amino]-1H-imidazole-5-carboxylate, 92 mg (0.31 mmol, 86%) of the title compound was isolated.

MS m/z 293 (M+H)⁺.

### REFERENCE EXAMPLE 36

### 8-methoxy-7-methyl-3-(4,4,4-trifluorobutyl)-3,7-dihydro-1H-purine-2,6-dione

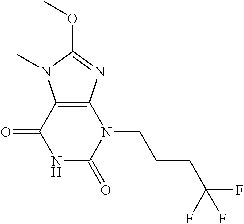

From 254 mg (0.86 mmol) methyl 2-methoxy-1-methyl-4-[(4,4,4-trifluorobutyl)amino]-1H-imidazole-5-carboxylate, 262 mg (0.86 mmol, 100%) of the crude title compound was isolated.

MS m/z 307 (M+H)⁺.

The following compounds were synthesized according to the reference example 29:

### REFERENCE EXAMPLE 37

### Methyl 2-methoxy-1-methyl-4-(propylamino)-1H-imidazole-5-carboxylate

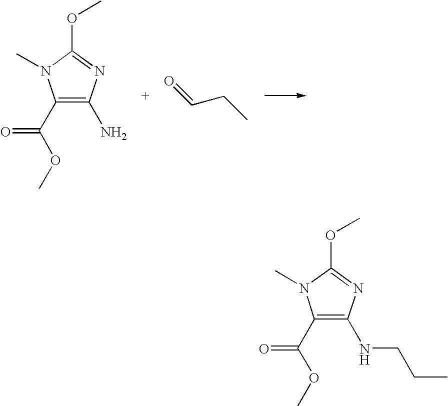

From 900 mg (4.86 mmol) methyl 4-amino-2-methoxy-1-methyl-1H-imidazole-5-carboxylate using dichloromethane as solvent, 1.189 g (107%) of the crude title compound was isolated.

MS m/z 228.1 (M+H)

### REFERENCE EXAMPLE 38

### Methyl 2-methoxy-1-methyl-4-[(3,3,3-trifluoropropyl)amino]-1H-imidazole-5-carboxylate

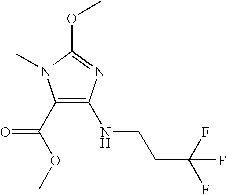

From 253 mg (1.37 mmol) methyl 2-methoxy-1-methyl-4-(propylamino)-1H-imidazole-5-carboxylate, 195 mg (0.69 mmol, 51%) of the title compound was isolated.

¹H NMR (400 MHz, CDCl₃) δ 6.40-5.00 (br, 1H), 3.97 (s, 3H), 3.73 (s, 3H), 3.61 (q, 2H), 3.43 (s, 3H), 2.48-2.34 (m, 2H).

MS m/z 282 (M+H)⁺.

### REFERENCE EXAMPLE 39

### Methyl 2-methoxy-1-methyl-4-[(4,4,4-trifluorobutyl)amino]-1H-imidazole-5-carboxylate

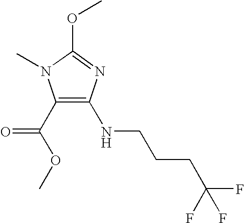

From 187 mg (1.01 mmol) methyl 2-methoxy-1-methyl-4-(propylamino)-1H-imidazole-5-carboxylate, 254 mg (0.86 mmol, 85%) of the title compound was isolated.

¹³C NMR (100 MHz, DMSO-d₆) δ 159.3, 150.2, 145.4, 137.9, 132.1, 130.1, 128.8, 96.3, 44.3, 43.5, 21.5, 11.4.

MS m/z 309.2 (M+H)⁺

### REFERENCE EXAMPLE 44

### Synthesis of 6-amino-3-(4-chlorobenzyl)-5-nitroso-1-propylpyrimidine-2,4(1H,3H)-dione

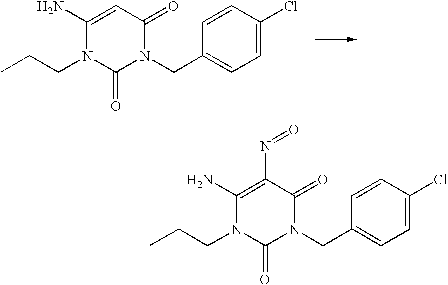

4.89 g (16.65 mmol) 6-amino-3-(4-chlorobenzyl)-1-propylpyrimidine-2,4(1H,3H)-dione was dissolved in 75 mL acetic acid at 80° C. Then NaNO₂ in 10 mL water was added dropwise. After ca. 1 min a thick purple slurry resulted. Heating was continued for 30 min, then addition of ca. 200 mL water and this mixture warmed to 80° C. for 30 min, then cooled in an ice-bath. The bright purple precipitate was collected and washed with water, then dried. 5.51 g (17.07 mmol, quant.) crude 6-amino-3-(4-chlorobenzyl)-5-nitroso-1-propylpyrimidine-2,4(1H,3H)-dione was isolated as pink-purple solid that was used without further purification.

¹H NMR (400 MHz, DMSO-d₆) δ 13.11 (s, 1H), 9.14 (s, 1H), 7.31-7.39 (m, 4H), 5.03 (s, 2H), 3.71-3.76 (m, 2H), 1.43-1.53 (m, 2H), 0.83 (t, 7.3 Hz, 3H).

¹³C NMR (100 MHz, DMSO-d₆) δ 160.7, 149.9, 146.2, 139.7, 136.7, 132.4, 130.2, 128.9, 44.1, 43.4, 20.3, 11.3.

MS m/z 323.2 (M+H)⁺

¹H NMR (400 MHz, CDCl₃) δ 6.40-5.00 (br, 1H), 3.95 (s, 3H), 3.71 (s, 3H), 3.45-3.36 (br, 5H), 2.19-2.05 (m, 2H), 1.85-1.75 (m, 2H).

MS m/z 296 (M+H)⁺.

The following compound was synthesized according to the reference examples 33-30:

### REFERENCE EXAMPLE 40

### Methyl 4-amino-2-methoxy-1-methyl-1H-imidazole-5-carboxylate

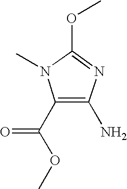

From 250 g (1.712 mol) dimethyl N-cyanoiminodithiocarbonate, 41.8 g (0.226 mol, 13.2%) methyl 4-amino-2-methoxy-1-methyl-1H-imidazole-5-carboxylate was isolated as a colorless solid.

¹H-NMR (400 MHz, CDCl₃) δ 4.84 (s, 2H), 3.96 (s, 3H), 3.76 (s, 3H), 3.45 (s, 3H).

MS m/z 186.0 (M+H)⁺.

### REFERENCE EXAMPLE 41

### Synthesis of 1-(4-chlorobenzyl)-3-propyl-3,7-dihydro-1H-purine-2,6-dione

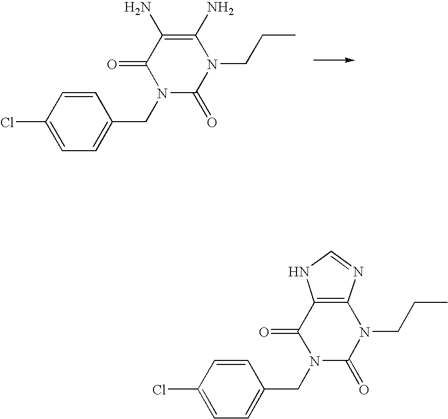

747 mg (2.42 mmol) of 5,6-diamino-3-(4-chlorobenzyl)-1-propylpyrimidine-2,4(1H,3H)-dione was suspended in triethylorthoformate and heated to 120° C. for 90 min. The solvents were evaporated and the residue dried in vacuum. 810 mg (2.41 mmol, quant.) crude 1-(4-chlorobenzyl)-3-propyl-3,7-dihydro-1H-purine-2,6-dione was isolated as off-white solid that was used without further purification.

¹H NMR (400 MHz, DMSO-d₆) δ 13.57 (s, 1H), 8.03 (s, 1H), 7.31 (d, 8.5 Hz, 2H), 7.26 (d, 8.5 Hz, 2H), 5.01 (s, 2H), 3.90-3.95 (m, 2H), 1.61-1.68 (m, 2H), 0.82 (t, 7.5 Hz, 3H).

MS m/z 363.2 (M+H)⁺

### REFERENCE EXAMPLE 42

### Synthesis of 1-(4-chlorobenzyl)-8-(1-hydroxyethyl)-3-propyl-3,7-dihydro-1H-purine-2,6-dione

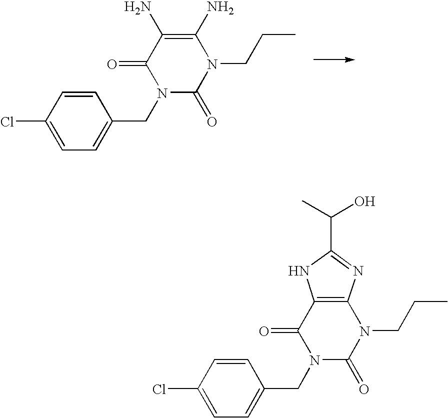

100 mg (0.291 mmol) of 5,6-diamino-3-(4-chlorobenzyl)-1-propylpyrimidine-2,4(1H,3H)-dione was mixed with 66 mg (0.622 mmol) 2-hydroxypropionic acid in 1 mL dioxane and heated to 100° C. for 1 h in a sealed vial using microwave heating. The reaction mixture was transferred into 1 mL of a 1:1 mixture of water and ethanol and 58 mg (1.46 mmol) NaOH was added. The resulting mixture was heated under reflux for 90 min. The reaction mixture was acidified by the addition of acetic acid and cooled to room temperature, then diluted with water. This mixture was extracted with dichloromethane twice. The combined organic layers were dried over MgSO₄ and evaporated. The residue was purified by reversed phase HPLC. 60 mg (0.165 mmol, 57%) 1-(4-chlorobenzyl)-8-(1-hydroxyethyl)-3-propyl-3,7-dihydro-1H-purine-2,6-dione was isolated as a yellowish solid.

¹H NMR (500 MHz, CD₃OD) δ 7.29 (d, 8.5 Hz, 2H), 7.19 (d, 8.5 Hz, 2H), 5.08 (s, 2H), 4.91 (q, 6.6 Hz, 1H), 4.82 (br. s, 2H) 3.96-4.01 (m, 2H), 1.67-1.74 (m, 2H), 1.51 (d, 6.6 Hz, 3H), 0.89 (t, 7.5 Hz, 3H).

MS m/z 363.2 (M+H)⁺

### REFERENCE EXAMPLE 43

### Synthesis of 5,6-diamino-3-(4-chlorobenzyl)-1-propylpyrimidine-2,4(1H,3H)-dione

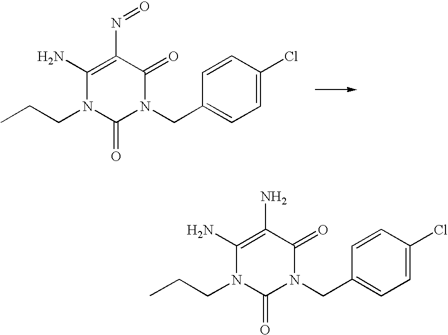

2.33 g (7.22 mmol) 6-amino-3-(4-chlorobenzyl)-5-nitroso-1-propylpyrimidine-2,4(1H,3H)-dione was suspended in acetonitrile, then 60 mL of ca. 13% aq. ammonia was added and an orange solution resulted. This solution was warmed to 80° C. in an oil-bath. Then 2.51 g (14.44 mmol) sodium dithionite was added as a solid in portions over 5 min. and the resulting mixture stirred at 80° C. for 1 h. The reaction mixture was cooled to room temperature, reduced to half its volume (evaporation of acetonitrile) and diluted with more water. The formed solid was collected, washed with water and dried. 1.9 g (6.15 mmol, 85%) 5,6-diamino-3-(4-chlorobenzyl)-1-propylpyrimidine-2,4(1H,3H)-dione was isolated as slightly green solid.

¹H NMR (400 MHz, DMSO-d₆) δ 7.27 (d, 8.3 Hz, 2H), 7.19 (d, 8.3 Hz, 2H), 6.23 (s, 2H), 4.88 (s, 2H), 3.70-3.75 (m, 2H), 2.86 (s, 2H), 1.44-1.50 (m, 2H), 0.78 (t, 7.5 Hz, 3H).

### REFERENCE EXAMPLE 45

### Synthesis of 6-amino-3-(4-chlorobenzyl)-1-propylpyrimidine-2,4(1H,3H)-dione

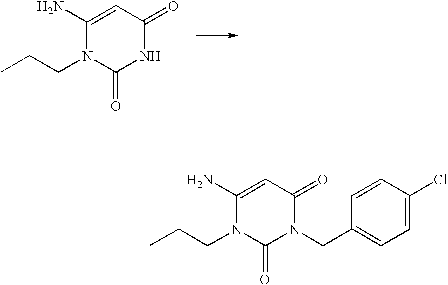

8.46 g (50 mmol) of 6-amino-1-propylpyrimidine-2,4(1H,3H)-dione was suspended in 20 mL DMF, then 6.28 g (52.7 mmol) of dimethylformamide dimethylacetal was added and the resulting mixture warmed to 40° C. for 3 h, then addition of 10 mL DMF and 450 mg (3.8 mmol) of dimethylformamide dimethylacetal. Stirring was continued at 40° C. for additional 30 min, then 12.33 g (60 mmol) of 4-chlorobenzyl bromide and 13.82 g (100 mmol) of K₂CO₃ were added in one portion followed by 10 mL DMF.

The reaction temperature was increased to 80° C. and after 1 h additional 3.08 g (15 mmol) of 4-chlorobenzyl bromide and 15 mL DMF were added. The reaction mixture was stirred for three days at 80° C., then cooled to room temperature. 150 mL ethyl acetate was added to the reaction mixture and then filtered. The solids were washed with ethyl acetate. The combined filtrated were evaporated. The solid residue was suspended in methanol and sonicated until a fine suspension resulted. The solid was collected and washed with methanol. From the combined filtrates additional solid could be isolated using the same procedure. 6.94 g of colorless solid was isolated. To two portions (5.04 g and 1.9 g) of this solid was added 5 mL MeOH and 15 mL cone. aq. NH₃ each and the resulting mixtures were heated in sealed vials for 1 h to 120° C. using microwave heating. The two reaction mixtures were combined, the solvents evaporated and the residue crystallized from methanol/water. 4.93 g (16.78 mmol, 33%) of 6-amino-3-(4-chlorobenzyl)-1-propylpyrimidine-2,4(1H,3H)-dione was isolated as colorless solid.

¹H NMR (400 MHz, DMSO-d₆) δ 7.30 (d, 8.5 Hz, 2H), 7.20 (d, 8.5 Hz, 2H), 6.87 (s, 2H), 4.84 (s, 2H), 4.68 (s, 1H), 3.66-3.71 (m, 2H), 1.42-1.53 (m, 2H), 0.80 (t, 7.4 Hz, 3H).

¹³C NMR (100 MHz, DMSO-d₆) δ 161.7, 155.2, 152.0, 138.0, 132.0, 130.0, 128.8, 75.5, 44.0, 43.0, 21.4, 11.4.

MS m/z 294.1 (M+H)⁺

### REFERENCE EXAMPLE 46

### Synthesis of 6-amino-1-propylpyrimidine-2,4(1H,3H)-dione

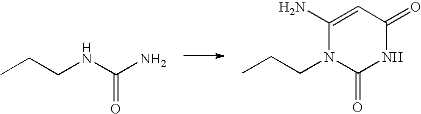

To a mixture of 26.86 g (263 mmol) of 1-propylurea and 30.74 g (271 mmol) of ethyl cyanoacetate was added 150 mL of a 21% solution of sodium ethoxide in ethanol. The resulting mixture was heated under reflux with exclusion of moisture (drying tube on reflux condenser) overnight. The reaction mixture was cooled to room temperature and the solvents evaporated. The residue was dissolved in 200 mL water and heated for 2 h under reflux. After cooling to room temperature, conc. HCl was added slowly until pH=4. A precipitate formed. The resulting suspension was stirred at room temperature overnight, then the solid was collected, washed with water and dried. 23.23 g (137.3 mmol, 52%) of 6-amino-1-propylpyrimidine-2,4(1H,3H)-dione was isolated as a beige solid that was used without further purification.

¹H NMR (400 MHz, DMSO-d₆) δ 10.24 (s, 1H), 6.73 (s, 2H), 4.48 (s, 1H), 3.29-3.66 (m, 2H), 1.42-1.52 (m, 2H), 0.81 (t, 7.5 Hz, 3H).

### REFERENCE EXAMPLE 47

### Synthesis of 1-propylurea

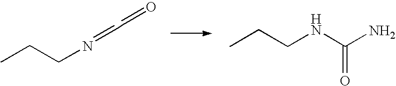

To 100 mL of a 2 M solution of ammonia in ethanol (200 mmol) was added 7 mL (73.7 mmol) propylisocyanate. The resulting mixture was stirred at room temperature for 30 min, then the solvents were evaporated. The residue was dried to give 7.44 g (72.8 mmol, 99%) of 1-propylurea as a colorless solid that was used without further purification.

¹H NMR (400 MHz, DMSO-d₆) δ 5.86 (s, 1H), 5.31 (s, 2H), 2.83-2.89 (m, 2H), 1.26-1.36 (m, 2H), 0.78 (t, 7.5 Hz, 3H).

¹³C NMR (100 MHz, DMSO-d₆) δ 159.4, 41.7, 23.8, 12.0.

### EXAMPLES

Examples 3, 5, 17, 45, 53, 55, 65, 73, 90, and 97-103 are for comparative purposes only.

### Example 1

### Synthesis of 3-benzyl-1-(4-chlorobenzyl)-8-ethyl-7-methyl-3,7-dihydro-1H-purine-2,6-dione

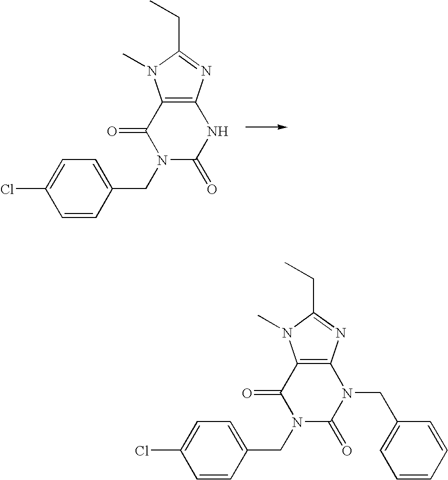

30 mg (0.094 mmol) 1-(4-chlorobenzyl)-8-ethyl-7-methyl-3,7-dihydro-1H-purine-2,6-dione was dissolved in 1 mL DMF, then 92 mg (0.282 mmol) Cs₂CO₃, followed by 40 mg (0.234 mmol) benzyl bromide. The reaction mixture was stirred for 90 min at rt, then partitioned between water and ethyl acetate. The organic layer was dried by filtration through a phase separator and evaporated. The residue was purified by preparative HPLC and 21 mg (0.049 mmol, 52%) of the title compound was isolated.

¹H-NMR (400 MHz, CDCl₃) δ 7.55-7.49 (m, 2H), 7.44-7.40 (m, 2H), 7.34-7.23 (m, 5H), 5.27 (s, 2H), 5.14 (s, 2H), 3.90 (s, 3H), 2.77 (q, 2H), 1.37 (t, 3H)

MS m/z 409.1438 (M+H)⁺

The following compounds were synthesized according to example 1:

### Example 2

### 1-(4-Chlorobenzyl)-3-(3,3-dimethylbutyl)-8-ethyl-7-methyl-3,7-dihydro-1H-purine-2,6-dione

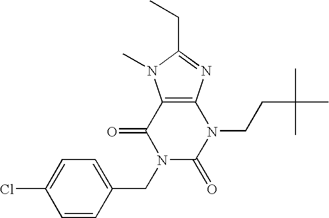

From 76 mg (0.24 mmol) 1-(4-chlorobenzyl)-8-ethyl-7-methyl-3,7-dihydro-1H-purine-2,6-dione using potassium carbonate as base, 87 mg (0.199 mmol, 83%) of the title compound was isolated.

¹H-NMR (400 MHz, CDCl₃) δ 7.42-7.35 (m, 2H), 7.26-7.18 (m, 2H), 5.11 (s, 2H), 4.13-4.02 (m, 2H), 3.86 (s, 3H), 2.71 (q, 2H), 1.64-1.54 (m, 2H), 1.31 (t, 3H), 0.98 (s, 9H)

MS m/z 403.1902 (M+H)⁺

### Example 3

### Tert-butyl {2-[1-(4-chlorobenzyl)-8-ethyl-7-methyl-2,6-dioxo-1,2,6,7-tetrahydro-3H-purin-3-yl]ethyl}carbamate

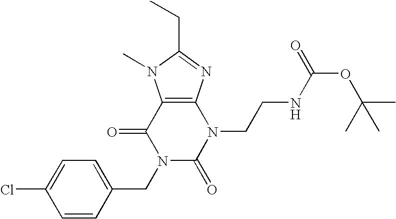

From 60 mg (0.188 mmol) 1-(4-chlorobenzyl)-8-ethyl-7-methyl-3,7-dihydro-1H-purine-2,6-dione, 44 mg (0.093 mmol, 50%) of the title compound was isolated.

¹H-NMR (400 MHz, CDCl₃) δ 7.32-7.24 (m, 2H), 7.19-7.05 (m, 2H), 5.01 (s, 2H), 4.16-4.05 (m, 2H), 3.76 (s, 3H), 3.45-3.27 (m, 2H) 2.62 (q, 2H), 1.28-1.16 (m, 12H)

MS m/z 462.1898 (M+H)⁺

### Example 4

### 1-(4-Chlorobenzyl)-3-(3,3-dimethyl-2-oxobutyl)-8-ethyl-7-methyl-3,7-dihydro-1H-purine-2,6-dione

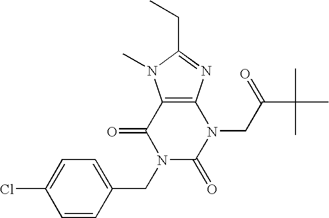

From 300 mg (0.941 mmol) 1-(4-chlorobenzyl)-8-ethyl-7-methyl-3,7-dihydro-1H-purine-2,6-dione using potassium carbonate as base, 213 mg (0.506 mmol, 54%) of the title compound was isolated.

¹H-NMR (500 MHz, CDCl₃) δ 7.29-7.21 (m, 2H), 7.16-7.10 (m, 2H), 5.02 (s, 2H), 4.91 (s, 2H), 3.74 (s, 3H), 2.54 (q, 2H), 1.21-1.11 (m, 12H)

MS m/z 417.1711 (M+H)⁺

### Example 5

### 2-[1-(4-Chlorobenzyl)-8-ethyl-7-methyl-2,6-dioxo-1,2,6,7-tetrahydro-3H-purin-3-yl]acetamide

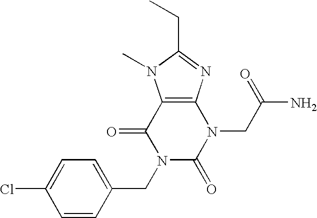

From 50 mg (0.157 mmol) 1-(4-chlorobenzyl)-8-ethyl-7-methyl-3,7-dihydro-1H-purine-2,6-dione using potassium carbonate as base, 18 mg (0.047 mmol, 30%) of the title compound was isolated.

¹H-NMR (400 MHz, DMF-d₇) δ 7.94 (br, 1H), 7.61-7.48 (m, 4H), 7.35 (br, 1H), 5.26 (s, 2H), 4.85 (s, 2H), 4.06 (s, 3H) 2.94 (q, 2H), 1.40 (t, 3H)

MS m/z 376.1166 (M+H)⁺

### Example 6

### 1-(4-Chlorobenzyl)-8-ethyl-7-methyl-3-{[(2R)-5-oxopyrrolidin-2-yl]methyl}-3,7-dihydro-1H-purine-2,6-dione

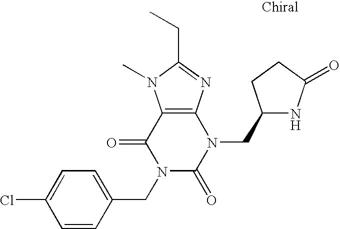

From 50 mg (0.157 mmol) 1-(4-chlorobenzyl)-8-ethyl-7-methyl-3,7-dihydro-1H-purine-2,6-dione using potassium carbonate as base, 42 mg (0.099 mmol, 63%) of the title compound was isolated.

¹H-NMR (600 MHz, (CH₃)₂SO*, (CD₃)₂SO)) δ 7.35-7.27 (m, 4H), 5.00 (s, 2H), 4.05-3.95 (m, 2H), 3.90-3.86 (m, 1H), 3.81 (s, 3H), 2.73 (q, 2H), 2.23-1.99 (m, 3H), 1.79-1.71 (m, 1H), 1.20 (t, 3H)

MS m/z 416.1510 (M+H)⁺

### Example 7

### 1-(4-Chlorobenzyl)-8-ethyl-7-methyl-3-(2-oxo-2-pyridin-4-ylethyl)-3,7-dihydro-1H-purine-2,6-dione

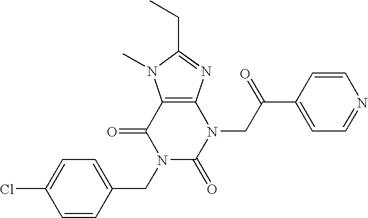

From 50 mg (0.157 mmol) 1-(4-chlorobenzyl)-8-ethyl-7-methyl-3,7-dihydro-1H-purine-2,6-dione using potassium carbonate as base, 30 mg (0.069 mmol, 44%) of the title compound was isolated.

¹H-NMR (600 MHz, (CH₃)₂SO*, (CD₃)₂SO)) δ 8.87-8.82 (m, 2H), 7.95-7.90 (m, 2H), 7.37-7.31 (M, 2H), 7.29-7.24 (m, 2H), 5.04 (s, 2H), 5.02 (s, 2H), 3.82 (s, 3H), 2.69 (q, 2H), 1.14 (t, 3H)

MS m/z 438,1325 (M+H)⁺

### Example 8

### 1-(4-Chlorobenzyl)-8-ethyl-3-isobutyl-7-methyl-3,7-dihydro-1H-purine-2,6-dione

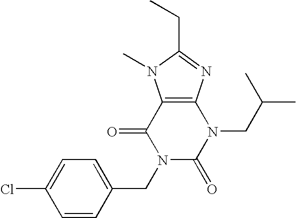

From 50 mg (0.157 mmol) 1-(4-chlorobenzyl)-8-ethyl-7-methyl-3,7-dihydro-1H-purine-2,6-dione using potassium carbonate as base, 31 mg (0.082 mmol, 52%) of the title compound was isolated.

¹H-NMR (600 MHz, (CH₃)₂SO*, (CD₃)₂SO)) δ 7.35-7.30 (m, 2H), 7.29-7.24 (m, 2H), 5.00 (s, 2H), 3.80 (s, 3H), 3.78-3.75 (m, 2H), 2.72 (q, 2H), 2.20-2.11 (m, 1H) 1.14 (t, 3H), 0.85-0.79 (m, 6H)

MS m/z 375.1577 (M+H)⁺

### Example 9

### 1-(4-Chlorobenzyl)-8-ethyl-7-methyl-3-[(trimethylsilyl)methyl]-3,7-dihydro-1H-purine-2,6-dione

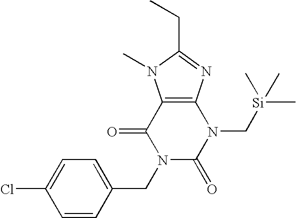

From 50 mg (0.157 mmol) 1-(4-chlorobenzyl)-8-ethyl-7-methyl-3,7-dihydro-1H-purine-2,6-dione using potassium carbonate as base, 34 mg (0.082 mmol, 52%) of the title compound was isolated.

¹H-NMR (400 MHz, CDCl₃) δ 7.40-7.31 (m, 2H), 7.23-7.14 (m, 2H), 5.10 (s, 2H), 3.83 (s, 3H), 3.60 (s, 2H), 2.67 (q, 2H), 1.26 (t, 3H), 0.00 (s, 9H)

MS m/z 405.1518 (M+H)⁺

### Example 10

### 1-(4-Chlorobenzyl)-8-ethyl-7-methyl-3-{[(2S)-5-oxopyrrolidin-2-yl]methyl}-3,7-dihydro-1H-purine-2,6-dione

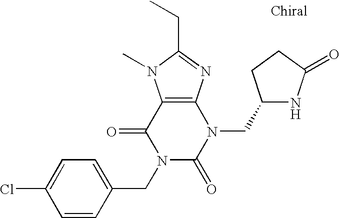

From 48 mg (0.151 mmol) 1-(4-chlorobenzyl)-8-ethyl-7-methyl-3,7-dihydro-1H-purine-2,6-dione using potassium carbonate as base, 30 mg (0.038 mmol, 25%) of 50% pure title compound was isolated.

¹H-NMR (400 MHz, CDCl₃) δ 7.43-7.33 (m, 2H), 7.28-7.15 (m, 2H), 6.54 (br, 1H), 5.17-4.99 (m, 2H), 4.33-4.22 (m, 1H), 4.14-3.96 (m, 2H), 3.90-3.78 (m, 3H), 2.85-2.62 (m, 2H), 2.38-2.17 (m, 2H) 2.02 (br, 1H), 2.00-1.88 (m, 1H) 1.39-1.21 (m, 3H)

MS m/z 416.1480 (M+H)⁺

### Example 11

### Methyl [1-(4-chlorobenzyl)-8-ethyl-7-methyl-2,6-dioxo-1,2,6,7-tetrahydro-3H-purin-3-yl]acetate

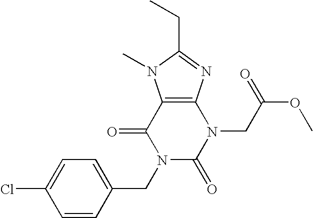

From 508 mg (1.59 mmol) 1-(4-chlorobenzyl)-8-ethyl-7-methyl-3,7-dihydro-1H-purine-2,6-dione using potassium carbonate as base, 610 mg (1.56 mmol, 98%) of crude title compound was isolated.

¹H-NMR (400 MHz, CDCl₃) δ 7.40-7.33 (m, 2H), 7.26-7.20 (m, 2H), 5.12 (s, 2H), 4.79 (s, 2H) 3.87 (s, 3H), 3.75 (s, 3H), 2.69 (q, 2H), 1.28 (t, 3H)

MS m/z 391.2 (M+H)⁺

### Example 12

### 3-Allyl-1-(4-chlorobenzyl)-8-ethyl-7-methyl-3,7-dihydro-1H-purine-2,6-dione

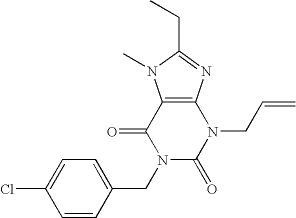

From 407 mg (1.28 mmol) 1-(4-chlorobenzyl)-8-ethyl-7-methyl-3,7-dihydro-1H-purine-2,6-dione using potassium carbonate as base, 273 mg (0.75 mmol, 58%) of crude title compound was isolated.

¹H-NMR (500 MHz, CDCl₃) δ 7.46-7.40 (m, 2H), 7.29-7.24 (m, 2H), 6.03-5.93 (m, 1H), 5.29-5.19 (m, 2H), 5.16 (s, 2H), 4.70 (d, 2H), 3.93 (s, 3H), 2.76 (q, 2H), 1.34 (t, 3H)

MS m/z 359.1275 (M+H)⁺

### Example 13

### 1,3-Bis(4-chlorobenzyl)-8-ethyl-7-methyl-3,7-dihydro-1H-purine-2,6-dione

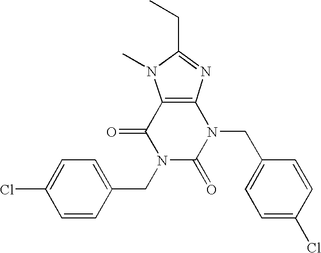

From 31.9 mg (0.1 mmol) 1-(4-chlorobenzyl)-8-ethyl-7-methyl-3,7-dihydro-1H-purine-2,6-dione using cesium carbonate as base, 8.6 mg (0.019 mmol, 21.6%) of crude title compound was isolated.

¹H-NMR (600 MHz, (CH₃)₂SO*, (CD₃)₂SO)) δ 7.35-7.28 (m, 6H), 7.26-7.23 (m, 2H), 5.09 (s, 2H), 4.98 (s, 2H), 3.79 (s, 3H), 2.71 (q, 2H), 1.13 (t, 3H)

MS m/z 443,1016 (M+H)⁺

### Example 14

### 1-(4-Chlorobenzyl)-3-(1,3-dioxolan-2-ylmethyl)-8-ethyl-7-methyl-3,7-dihydro-1H-purine-2,6-dione

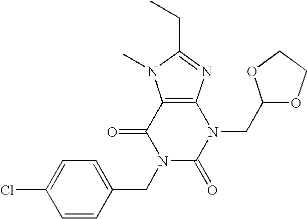

From 150 mg (0.47 mmol) 1-(4-chlorobenzyl)-8-ethyl-7-methyl-3,7-dihydro-1H-purine-2,6-dione using potassium carbonate as base, 20 mg (0.031 mmol, 7%) of the title compound was isolated.

¹H-NMR (400 MHz, CDCl₃) δ 7.42-7.34 (m, 2H), 7.26-7.18 (m, 2H), 5.42 (t, 1H), 5.11 (s, 2H), 4.41-4.31 (m, 1H), 4.19 (d, 2H), 4.07-4.01 (m, 2H) 3.89-3.81 (m, 4H), 2.71 (q, 2H), 1.30 (t, 3H)

MS m/z 405.1326 (M+H)⁺

### Example 15

### 1-(4-Chlorobenzyl)-8-ethyl-7-methyl-3-(pyridin-2-ylmethyl)-3,7-dihydro-1H-purine-2,6-dione

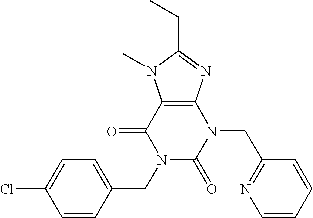

From 31.9 mg (0.1 mmol) 1-(4-chlorobenzyl)-8-ethyl-7-methyl-3,7-dihydro-1H-purine-2,6-dione using cesium carbonate as base, 9.2 mg (0.025 mmol, 24.9%) of crude title compound was isolated.

¹H-NMR (600 MHz, (CH₃)₂SO*, (CD₃)₂SO)) δ 8.43-8.39 (m, 1H), 7.73-7.68 (m, 1H), 7.35-7.30 (m, 2H), 7.28-7.20 (m, 4H), 5.24 (s, 2H), 5.01 (s, 2H), 3.81 (s, 3H), 2.68 (q, 2H), 1.13 (t, 3H)

MS m/z 410.1403 (M+H)⁺

### Example 16

### 1-(4-Chlorobenzyl)-8-ethyl-7-methyl-3-[(5-methylisoxazol-3-yl)methyl]-3,7-dihydro-1H-purine-2,6-dione

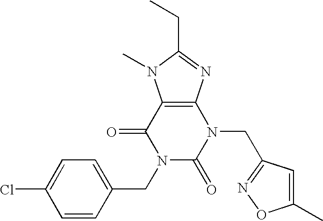

From 31.9 mg (0.1 mmol) 1-(4-chlorobenzyl)-8-ethyl-7-methyl-3,7-dihydro-1H-purine-2,6-dione using cesium carbonate as base, 13.9 mg (0.037 mmol, 37.3%) of crude title compound was isolated.

¹H-NMR (600 MHz, (CH₃)₂SO*, (CD₃)₂SO)) δ 7.34-7.30 (m, 2H), 7.28-7.23 (m, 2H), 6.10 (s, 1H), 5.13 (s, 2H), 5.00 (s, 2H), 3.80 (s, 3H), 2.72 (q, 2H), 2.31 (s, 3H), 1.18 (t, 3H)

MS m/z 414.1352 (M+H)⁺

### Example 17

### 1-(4-Chlorobenzyl)-3-{[1-(4-chlorophenyl)-5-(trifluoromethyl)-1H-pyrazol-4-yl]methyl}-8-ethyl-7-methyl-3,7-dihydro-1H-purine-2,6-dione

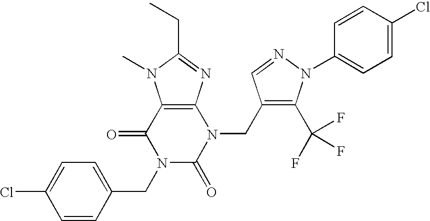

From 31.9 mg (0.1 mmol) 1-(4-Chlorobenzyl)-8-ethyl-7-methyl-3,7-dihydro-1H-purine-2,6-dione using cesium carbonate as base, 25.3 mg (0.049 mmol, 48.7%) of crude title compound was isolated.

¹H-NMR (600 MHz, (CH₃)₂SO*, (CD₃)₂SO)) δ 7.65 (s, 1H), 7.62-7.59 (m, 2H), 7.49-7.46 (m, 2H), 7.35-7.27 (m, 4H), 5.21 (s, 2H), 5.02 (s, 2H), 3.81 (s, 3H), 2.73 (q, 2H), 1.20 (t, 3H)

MS m/z 577.1131 (M+H)⁺

### Example 18

### 1-(4-Chlorobenzyl)-8-ethyl-7-methyl-3-(pyridin-3-ylmethyl)-3,7-dihydro-1H-purine-2,6-dione

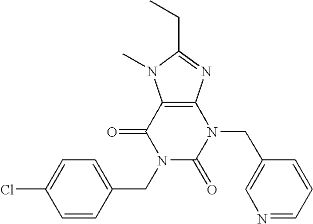

From 31.9 mg (0.1 mmol) 1-(4-chlorobenzyl)-8-ethyl-7-methyl-3,7-dihydro-1H-purine-2,6-dione using cesium carbonate as base, 11 mg (0.030 mmol, 29.8%) of crude title compound was isolated.

¹H-NMR (600 MHz, (CH₃)₂SO*, (CD₃)₂SO)) δ 8.54 (s, 1H), 8.45-8.41 (m, 1H), 7.72-7.68 (m, 1H), 7.33-7.29 (m, 3H), 7.28-7.24 (m, 2H), 5.14 (s, 2H), 4.98 (s, 2H), 3.79 (s, 3H), 2.73 (q, 2H), 1.21 (t, 3H)

MS m/z 410.1378 (M+H)⁺

### Example 19

### 1-(4-Chlorobenzyl)-3-[4-(difluoromethoxy)benzyl]-8-ethyl-7-methyl-3,7-dihydro-1H-purine-2,6-dione

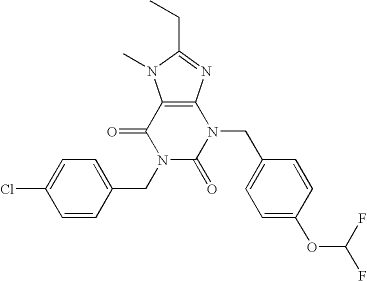

From 31.9 mg (0.1 mmol) 1-(4-chlorobenzyl)-8-ethyl-7-methyl-3,7-dihydro-1H-purine-2,6-dione using cesium carbonate as base, 18.1 mg (0.042 mmol, 42.3%) of crude title compound was isolated.

¹H-NMR (600 MHz, (CH₃)₂SO*, (CD₃)₂SO)) δ 7.38-7.30 (m, 4H), 7.28-7.23 (m, 2H), 7.13 (t, 1H), 7.10-7.06 (m, 2H), 5.14 (s, 2H), 4.98 (s, 2H), 3.79 (s, 3H), 2.73 (q, 2H), 1.21 (t, 3H)

MS m/z 475.1369 (M+H)⁺

### Example 20

### 1-(4-Chlorobenzyl)-3-(cyclohexylmethyl)-8-ethyl-7-methyl-3,7-dihydro-1H-purine-2,6-dione

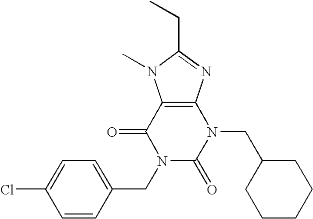

From 31.9 mg (0.1 mmol) 1-(4-chlorobenzyl)-8-ethyl-7-methyl-3,7-dihydro-1H-purine-2,6-dione using cesium carbonate as base, 15.7 mg (0.042 mmol, 42.0%) of crude title compound was isolated.

¹H-NMR (600 MHz, (CH₃)₂SO*, (CD₃)₂SO)) δ 7.35-7.29 (m, 2H), 7.27-7.21 (m, 2H), 4.99 (s, 2H), 3.82-3.74 (m, 5H), 2.72 (q, 2H), 1.86-1.77 (m, 1H), 1.66-1.57 (m, 2H), 1.57-1.47 (m, 2H), 1.19 (t, 3H), 1.15-0.82 (m, 6H)

MS m/z 415.1901 (M+H)⁺

### Example 21

### 3-(3-tert-butoxypropyl)-1-(4-chlorobenzyl)-8-ethyl-7-methyl-3,7-dihydro-1H-purine-2,6-dione

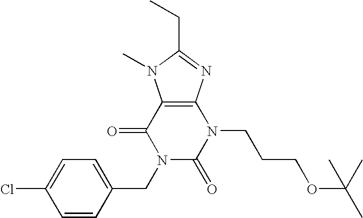

From 31.9 mg (0.1 mmol) 1-(4-chlorobenzyl)-8-ethyl-7-methyl-3,7-dihydro-1H-purine-2,6-dione using potassium carbonate as base, 20.2 mg (0.047 mmol, 47%) of the title compound was isolated.

¹H-NMR (600 MHz, (CH₃)₂SO*, (CD₃)₂SO): δ 7.33-7.26 (m, 4H), 5.00 (s, 2H), 4.02-3.98 (m, 2H), 3.80 (s, 3H), 2.73 (q, 2H), 2.67-2.38 (m, 2H), 1.83-1.77 (m, 2H), 1.21 (t, 3H), 1.00 (s, 9H).

HRMS Calcd for [C₂₂H₂₉ClN₄O₃+H]⁺: 433.2006. Found: 433.1979.

### Example 22

### 1-(4-chlorobenzyl)-8-ethyl-7-methyl-3-[4-(methylsulfonyl)benzyl]-3,7-dihydro-1H-purine-2,6-dione

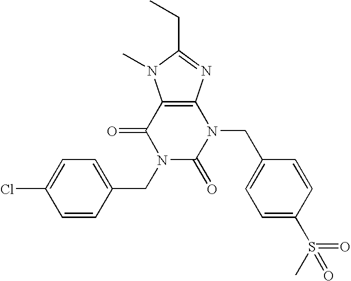

From 31.9 mg (0.1 mmol) 1-(4-chlorobenzyl)-8-ethyl-7-methyl-3,7-dihydro-1H-purine-2,6-dione using potassium carbonate as base, 30.5 mg (0.063 mmol, 63%) of the title compound was isolated.

¹H-NMR (600 MHz, (CH₃)₂SO*, (CD₃)₂SO): δ 7.84 (d, 2H), 7.52 (d, 2H), 7.35-7.26 (m, 4H), 5.23 (s, 2H), 5.01 (s, 2H), 3.82 (s, 3H), 3.16 (s, 3H), 2.73 (q, 2H), 1.19 (t, 3H).

HRMS Calcd for [C₂₃H₂₃ClN₄O₄S+H]⁺: 487.1207. Found: 487.1191.

### Example 23

### 1-(4-chlorobenzyl)-8-ethyl-7-methyl-3-(3,3,3-trifluoro-2-hydroxypropyl)-3,7-dihydro-1H-purine-2,6-dione

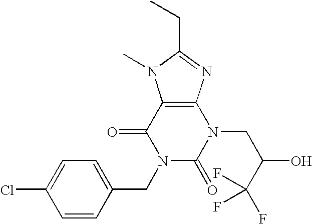

From 31.9 mg (0.1 mmol) 1-(4-chlorobenzyl)-8-ethyl-7-methyl-3,7-dihydro-1H-purine-2,6-dione using potassium carbonate as base, 30.9 mg (0.072 mmol, 72%) of the title compound was isolated.

¹H-NMR (600 MHz, (CH₃)₂SO*, (CD₃)₂SO): δ 7.35-7.26 (m, 4H), 6.54 (d, 1H), 5.01 (dd, 2H), 4.53-4.46 (br, 1H), 4.27-4.21 (m, 1H), 4.12-4.06 (m, 1H), 3.81 (s, 3H), 2.74 (q, 2H), 1.20 (t, 3H).

HRMS Calcd for [C₁₈H₁₈ClF₃N₄O₃+H]⁺: 431.1098. Found: 431.1106.

### Example 24

### 1-(4-chlorobenzyl)-3-(2,3-dihydro-1,4-benzodioxin-2-ylmethyl)-8-ethyl-7-methyl-3,7-dihydro-1H-purine-2,6-dione

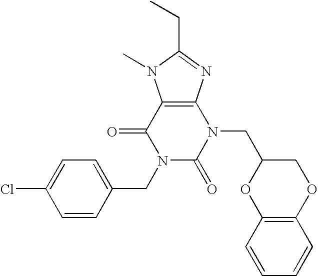

From 31.9 mg (0.1 mmol) 1-(4-chlorobenzyl)-8-ethyl-7-methyl-3,7-dihydro-1H-purine-2,6-dione using potassium carbonate as base, 30.0 mg (0.064 mmol, 64%) of the title compound was isolated.

¹H-NMR (600 MHz, (CH₃)₂SO*, (CD₃)₂SO): δ 7.36-7.27 (m, 4H), 6.85-6.75 (m, 3H), 6.68-6.65 (m, 1H), 5.01 (dd, 2H), 4.59-4.54 (m, 1H), 4.37-4.32 (m, 1H), 4.28-4.24 (m, 1H), 4.17-4.12 (m, 1H), 4.08-4.04 (m, 1H), 3.80 (s, 3H), 2.70 (q, 2H), 1.15 (t, 3H).

HRMS Calcd for [C₂₄H₂₃ClN₄O₄+H]⁺: 467.1486. Found: 467.1479.

### Example 25

### 1-(4-chlorobenzyl)-8-ethyl-7-methyl-3-{4-[(trifluoromethyl)thio]benzyl}-3,7-dihydro-1H-purine-2,6-dione

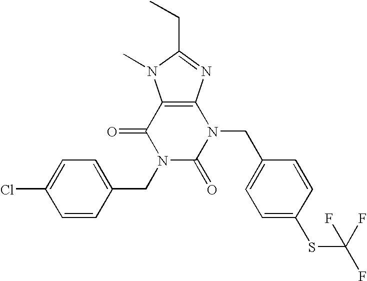

From 31.9 mg (0.1 mmol) 1-(4-chlorobenzyl)-8-ethyl-7-methyl-3,7-dihydro-1H-purine-2,6-dione using potassium carbonate as base, 20.2 mg (0.040 mmol, 40%) of the title compound was isolated.

¹H-NMR (600 MHz, (CH₃)₂SO*, (CD₃)₂SO): δ 7.64 (d, 2H), 7.42 (d, 2H), 7.34-7.25 (m, 4H), 5.19 (s, 2H), 5.01 (s, 2H), 3.82 (s, 3H), 2.73 (q, 2H), 1.19 (t, 3H).

HRMS Calcd for [C₂₃H₂₀ClF₃N₄O₂S+H]⁺: 509.1026. Found: 509.1026.

### Example 26

### 1-(4-chlorobenzyl)-8-ethyl-7-methyl-3-[4-(1H-pyrazol-1-yl)benzyl]-3,7-dihydro-1H-purine-2,6-dione

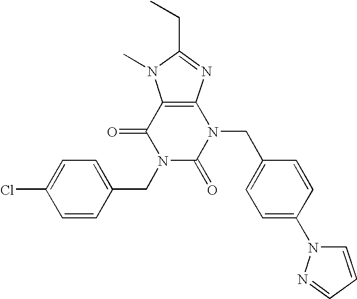

From 31.9 mg (0.1 mmol) 1-(4-chlorobenzyl)-8-ethyl-7-methyl-3,7-dihydro-1H-purine-2,6-dione using potassium carbonate as base, 14.4 mg (0.030 mmol, 30%) of the title compound was isolated.

¹H-NMR (600 MHz, (CH₃)₂SO*, (CD₃)₂SO): δ 8.41 (d, 1H), 7.74 (d, 2H), 7.69 (d, 1H), 7.42 (d, 2H), 7.33 (d, 2H), 7.27 (d, 2H), 6.49 (t, 1H), 5.16 (s, 2H), 5.01 (s, 2H), 3.81 (s, 3H), 2.74 (q, 2H), 1.22 (t, 3H).

HRMS Calcd for [C₂₅H₂₃ClN₆O₂+H]⁺: 475.1649. Found: 475.1648.

### Example 27

### 1-(4-chlorobenzyl)-3-[2-(diethylamino)ethyl]-8-ethyl-7-methyl-3,7-dihydro-1H-purine-2,6-dione

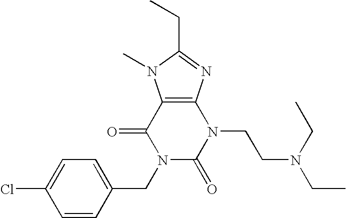

From 31.9 mg (0.1 mmol) 1-(4-chlorobenzyl)-8-ethyl-7-methyl-3,7-dihydro-1H-purine-2,6-dione using potassium carbonate as base, 24.8 mg (0.059 mmol, 59%) of the title compound was isolated.

¹H-NMR (600 MHz, (CH₃)₂SO*, (CD₃)₂SO): δ 7.32 (d, 2H), 7.27 (d, 2H), 5.00 (s, 2H), 4.00 (t, 2H), 3.80 (s, 3H), 2.73 (q, 2H), 2.62 (t, 2H), 2.42 (q, 4H), 1.21 (t, 3H), 0.81 (t, 6H).

HRMS Calcd for [C₂₁H₂₈ClN₅O₂+H]⁺: 418.201. Found: 418.1992.

### Example 28

### 1-(4-chlorobenzyl)-8-ethyl-7-methyl-3-(2-oxo-2-phenylethyl)-3,7-dihydro-1H-purine-2,6-dione

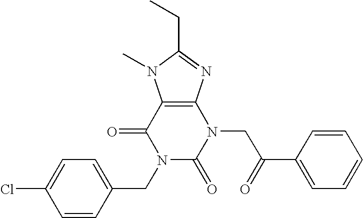

From 31.9 mg (0.1 mmol) 1-(4-chlorobenzyl)-8-ethyl-7-methyl-3,7-dihydro-1H-purine-2,6-dione using potassium carbonate as base, 22.6 mg (0.052 mmol, 52%) of the title compound was isolated.

¹H-NMR (600 MHz, (CH₃)₂SO*, (CD₃)₂SO): δ 8.07 (d, 2H), 7.71 (t, 1H), 7.57 (t, 2H), 7.35 (d, 2H), 7.28 (d, 2H), 5.50 (s, 2H), 5.03 (s, 2H), 3.83 (s, 3H), 2.69 (q, 2H), 1.14 (t, 3H).

HRMS Calcd for [C₂₃H₂₁ClN₄O₃+H]⁺: 437.138. Found: 437.1377.

### Example 29

### 3-(2-{[tert-butyl(dimethyl)silyl]oxy}ethyl)-1-(4-chlorobenzyl)-8-ethyl-7-methyl-3,7-dihydro-1H-purine-2,6-dione

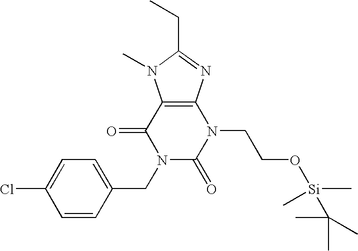

From 31.9 mg (0.1 mmol) 1-(4-chlorobenzyl)-8-ethyl-7-methyl-3,7-dihydro-1H-purine-2,6-dione using potassium carbonate as base, 25.1 mg (0.053 mmol, 53%) of the title compound was isolated.

¹H-NMR (600 MHz, (CH₃)₂SO*, (CD₃)₂SO): [symbol] 7.47 (d, 2H), 7.43 (d, 2H), 5.14 (s, 2H), 4.22 (t, 2H), 3.98-3.94 (m, 5H), 2.87 (q, 2H), 1.35 (t, 3H), 0.83 (s, 9H), 0.00 (s, 6H).

HRMS Calcd for [C₂₃H₃₃ClN₄O₃Si+H]⁺: 477.2089. Found: 477.2082.

### Example 30

### 1-(4-chlorobenzyl)-8-ethyl-7-methyl-3-[(5-methyl-3-phenylisoxazol-4-yl)methyl]-3,7-dihydro-1H-purine-2,6-dione

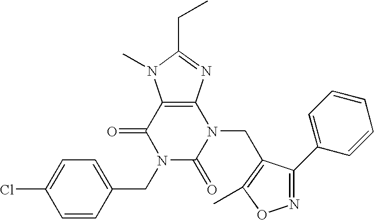

From 31.9 mg (0.1 mmol) 1-(4-chlorobenzyl)-8-ethyl-7-methyl-3,7-dihydro-1H-purine-2,6-dione using potassium carbonate as base, 19.7 mg (0.040 mmol, 40%) of the title compound was isolated.

¹H-NMR (600 MHz, (CH₃)₂SO*, (CD₃)₂SO): δ 7.45 (d, 2H), 7.39 (t, 1H), 7.34-7.30 (m, 4H), 7.18 (d, 2H), 5.06 (s, 2H), 4.91 (s, 2H), 3.73 (s, 3H), 2.65 (q, 2H), 2.33 (s, 3H), 1.14 (t, 3H).

HRMS Calcd for [C₂₆H₂₄ClN₅O₃+H]⁺: 490.1646. Found: 490.1619.

### Example 31

### 1-(4-chlorobenzyl)-8-ethyl-7-methyl-3-[4-(trifluoromethyl)benzyl]-3,7-dihydro-1H-purine-2,6-dione

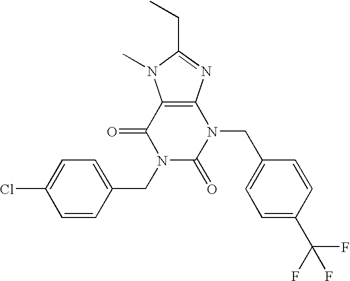

From 31.9 mg (0.1 mmol) 1-(4-chlorobenzyl)-8-ethyl-7-methyl-3,7-dihydro-1H-purine-2,6-dione using potassium carbonate as base, 18.0 mg (0.038 mmol, 38%) of the title compound was isolated.

¹H-NMR (600 MHz, (CH₃)₂SO*, (CD₃)₂SO): [symbol] 7.66 (d, 2H), 7.49 (d, 2H), 7.33 (d, 2H), 7.27 (d, 2H), 5.21 (s, 2H), 5.01 (s, 2H), 3.82 (s, 3H), 2.73 (q, 2H), 1.19 (t, 3H).

HRMS Calcd for [C₂₃H₂₀ClF₃N₄O₂+H]⁺: 477.1305. Found: 477.1299.

### Example 32

### 1-(4-chlorobenzyl)-8-ethyl-3-(2-methoxyethyl)-7-methyl-3,7-dihydro-1H-purine-2,6-dione

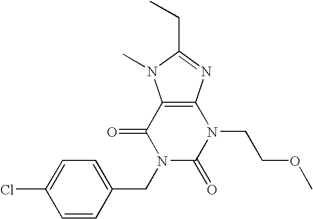

From 31.9 mg (0.1 mmol) 1-(4-chlorobenzyl)-8-ethyl-7-methyl-3,7-dihydro-1H-purine-2,6-dione using potassium carbonate as base, 25.3 mg (0.067 mmol, 67%) of the title compound was isolated.

¹H-NMR (400 MHz, (CD₃)₂SO): [symbol] 7.32 (d, 2H), 7.26 (d, 2H), 4.99 (s, 2H), 4.11 (t, 2H), 3.58 (t, 2H), 3.28 (s, 3H), 3.19 (s, 3H), 2.72 (q, 2H), 1.20 (t, 3H).

HRMS Calcd for [C₁₈H₂₁ClN₄O₃+H]⁺: 377.138. Found: 377.1389.

### Example 33

### 1-(4-chlorobenzyl)-8-ethyl-7-methyl-3-(2-oxobutyl)-3,7-dihydro-1H-purine-2,6-dione

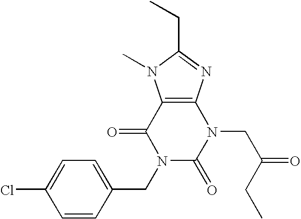

From 31.9 mg (0.1 mmol) 1-(4-chlorobenzyl)-8-ethyl-7-methyl-3,7-dihydro-1H-purine-2,6-dione using potassium carbonate as base, 24.7 mg (0.064 mmol, 64%) of the title compound was isolated.

¹H-NMR (600 MHz, (CH₃)₂SO*, (CD₃)₂SO): [symbol] 7.34 (d, 2H), 7.26 (d, 2H), 4.99 (s, 2H), 4.82 (s, 2H), 3.81 (s, 3H), 2.70 (q, 2H), 2.65-2.43 (m, 2H), 1.16 (t, 3H), 0.94 (t, 3H).

HRMS Calcd for [C₁₉H₂₁ClN₄O₃+H]⁺: 389.138. Found: 389.1371.

### Example 34

### 3-[3-(tert-butylsulfonyl)propyl]-1-(4-chlorobenzyl)-8-ethyl-7-methyl-3,7-dihydro-1H-purine-2,6-dione

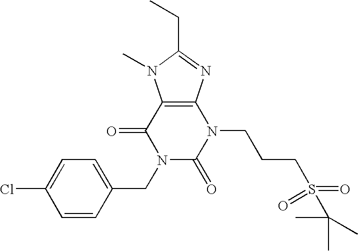

From 31.9 mg (0.1 mmol) 1-(4-chlorobenzyl)-8-ethyl-7-methyl-3,7-dihydro-1H-purine-2,6-dione using potassium carbonate as base, 35.9 mg (0.075 mmol, 75%) of the title compound was isolated.

¹H-NMR (600 MHz, (CH₃)₂SO*, (CD₃)₂SO): [symbol] 7.32 (d, 2H), 7.28 (d, 2H), 5.00 (s, 2H), 4.09 (t, 2H), 3.82 (s, 3H), 3.14-3.09 (m, 2H), 2.74 (q, 2H), 2.09-2.02 (m, 2H), 1.23 (s, 9H), 1.21 (t, 3H).

HRMS Calcd for [C₂₂H₂₉ClN₄O₄S+H]⁺: 481.1676. Found: 481.1662.

### Example 35

### tert-butyl [1-(4-chlorobenzyl)-8-ethyl-7-methyl-2,6-dioxo-1,2,6,7-tetrahydro-3H-purin-3-yl]acetate

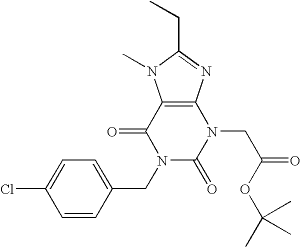

From 31.9 mg (0.1 mmol) 1-(4-chlorobenzyl)-8-ethyl-7-methyl-3,7-dihydro-1H-purine-2,6-dione using potassium carbonate as base, 27.0 mg (0.062 mmol, 62%) of the title compound was isolated.

¹H-NMR (600 MHz, (CH₃)₂SO*, (CD₃)₂SO): [symbol] 7.33 (d, 2H), 7.27 (d, 2H), 5.01 (s, 2H), 4.57 (s, 2H), 3.81 (s, 3H), 2.72 (q, 2H), 1.35 (s, 9H), 1.18 (t, 3H).

HRMS Calcd for [C₂₁H₂₅ClN₄O₄+H]⁺: 433.1642. Found: 433.164.

### Example 36

### 1-(4-chlorobenzyl)-8-ethyl-7-methyl-3-{3-[4-(trifluoromethyl)phenoxy]propyl}-3,7-dihydro-1H-purine-2,6-dione

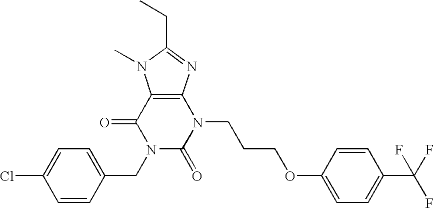

From 31.9 mg (0.1 mmol) 1-(4-chlorobenzyl)-8-ethyl-7-methyl-3,7-dihydro-1H-purine-2,6-dione using potassium carbonate as base, 27.0 mg (0.052 mmol, 52%) of the title compound was isolated.

¹H-NMR (600 MHz, (CH₃)₂SO*, (CD₃)₂SO): [symbol] 7.56 (d, 2H), 7.30 (d, 2H), 7.27 (d, 2H), 6.87 (d, 2H), 4.99 (s, 2H), 4.15 (t, 2H), 4.05 (t, 2H), 3.74 (s, 3H), 2.64-2.55 (m, 2H), 2.15-2.09 (m, 2H), 1.05 (t, 3H).

HRMS Calcd for [C₂₅H₂₄ClF₃N₄O₃+H]⁺: 521.1567. Found: 521.1574.

### Example 37

### 1-(4-chlorobenzyl)-8-ethyl-7-methyl-3-[2-(1H-pyrrol-1-yl)ethyl]-3,7-dihydro-1H-purine-2,6-dione

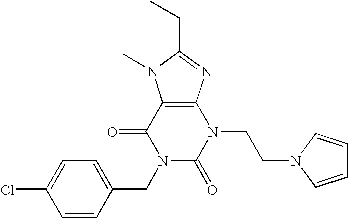

From 31.9 mg (0.1 mmol) 1-(4-chlorobenzyl)-8-ethyl-7-methyl-3,7-dihydro-1H-purine-2,6-dione using potassium carbonate as base, 27.4 mg (0.067 mmol, 67%) of the title compound was isolated.

¹H-NMR (600 MHz, (CH₃)₂SO*, (CD₃)₂SO): [symbol] 7.32 (d, 2H), 7.20 (d, 2H), 6.54 (t, 2H), 5.87 (t, 2H), 4.96 (s, 2H), 4.28-4.19 (m, 4H), 3.78 (s, 3H), 2.71 (q, 2H), 1.20 (t, 3H).

HRMS Calcd for [C₂₁H₂₂ClN₅O₂+H]⁺: 412.154. Found: 412.1543.

### Example 38

### 1-(4-chlorobenzyl)-8-ethyl-3-(3-hydroxypropyl)-7-methyl-3,7-dihydro-1H-purine-2,6-dione

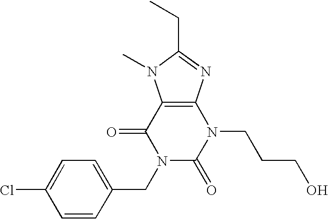

From 31.9 mg (0.1 mmol) 1-(4-chlorobenzyl)-8-ethyl-7-methyl-3,7-dihydro-1H-purine-2,6-dione using potassium carbonate as base, 26.6 mg (0.071 mmol, 71%) of the title compound was isolated.

¹H-NMR (600 MHz, (CH₃)₂SO*, (CD₃)₂SO): [symbol] 7.33 (d, 2H), 7.27 (d, 2H), 5.00 (s, 2H), 4.50 (t, 1H), 4.00 (t, 2H), 3.80 (s, 3H), 3.40 (q, 2H), 2.74 (q, 2H), 1.79-1.74 (m, 2H), 1.21 (t, 3H).

HRMS Calcd for [C₁₈H₂₁ClN₄O₃+H]⁺: 377.138. Found: 377.1407.

### Example 39

### 1-(4-chlorobenzyl)-3-{[3-chloro-4-(isopropylsulfonyl)-2-thienyl]methyl}-8-ethyl-7-methyl-3,7-dihydro-1H-purine-2,6-dione

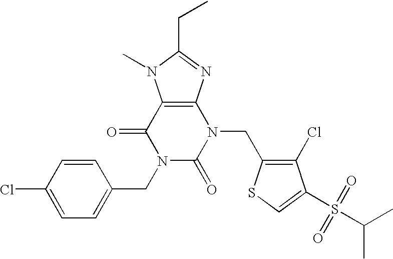

From 31.9 mg (0.1 mmol) 1-(4-chlorobenzyl)-8-ethyl-7-methyl-3,7-dihydro-1H-purine-2,6-dione using potassium carbonate as base, 42.3 mg (0.076 mmol, 76%) of the title compound was isolated.

¹H-NMR (600 MHz, (CH₃)₂SO*, (CD₃)₂SO): [symbol] 8.37 (s, 1H), 7.33 (d, 2H), 7.27 (d, 2H), 5.33 (s, 2H), 5.02 (s, 2H), 3.80 (s, 3H), 3.48-3.43 (m, 1H), 2.74 (q, 2H), 1.23 (t, 3H), 1.16 (d, 6H).

HRMS Calcd for [C₂₃H₂₄Cl₂N₄O₄S₂+H]⁺: 555.0694. Found: 555.0686.

### Example 40

### 1-(3,4-Dichlorobenzyl)-3-(3,3-dimethyl-2-oxobutyl)-8-ethyl-7-methyl-3,7-dihydro-1H-purine-2,6-dione

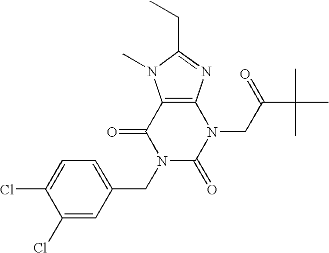

From 76 mg (0.238 mmol) 1-(3,4-dichlorobenzyl)-8-ethyl-7-methyl-3,7-dihydro-1H-purine-2,6-dione using potassium carbonate as base, 68 mg (0.149 mmol, 68%) of the title compound was isolated.

¹H-NMR (400 MHz, CDCl₃) δ 7.51-7.46 (m, 1H), 7.35-7.29 (m, 1H), 7.27-7.21 (m, 1H), 5.09 (s, 2H), 5.01 (s, 2H), 3.84 (s, 3H), 2.65 (q, 2H), 1.30-1.20 (m, 12H)

MS m/z 451.1290 (M+H)⁺

### Example 41

### 1-(3,4-Dichlorobenzyl)-3-(3,3-dimethylbutyl)-8-ethyl-7-methyl-3,7-dihydro-1H-purine-2,6-dione

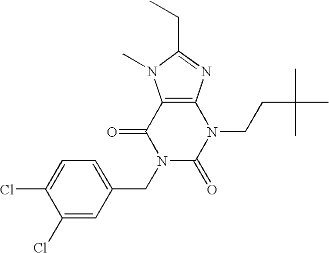

From 76 mg (0.238 mmol) 1-(3,4-dichlorobenzyl)-8-ethyl-7-methyl-3,7-dihydro-1H-purine-2,6-dione using potassium carbonate as base, 36 mg (0.082 mmol, 33%) of the title compound was isolated.

¹H-NMR (400 MHz, CDCl₃) δ 7.53-7.47 (m, 1H), 7.35-7.25 (m, 2H), 5.08 (s, 2H), 4.16-4.00 (m, 2H), 3.87 (s, 3H), 2.71 (q, 2H), 1.67-1.54 (m, 2H), 1.32 (t, 3H), 0.98 (s, 9H)

MS m/z 437.1513 (M+H)⁺

### Example 42

### 3-{2-[(3S,5S,7S)-Adamantan-1-yl]-2-oxoethyl}-1-(3,4-dichlorobenzyl)-8-ethyl-7-methyl-3,7-dihydro-1H-purine-2,6-dione

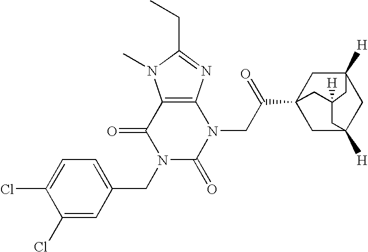

From 50 mg (0.142 mmol) 1-(3,4-dichlorobenzyl)-8-ethyl-7-methyl-3,7-dihydro-1H-purine-2,6-dione using potassium carbonate as base, 41 mg (0.075 mmol, 53%) of the title compound was isolated.

¹H-NMR (400 MHz, CDCl₃) δ 7.50-7.46 (m, 1H), 7.34-7.29 (m, 1H), 7.26-7.20 (m, 1H), 5.08 (s, 2H), 4.98 (s, 2H), 3.84 (s, 3H), 2.65 (q, 2H), 2.11-2.01 (m, 3H), 1.99-1.90 (m, 6H), 1.80-1.66 (m, 6H), 1.26 (t, 3H)

MS m/z 529.1779 (M+H)⁺

### Example 43

### 1-(3,4-Dichlorobenzyl)-8-ethyl-7-methyl-3-[2-(trimethylsilyl)ethyl]-3,7-dihydro-1H-purine-2,6-dione

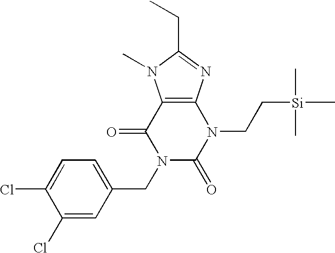

From 38 mg (0.108 mmol) 1-(3,4-dichlorobenzyl)-8-ethyl-7-methyl-3,7-dihydro-1H-purine-2,6-dione using potassium carbonate as base, 21 mg (0.044 mmol, 40%) of the title compound was isolated.

¹H-NMR (600 MHz, (CH₃)₂SO*, (CD₃)₂SO)) δ 7.57-7.56 (m, 1H), 7.56-7.55 (m, 1H), 7.52-7.51 (m, 1H), 5.02 (s, 2H), 4.06-4.00 (m, 2H), 3.82 (s, 3H), 2.76 (q, 2H), 1.2 (t, 3H), 1.02-0.95 (m, 2H), 0.00 (s, 9H)

MS m/z 453.1285 (M+H)⁺

### Example 44

### 3-(4-Cyclohexylbutyl)-1-(3,4-dichlorobenzyl)-8-ethyl-7-methyl-3,7-dihydro-1H-purine-2,6-dione

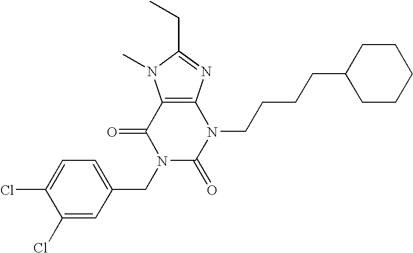

From 40 mg (0.113 mmol) 1-(3,4-dichlorobenzyl)-8-ethyl-7-methyl-3,7-dihydro-1H-purine-2,6-dione using potassium carbonate as base, 21 mg (0.042 mmol, 37%) of the title compound was isolated.

¹H-NMR (600 MHz, (CH₃)₂SO*, (CD₃)₂SO)) δ 7.54-7.52 (m, 1H), 7.50-7.48 (m, 1H), 7.24-7.21 (m, 1H), 5.00 (s, 2H), 3.94 (t, 2H), 3.81 (s, 3H), 2.73 (q, 2H), 1.63-1.52 (m, 5H), 1.20 (t, 3H), 1.16-1.00 (m, 10H), 0.80-0.71 (m, 2H)

MS m/z 491.1977 (M+H)⁺

### Example 45

### 8-tert-butyl-1-(4-chlorobenzyl)-7-methyl-3-propyl-3,7-dihydro-1H-purine-2,6-dione

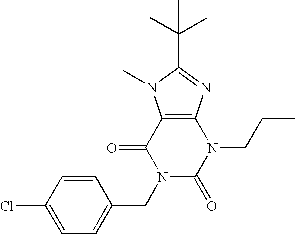

From 50 mg (0.162 mmol) 5,6-diamino-3-(4-chlorobenzyl)-1-propylpyrimidine-2,4(1H,3H)-dione, 20 mg (0.052 mmol, 33%) of the title compound were isolated.

¹H-NMR (600 MHz, (CH₃)₂SO*, (CD₃)₂SO)) δ 7.32-7.34 (m, 2H), 7.25-7.28 (m, 2H), 5.00 (s, 2H), 4.02 (s, 3H), 3.89-3.92 (m, 2H), 1.62-1.68 (m, 2H), 1.38 (s, 9H), 0.83 (t, 7.3 Hz, 3H)

HRMS Calcd for [C₂₀H₂₅ClN₄O₂+H]⁺: 389.1744. Found: 389.1748.

### Example 46

### Synthesis of 1-(4-chlorobenzyl)-8-ethyl-7-methyl-3-(3-pyrrolidin-1-ylpropyl)-3,7-dihydro-1H-purine-2,6-dione

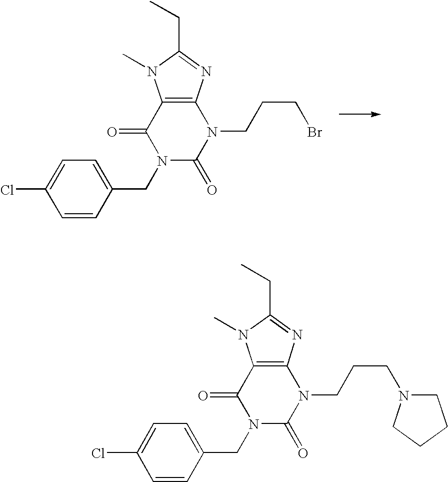

3-(3-Bromopropyl)-1-(4-chlorobenzyl)-8-ethyl-7-methyl-3,7-dihydro-1H-purine-2,6-dione (50 mg, 0.114 mmol) was dissolved in DMF (1.3 mL) and then K₂CO₃ (47 mg, 0.342 mmol) and pyrrolidine (16 mg, 0.227 mmol) was added. The reaction mixture was allowed to stir at rt overnight. Water was added and a precipitate occurred. The solid was collected, dissolved in DMSO (1.2 mL) and purified by HPLC to yield 14 mg (0.030 mmol, 27%) of the title compound.

¹H-NMR (600 MHz, (CH₃)₂SO*, (CD₃)₂SO)) δ 7.36-7.30 (m, 2H), 7.29-7.21 (m, 2H), 4.99 (s, 2H), 4.00 (t, 2H), 3.79 (s, 3H), 2.72 (q, 2H), 2.41-2.29 (m, 6H), 1.80-1.73 (m, 2H), 1.64-1.52 (m, 4H), 1.20 (t, 3H)

MS m/z 430.2001 (M+H)⁺

The following compounds were synthesized according to example 46:

### Example 47

### 1-(4-Chlorobenzyl)-8-ethyl-7-methyl-3-[3-(4-phenylpiperazin-1-yl)propyl]-3,7-dihydro-1H-purine-2,6-dione

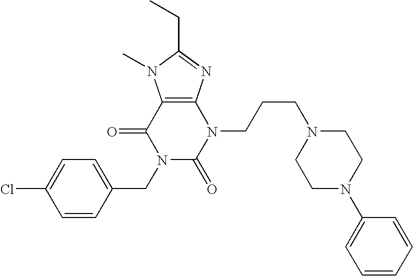

From 40 mg (0.091 mmol) 3-(3-bromopropyl)-1-(4-chlorobenzyl)-8-ethyl-7-methyl-3,7-dihydro-1H-purine-2,6-dione, 21 mg (0.038 mmol, 41%) of the title compound was isolated.

¹H-NMR (600 MHz, (CH₃)₂SO*, (CD₃)₂SO)) δ 7.34-7.30 (m, 2H), 7.30-7.26 (m, 2H), 7.18-7.13 (m, 2H), 6.86-6.81 (m, 2H), 6.75-6.71 (m, 1H), 4.99 (s, 2H), 4.03 (t, 2H), 3.79 (s, 3H), 2.95-2.90 (m, 4H), 2.73 (q, 2H), 2.40-2.30 (m, 6H), 1.88-1.79 (m, 2H), 1.21 (t, 3H)

MS m/z 521.2416 (M+H)⁺

### Example 48

### 1-(4-Chlorobenzyl)-3-[3-(1,4-dioxa-8-azaspiro[4.5]dec-8-yl)propyl]-8-ethyl-7-methyl-3,7-dihydro-1H-purine-2,6-dione

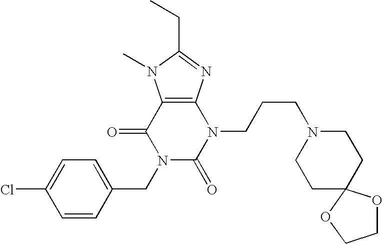

From 40 mg (0.091 mmol) 3-(3-bromopropyl)-1-(4-chlorobenzyl)-8-ethyl-7-methyl-3,7-dihydro-1H-purine-2,6-dione, 21 mg (0.038 mmol, 42%) of the title compound was isolated.

¹H-NMR (400 MHz, (CDCl₃) δ 7.43-7.35 (m, 2H), 7.24-7.17 (m, 2H) 5.10 (s, 2H), 4.10 (t, 2H), 3.90 (s, 4H), 3.86 (s, 3H), 2.69 (q, 2H), 2.49-2.36 (m, 6H), 1.96-1.84 (m, 2H), 1.65 (t, 4H), 1.28 (t, 3H)

MS m/z 502.2224 (M+H)⁺

### Example 49

### 1-(4-Chlorobenzyl)-8-ethyl-3-[3-(1H-imidazol-1-yl)propyl]-7-methyl-3,7-dihydro-1H-purine-2,6-dione

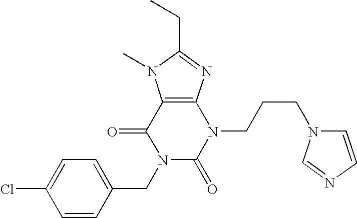

From 40 mg (0.091 mmol) 3-(3-bromopropyl)-1-(4-chlorobenzyl)-8-ethyl-7-methyl-3,7-dihydro-1H-purine-2,6-dione, 16 mg (0.036 mmol, 40%) of the title compound was isolated.

¹H-NMR (600 MHz, (CH₃)₂SO*, (CD₃)₂SO)) δ 7.60 (s, 1H), 7.35-7.30 (m, 2H), 7.30-7.25 (m, 2H), 7.15 (s, 1H), 6.82 (s, 1H), 4.99 (s, 2H), 3.99-3.92 (m, 4H), 3.80 (s, 3H), 2.73 (q, 2H), 2.11-2.05 (m, 2H), 1.80-1.73 (m, 2H), 1.21 (t, 3H)

MS m/z 427.1634 (M+H)⁺

### Example 50

### Synthesis of 1-(4-Chlorobenzyl)-3-[2-(3,3-difluoropyrrolidin-1-yl)-2-oxoethyl]-8-ethyl-7-methyl-3,7-dihydro-1H-purine-2,6-dione

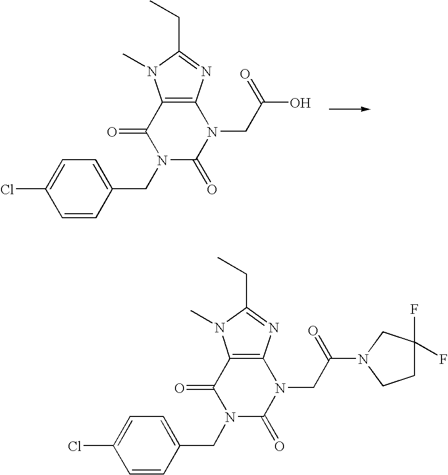

[1-(4-Chlorobenzyl)-8-ethyl-7-methyl-2,6-dioxo-1,2,6,7-tetrahydro-3H-purin-3-yl]acetic acid (99 mg, 0.263 mmol) was dissolved in dichloromethane (1 mL), then 3,3-difluoropyrrolidine hydrochloride (58 mg, 0.404 mmol), TBTU (93 mg, 0.289 mmol), and DIPEA (0.19 mL, 1.05 mmol) were added. The reaction mixture was stirred at rt for 1 h. Water was added, the organic layer was separated and dried by filtration through a phase separator. The solvents were evaporated and the residue dissolved in DMSO and purified by HPLC. 47 mg (0.10 mmol, 38%) of the title compound was isolated.

¹H-NMR (500 MHz, CDCl₃) δ 7.43-7.37 (m, 2H), 7.30-7.24 (m, 2H), 5.15 (s, 2H), 4.83 (s, 1H) 4.75 (s, 1H), 4.01-3.82 (m, 6H), 3.78 (t, 1H), 2.74 (q, 2H), 2.58-2.46 (m, 1H), 2.44-2.32 (m, 1H), 1.31 (t, 3H)

MS m/z 466.1451 (M+H)⁺

The following compound was synthesized according to example 50:

### Example 51

### 2-[1-(4-Chlorobenzyl)-8-ethyl-7-methyl-2,6-dioxo-1,2,6,7-tetrahydro-3H-purin-3-yl]-N,N-diisopropylacetamide

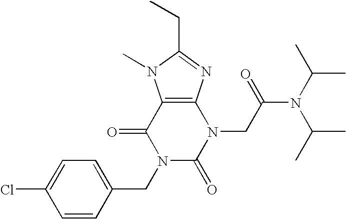

From 106 mg (0.281 mmol) [1-(4-Chlorobenzyl)-8-ethyl-7-methyl-2,6-dioxo-1,2,6,7-tetrahydro-3H-purin-3-yl]acetic acid, 30 mg (0.065 mmol, 23%) of the title compound was isolated.

¹H-NMR (500 MHz, CDCl₃) δ 7.43-7.36 (m, 2H), 7.30-7.22 (m, 2H), 5.17 (s, 2H), 4.85 (s, 2H) 4.05-3.93 (m, 1H), 3.86 (s, 3H), 3.61-3.47 (br, 1H), 2.76 (q, 2H), 1.43-1.23 (m, 15H)

MS m/z 460.2106 (M+H)⁺

### Example 52

### Synthesis of 1-(4-chlorobenzyl)-3-(2,2-dimethoxyethyl)-8-ethyl-7-methyl-3,7-dihydro-1H-purine-2,6-dione

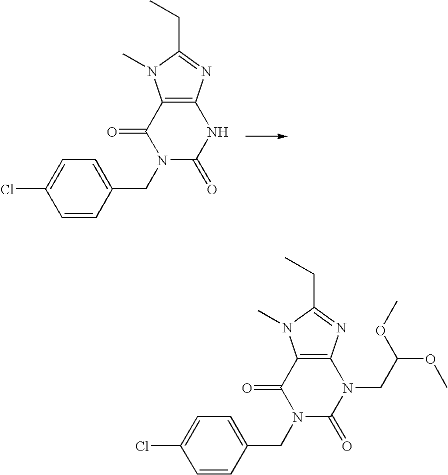

1-(4-Chlorobenzyl)-8-ethyl-7-methyl-3,7-dihydro-1H-purine-2,6-dione (206 mg, 0.646 mmol) was dissolved in DMSO (5 mL) and finely powdered KOH (60 mg, 1.07 mmol) was added. The reaction mixture was stirred at rt for 10 minutes, then 2-bromo-1,1-dimethoxymethane (0.437 mg, 2.59 mmol) was added. The reaction mixture was stirred for 2 h at rt, then transferred to a vial, sealed and heated for 80 min to 120° C. using microwave heating. Then water was added to the reaction and extracted with EtOAc twice. The combined organic layers were washed three times with water. The organic layer was dried by filtration through a phase separator and evaporated. (140 mg, 0.307 mmol, 47%) of the crude title compound was isolated.

¹H-NMR (500 MHz, CDCl₃) δ 7.46-7.39 (m, 2H), 7.30-7.24 (m, 2H), 5.16 (s, 2H), 4.96 (t, 1H) 4.23 (d, 2H), 3.90 (s, 3H), 3.39 (s, 6H), 2.75 (q, 2H), 1.34 (t, 3H)

MS m/z 407.1487 (M+H)⁺

### Example 53

### Synthesis of 1-(4-Chlorobenzyl)-3-(2,3-dihydroxypropyl)-8-ethyl-7-methyl-3,7-dihydro-1H-purine-2,6-dione

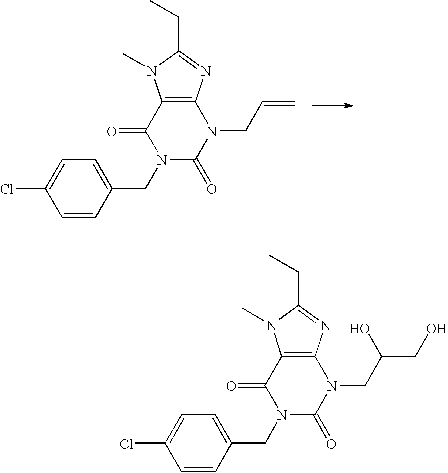

3-Allyl-1-(4-chlorobenzyl)-8-ethyl-7-methyl-3,7-dihydro-1H-purine-2,6-dione (273 mg, 0.746 mmol) was dissolved in acetone (10 mL) and 4-methylmorpholine-4-oxide (205 mg, 1.75 mmol) was added and the reaction mixture was stirred until everything was dissolved, 2.5% osmium tetroxide (0.191 mL, 0.015 mmol) was added. The reaction mixture was stirred at rt for 2 h. The reaction was quenched with 39% NaHSO₃ (aq, 15 mL), which was stirred at rt for 30 min. Water was added and extracted with dichloromethane (twice). The combined organic layer was dried by filtration through a phase separator and evaporated to yield the crude title compound (280 mg, 0.627 mmol, 82%).

¹H-NMR (400 MHz, CDCl₃) δ 7.40-7.31 (m, 2H), 7.22-7.14 (m, 2H), 5.08 (s, 2H), 4.33-4.19 (m, 2H), 4.02-3.94 (m, 1H), 3.85 (s, 3H), 3.69-3.64 (m, 1H), 3.53-3.37 (m, 1H), 2.69 (q, 2H), 1.29 (t, 3H)

MS m/z 393.2 (M+H)⁺

### Example 54

### Synthesis of 1-(4-chlorobenzyl)-8-ethyl-7-methyl-3-propyl-3,7-dihydro-1H-purine-2,6-dione

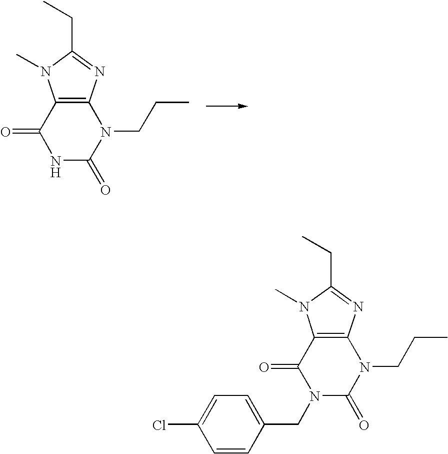

75 mg (0.317 mmol) 8-ethyl-7-methyl-3-propyl-3,7-dihydro-1H-purine-2,6-dione was dissolved in 2 mL DMF, then 207 mg (0.635 mmol) Cs₂CO₃, 4 mg (0.01 mmol) tetrabutylammonium iodide and 78 mg (0.38 mmol) 4-chlorobenzyl bromide was added. After stirring at rt for 1 h, water was added and extracted 3 times with ethyl acetate. The combined organic layers were dried by filtration through a phase separator and evaporated. The residue was purified by HPLC and flash chromatography. 81 mg (0.224 mmol, 70%) of the title compound was isolated.

¹H-NMR (400 MHz, CDCl₃) δ 7.46-7.41 (m, 2H), 7.30-7.24 (m, 2H), 5.16 (s, 2H), 4.05 (t, 2H), 3.92 (s, 3H), 2.76 (q, 2H), 1.84-1.74 (m, 2H), 1.34 (t, 3H), 0.97 (t, 3H)

MS m/z 361.1444 (M+H)⁺

The following compounds were synthesized according to example 54:

### Example 55

### Methyl 4-[(8-Ethyl-7-methyl-2,6-dioxo-3-propyl-2,3,6,7-tetrahydro-1H-purin-1-yl)methyl]benzoate

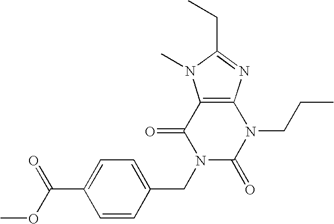

From 59 mg (0.250 mmol) 8-ethyl-7-methyl-3-propyl-3,7-dihydro-1H-purine-2,6-dione, 3 mg (0.008 mmol, 3%) of the title compound was isolated.

¹H-NMR (600 MHz, (CH₃)₂SO*, (CD₃)₂SO)) δ 7.87-7.82 (m, 2H), 7.36-7.30 (m, 2H), 5.08 (s, 2H), 3.90 (t, 2H), 3.82-3.77 (m, 6H), 2.73 (q, 2H), 1.68-1.60 (m, 2H), 1.20 (t, 3H), 0.82 (t, 3H)

MS m/z 385.1887 (M+H)⁺

### Example 56

### 1-[4-(Benzyloxy)benzyl]-8-ethyl-7-methyl-3-propyl-3,7-dihydro-1H-purine-2,6-dione

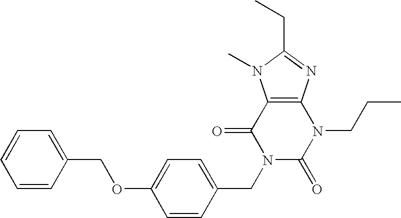

From 76 mg (0.320 mmol) 8-ethyl-7-methyl-3-propyl-3,7-dihydro-1H-purine-2,6-dione, 50 mg (0.116 mmol, 36%) of the title compound was isolated.

¹H-NMR (400 MHz, CDCl₃) δ 7.46-7.22 (m, 7H), 6.91-6.83 (m, 2H), 5.11 (s, 2H), 5.00 (s, 2H), 4.05-3.97 (m, 2H), 3.87 (s, 3H), 2.72 (q, 2H), 1.81-1.65 (m, 2H), 1.29 (t, 3H), 0.93 (t, 3H)

MS m/z 433.2238 (M+H)⁺

### Example 57

### 1-(3,4-Dichlorobenzyl)-8-ethyl-7-methyl-3-propyl-3,7-dihydro-1H-purine-2,6-dione

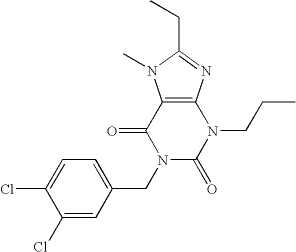

From 30 mg (0.127 mmol) 8-ethyl-7-methyl-3-propyl-3,7-dihydro-1H-purine-2,6-dione, 29 mg (0.073 mmol, 57%) of the title compound was isolated.

¹H-NMR (600 MHz, (CH₃)₂SO*, (CD₃)₂SO)) δ 7.54-7.47 (m, 2H), 7.23-7.20 (m, 1H), 5.00 (s, 2H), 3.90 (t, 2H), 3.79 (s, 3H), 2.73 (q, 2H), 1.68-1.59 (m, 2H), 1.22-1.17 (m, 3H), 0.82 (t, 3H)

MS m/z 395.1042 (M+H)⁺

### Example 58

### 8-Ethyl-7-methyl-1-(2-naphthylmethyl)-3-propyl-3,7-dihydro-1H-purine-2,6-dione

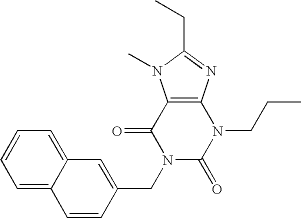

From 38 mg (0.161 mmol) 8-ethyl-7-methyl-3-propyl-3,7-dihydro-1H-purine-2,6-dione using potassium carbonate as base, 19 mg (0.050 mmol, 31%) of the title compound was isolated.

¹H-NMR (600 MHz, (CH₃)₂SO*, (CD₃)₂SO)) δ 7.86-7.79 (m, 3H), 7.70 (s, 1H), 7.47-7.40 (m, 3H), 5.19 (s, 2H), 3.94-3.90 (m, 2H), 3.81 (s, 3H), 2.73 (q, 2H), 1.69-1.60 (m, 2H), 1.20 (t, 3H), 0.83 (t, 3H)

MS m/z 377.1973 (M+H)⁺

### Example 59

### 1-{[1-(4-Chlorophenyl)-5-(trifluoromethyl)-1H-pyrazol-4-yl]methyl}-8-ethyl-7-methyl-3-propyl-3,7-dihydro-1H-purine-2,6-dione

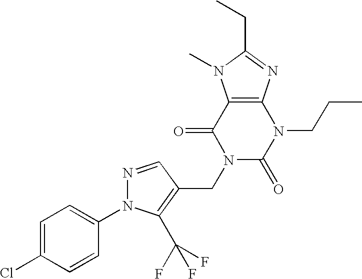

From 30 mg (0.127 mmol) 8-ethyl-7-methyl-3-propyl-3,7-dihydro-1H-purine-2,6-dione, 24 mg (0.048 mmol, 37%) of the title compound was isolated.

¹H-NMR (600 MHz, (CH₃)₂SO*, (CD₃)₂SO)) δ 7.62-7.56 (m, 3H), 7.50-7.46 (m, 2H), 5.08 (s, 2H), 3.93 (t, 2H), 3.81 (s, 3H), 2.74 (q, 2H), 1.70-1.61 (m, 2H), 1.21 (t, 3H), 0.84 (t, 3H)

MS m/z 495.1545 (M+H)⁺

### Example 60

### 1-(2,4-Dichlorobenzyl)-8-ethyl-7-methyl-3-propyl-3,7-dihydro-1H-purine-2,6-dione

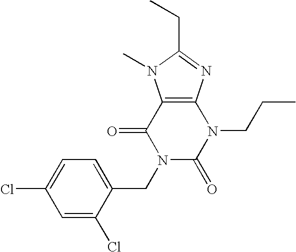

From 24 mg (0.10 mmol) 8-ethyl-7-methyl-3-propyl-3,7-dihydro-1H-purine-2,6-dione using potassium carbonate as base, 9 mg (0.020 mmol, 20%) of the title compound was isolated.

¹H-NMR (400 MHz, CDCl₃) δ 7.37-7.32 (m, 1H), 7.12-7.05 (m, 1H), 6.91-6.85 (m, 1H), 5.23 (s, 2H), 4.04 (t, 2H), 3.87 (s, 3H), 2.74 (q, 2H), 1.83-1.68 (m, 2H), 1.32 (t, 3H), 0.92 (t, 3H)

MS m/z 395.2052 (M+H)⁺

### Example 61

### 1-(4-Bromobenzyl)-8-ethyl-7-methyl-3-propyl-3,7-dihydro-1H-purine-2,6-dione

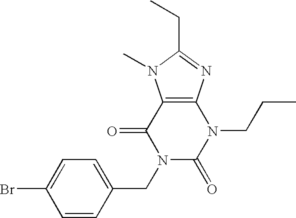

From 38 mg (0.16 mmol) 8-ethyl-7-methyl-3-propyl-3,7-dihydro-1H-purine-2,6-dione using potassium carbonate as base, 37 mg (0.090 mmol, 56%) of the title compound was isolated.

¹H-NMR (600 MHz, (CH₃)₂SO*, (CD₃)₂SO)) δ 7.48-7.44 (m, 2H), 7.22-7.18 (m, 2H), 4.98 (s, 2H), 3.90 (t, 2H), 3.80 (s, 3H), 2.73 (q, 2H), 1.68-1.59 (m, 2H), 1.20 (t, 3H), 0.83 (t, 3H)

MS m/z) 405.0942 (M)⁺

### Example 62

### 8-Ethyl-7-methyl-3-propyl-1-[4-(trifluoromethyl)benzyl]-3,7-dihydro-1H-purine-2,6-dione

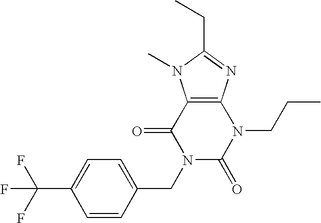

From 59 mg (0.25 mmol) 8-ethyl-7-methyl-3-propyl-3,7-dihydro-1H-purine-2,6-dione using cesium carbonate as base, 38.4 mg (0.097 mmol, 38.9%) of the title compound was isolated.

¹H-NMR (600 MHz, (CH₃)₂SO*, (CD₃)₂SO)) δ 7.66-7.61 (m, 2H), 7.22-7.18 (m, 2H), 5.09 (s, 2H), 3.91 (t, 2H), 3.80 (s, 3H), 2.73 (q, 2H), 1.68-1.60 (m, 2H), 1.20 (t, 3H), 0.83 (t, 3H)

MS m/z 395.1710 (M+H)⁺

### Example 63

### 1-[2-(4-Chlorophenyl)ethyl]-8-ethyl-7-methyl-3-propyl-3,7-dihydro-1H-purine-2,6-dione

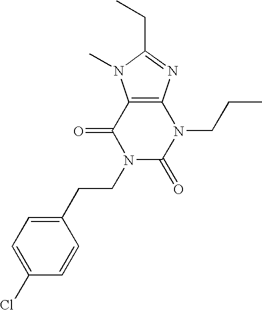

From 59 mg (0.25 mmol) 8-ethyl-7-methyl-3-propyl-3,7-dihydro-1H-purine-2,6-dione using cesium carbonate as base, 22.9 mg (0.061 mmol, 24.4%) of the title compound was isolated.

¹H-NMR (600 MHz, (CH₃)₂SO*, (CD₃)₂SO)) δ 7.33-7.28 (m, 2H), 7.20-7.17 (m, 2H), 4.03 (t, 2H), 3.87 (t, 2H), 3.79 (s, 3H), 2.94 (t, 2H), 2.72 (q, 2H), 1.64-1.55 (m, 2H), 1.19 (t, 3H), 0.80 (t, 3H)

MS m/z 361.1424 (M+H)⁺

### Example 64

### 1-(2,1,3-Benzothiadiazol-5-ylmethyl)-8-ethyl-7-methyl-3-propyl-3,7-dihydro-1H-purine-2,6-dione

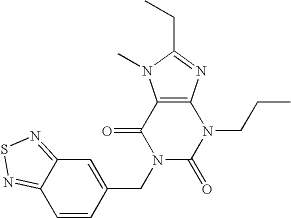

From 59 mg (0.25 mmol) 8-ethyl-7-methyl-3-propyl-3,7-dihydro-1H-purine-2,6-dione using cesium carbonate as base, 13.9 mg (0.036 mmol, 14.5%) of the title compound was isolated.

¹H-NMR (600 MHz, (CH₃)₂SO*, (CD₃)₂SO)) δ 8.04-8.00 (m, 1H), 7.80 (s, 1H), 7.67-7.64 (m, 1H), 5.23 (s, 2H), 3.92 (t, 2H), 3.81 (s, 3H), 2.74 (q, 2H), 1.69-1.61 (m, 2H), 1.21 (t, 3H), 0.83 (t, 3H)

MS m/z 385.1456 (M+H)⁺

### Example 65

### 4-[(8-Ethyl-7-methyl-2,6-dioxo-3-propyl-2,3,6,7-tetrahydro-1H-purin-1-yl)methyl]benzonitrile

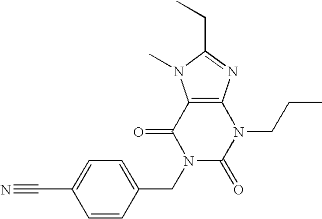

From 59 mg (0.25 mmol) 8-ethyl-7-methyl-3-propyl-3,7-dihydro-1H-purine-2,6-dione using cesium carbonate as base, 18.3 mg (0.052 mmol, 20.8%) of the title compound was isolated.

¹H-NMR (600 MHz, (CH₃)₂SO*, (CD₃)₂SO)) δ 7.75-7.71 (m, 2H), 7.42-7.37 (m, 2H), 5.09 (s, 2H), 3.92 (t, 2H), 3.81 (s, 3H), 2.74 (q, 2H), 1.69-1.61 (m, 2H), 1.21 (t, 3H), 0.83 (t, 3H)

MS m/z 352.1785 (M+H)⁺

### Example 66

### 8-Ethyl-7-methyl-3-propyl-1-{[5-(trifluoromethyl)-1,3-benzothiazol-2-yl]methyl}-3,7-dihydro-1H-purine-2,6-dione

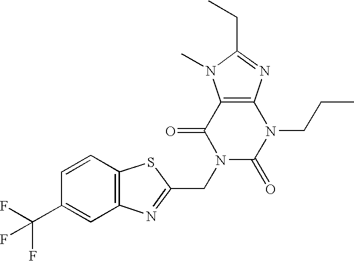

From 59 mg (0.25 mmol) 8-ethyl-7-methyl-3-propyl-3,7-dihydro-1H-purine-2,6-dione using cesium carbonate as base, 10 mg (0.022 mmol, 8.9%) of the title compound was isolated.

¹H-NMR (600 MHz, (CH₃)₂SO*, (CD₃)₂SO)) δ 8.30-8.26 (m, 2H), 7.74-7.71 (m, 1H), 5.49 (s, 2H), 3.94 (t, 2H), 3.81 (s, 3H), 2.76 (q, 2H), 1.70-1.62 (m, 2H), 1.22 (t, 3H), 0.84 (t, 3H)

MS m/z 452.1381 (M+H)⁺

### Example 67

### 1-(3-Chlorobenzyl)-8-ethyl-7-methyl-3-propyl-3,7-dihydro-1H-purine-2,6-dione

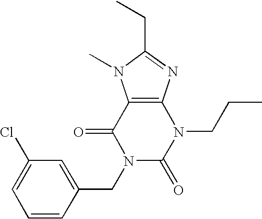

From 59 mg (0.25 mmol) 8-ethyl-7-methyl-3-propyl-3,7-dihydro-1H-purine-2,6-dione using cesium carbonate as base, 11.2 mg (0.031 mmol, 12.4%) of the title compound was isolated.

¹H-NMR (600 MHz, (CH₃)₂SO*, (CD₃)₂SO)) δ 7.32-7.18 (m, 3H), 4.99 (s, 2H), 3.91 (t, 2H), 3.80 (s, 3H), 2.73 (q, 2H), 1.68-1.60 (m, 2H), 1.20 (t, 3H), 0.82 (t, 3H)

MS m/z 361.1432 (M+H)⁺

### Example 68

### 1-(4-Benzoylbenzyl)-8-ethyl-7-methyl-3-propyl-3,7-dihydro-1H-purine-2,6-dione

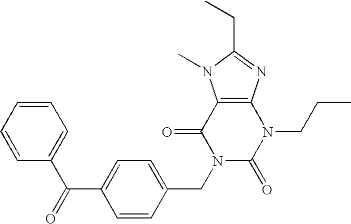

From 59 mg (0.25 mmol) 8-ethyl-7-methyl-3-propyl-3,7-dihydro-1H-purine-2,6-dione using cesium carbonate as base, 24.2 mg (0.056 mmol, 22.5%) of the title compound was isolated.

¹H-NMR (600 MHz, (CH₃)₂SO*, (CD₃)₂SO)) δ 7.70-7.61 (m, 5H), 7.54-7.49 (m, 2H), 7.41-7.37 (m, 2H), 5.12 (s, 2H), 3.92 (t, 2H), 3.81 (s, 3H), 2.74 (q, 2H), 1.69-1.61 (m, 2H), 1.21 (t, 3H), 0.83 (t, 3H)

MS m/z 431.2081 (M+H)⁺

### Example 69

### 8-Ethyl-1-(4-methoxybenzyl)-7-methyl-3-propyl-3,7-dihydro-1H-purine-2,6-dione

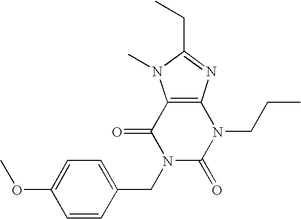

From 59 mg (0.25 mmol) 8-ethyl-7-methyl-3-propyl-3,7-dihydro-1H-purine-2,6-dione using cesium carbonate as base, 10.1 mg (0.028 mmol, 11.3%) of the title compound was isolated.

¹H-NMR (600 MHz, (CH₃)₂SO*, (CD₃)₂SO)) δ 7.22-7.18 (m, 2H), 6.98-6.79 (m, 2H), 4.95 (s, 2H), 3.89 (t, 2H), 3.80 (s, 3H), 3.67 (s, 3H), 2.72 (q, 2H), 1.67-1.59 (m, 2H), 1.19 (t, 3H), 0.82 (t, 3H)

MS m/z 357.1955 (M+H)⁺

### Example 70

### 8-Ethyl-1-(4-isopropylbenzyl)-7-methyl-3-propyl-3,7-dihydro-1H-purine-2,6-dione

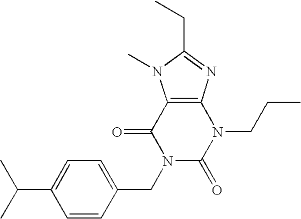

From 59 mg (0.25 mmol) 8-ethyl-7-methyl-3-propyl-3,7-dihydro-1H-purine-2,6-dione using cesium carbonate as base, 25.9 mg (0.070 mmol, 28.1%) of the title compound was isolated.

¹H-NMR (600 MHz, (CH₃)₂SO*, (CD₃)₂SO)) δ 7.17-7.10 (m, 4H), 4.98 (s, 2H), 3.90 (t, 2H), 3.80 (s, 3H), 2.82-2.77 (m, 1H), 2.72 (q, 2H), 1.68-1.60 (m, 2H), 1.20 (t, 3H), 1.13 (d, 6H, 0.83 (t, 3H)

MS m/z 369.2296 (M+H)⁺

### Example 71

### 1-(4-Chlorobenzyl)-3-(2,4-dimethoxybenzyl)-8-ethyl-7-methyl-3,7-dihydro-1H-purine-2,6-dione

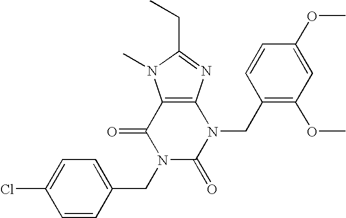

From 1 g (2.90 mmol) 3-(2,4-dimethoxybenzyl)-8-ethyl-7-methyl-3,7-dihydro-1H-purine-2,6-dione, 1.4 g (2.98 mmol, 102%) crude title compound was isolated. A sample was purified by reversed phase HPLC.

¹H-NMR (400 MHz, CDCl₃) δ 7.32-7.25 (m, 2H), 7.15-7.08 (m, 2H), 6.82-6.77 (m, 1H), 6.32-6.28 (m, 1H), 6.25-6.20 (m, 1H), 5.02 (s, 2H), 3.77 (s, 3H), 3.67-3.61 (m, 6H), 2.59 (q, 2H), 1.17 (t, 3H)

MS m/z 469.1625 (M+H)⁺

### Example 72

### 1-(4-Chlorobenzyl)-7,8-diethyl-3-propyl-3,7-dihydro-H-purine-2,6-dione

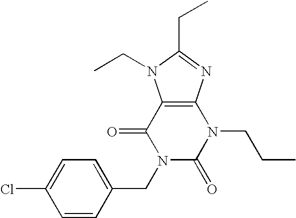

From 196 mg (0.78 mmol) 7,8-diethyl-3-propyl-3,7-dihydro-1H-purine-2,6-dione using potassium carbonate as a base, 76 mg (0.203 mmol, 26%) of the title compound was isolated.

¹H NMR (400 MHz, CDCl₃) δ 7.40 (d, 2H), 7.22 (d, 2H), 5.12 (s, 2H), 4.26 (q, 2H), 4.01 (t, 2H), 2.72 (q, 2H), 1.81-1.70 (m, 2H), 1.40 (t, 3H), 1.31 (t, 3H), 0.93 (t, 3H).

HRMS Calcd for [C₁₉H₂₃ClN₄O₂+H]⁺: 375.159. Found: 375.158.

### Example 73

### 1-(4-chlorobenzyl)-8-ethyl-7-isopropyl-3-propyl-3,7-dihydro-1H-purine-2,6-dione

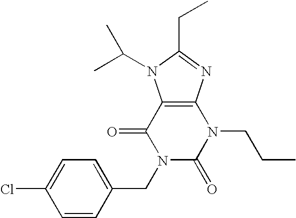

From 28 mg (0.106 mmol) 8-ethyl-7-isopropyl-3-propyl-3,7-dihydro-1H-purine-2,6-dione using potassium carbonate as a base, 23 mg (0.059 mmol, 56%) of the title compound was isolated.

¹H NMR (400 MHz, CDCl₃) δ 7.40 (d, 2H), 7.22 (d, 2H), 5.14 (s, 2H), 4.80-4.60 (br, 1H), 4.01 (t, 2H), 2.78 (q, 2H), 1.81-1.70 (m, 2H), 1.59 (d, 6H), 1.29 (t, 3H), 0.93 (t, 3H).

HRMS Calcd for [C₂₀H₂₅ClN₄O₂+H]⁺: 389.174. Found: 389.173.

The following compounds were synthesized in an analogous manner/method to example 10:

### Example 74

### 1-(4-Chlorobenzyl)-8-ethyl-7-(4-fluorophenyl)-3-propyl-3,7-dihydro-1H-purine-2,6-dione

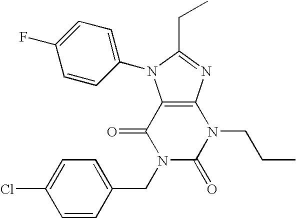

From 200 mg (0.5 mmol) 1-(4-chlorobenzyl)-8-ethyl-7-(4-fluorophenyl)-3,7-dihydro-1H-purine-2,6-dione using potassium carbonate as a base, 148 mg (0.335 mmol, 67%) of the title compound was isolated.

¹H NMR (400 MHz, CDCl₃) δ 7.36 (d, 2H), 7.33-7.26 (m, 2H), 7.22-7.17 (m, 4H), 5.05 (s, 2H), 4.12-4.04 (m, 2H), 3.59 (q, 2H), 1.87-1.75 (m, 2H), 1.21 (t, 3H), 0.98 (t, 3H).

HRMS Calcd for [C₂₃H₂₂ClFN₄O₂+H]⁺: 441.149. Found: 441.148.

### Example 75

### Synthesis of 8-methoxy-7-methyl-3-(3,3,3-trifluoropropyl)-3,7-dihydro-1H-purine-2,6-dione

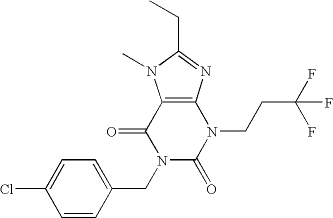

4-Chlorobenzyl isocyanate (274 μl, 1.88 mmol) was added to a solution of ethyl 2-ethyl-1-methyl-4-[(3,3,3-trifluoropropyl)amino]-1H-imidazole-5-carboxylate (250 mg, 0.85 mmol) in 1,2-dichloroethane (4 mL). The mixture was heated to 130° C. for 1 h using microwave heating. The solvent was evaporated. NaOMe (5.11 mL, 0.25 M in methanol) was added. The mixture was heated under reflux for 2 hours. The reaction was quenched with acetic acid (aq), DCM and water were added and the phases separated. The organic phase was dried by filtration through a phase separator. The product was purified by preparatory HPLC. 210 mg (0.51 mmol, 59%) of the title compound were isolated.

¹H NMR (400 MHz, CDCl₃) δ 7.38 (d, 2H), 7.23 (d, 2H), 5.10 (s, 2H), 4.33 (t, 2H), 3.87 (s, 3H), 2.71 (q, 2H), 2.66-2.53 (m, 2H), 1.31 (t, 3H).

HRMS Calcd for [C₁₈H₁₈ClF₃N₄O₂+H]⁺: 415.115. Found: 415.115.

The following compound was synthesized according to example 72:

### Example 76

### 8-Methoxy-7-methyl-3-(4,4,4-trifluorobutyl)-3,7-dihydro-1H-purine-2,6-dione

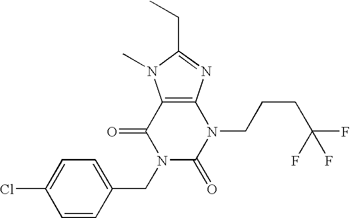

From 257 mg (0.84 mmol) ethyl 2-ethyl-1-methyl-4-[(4,4,4-trifluorobutyl)amino]-1H-imidazole-5-carboxylate, 176 mg (0.41 mmol, 49%) of the title compound were isolated.

¹H NMR (400 MHz, CDCl₃) δ 7.39 (d, 2H), 7.23 (d, 2H), 5.11 (s, 2H), 4.13 (t, 2H), 3.87 (s, 3H), 3.71 (q, 2H), 2.23-2.07 (m, 2H), 2.06-1.96 (m, 2H), 1.30 (t, 3H).

HRMS Calcd for [C₁₉H₂₀ClF₃N₄O₂+H]⁺: 429.130. Found: 429.130.

### Example 77

### Synthesis of 1-(4-chlorobenzyl)-3-ethyl-7-(4-fluorophenyl)-8-methoxy-3,7-dihydro-1H-purine-2,6-dione

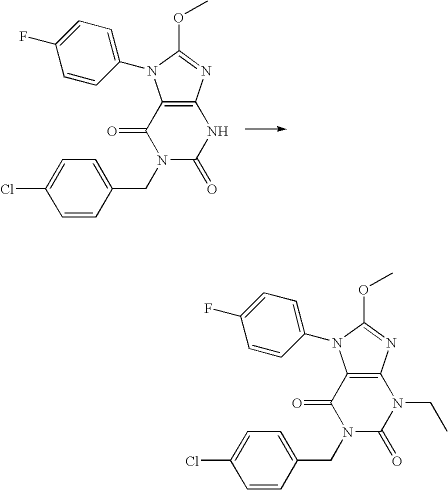

24 mg (60 μmol) of 1-(4-chlorobenzyl)-7-(4-fluorophenyl)-8-methoxy-3,7-dihydro-1H-purine-2,6-dione were dissolved in 1 mL DMF, then 39 mg (0.12 mmol) Cs₂CO₃ were added, followed by 14 mg (90 μmol) of iodoethane. The resulting mixture was stirred at room temperature for 30 min, then ethyl acetate was added and the organic layer was washed with water and brine. The organic layer was dried over Na₂SO₄ and evaporated. 25 mg (58 μmol, 97%) of the title compound was isolated.

¹H NMR (500 MHz, CDCl₃) δ 7.38 (d, 8.5 Hz, 2H), 7.32-7.36 (m, 2H), 7.20 (d, 8.5 Hz, 2H), 7.11-7.16 (m, 2H), 5.07 (s, 2H), 4.10-4.17 (m, 5H), 1.34 (t, 7.1 Hz, 3H).

HRMS Calcd for [C₂₁H₁₈ClFN₄O₃+H]⁺: 22222. Found: 22222.

### Example 78

### Synthesis of 1-(4-Chlorobenzyl)-8-methoxy-7-methyl-3-propyl-3,7-dihydro-1H-purine-2,6-dione

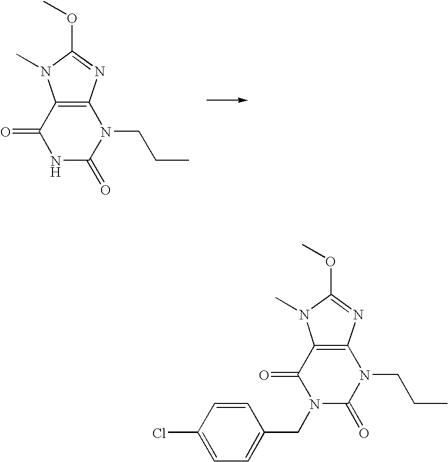

8-Methoxy-7-methyl-3-propyl-3,7-dihydro-1H-purine-2,6-dione (47 mg, 0.197 mmol) was dissolved in 2 mL DMF and cesium carbonate (257 mg, 0.789 mmol) and tetrabutylammonium iodide (2 mg, 0.006 mmol) was added. The reaction mixture was stirred for a few minutes at rt, then 4-chlorobenzylbromide (57 mg, 0.277 mmol) was added. The reaction mixture was stirred for 2 h at rt. EtOAc was added to the reaction mixture and washed twice with water. The organic layer was dried by filtration through a phase separator and evaporated. The crude was purified by preparative HPLC and 40 mg (0.11 mmol, 55%) of the title compound was isolated.

¹H-NMR (400 MHz, CDCl₃) δ 7.46-7.41 (m, 2H), 7.29-7.24 (m, 2H), 5.14 (s, 2H), 4.13 (s, 3H), 4.00 (t, 2H), 3.69 (s, 3H), 1.82-1.73 (m, 2H), 0.96 (t, 3H)

MS m/z 363.1229 (M+H)⁺

The following compounds were synthesized according to example 78:

### Example 79

### 1-(4-Fluorobenzyl)-8-methoxy-7-methyl-3-propyl-3,7-dihydro-1H-purine-2,6-dione

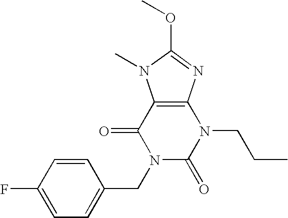

From 560 mg (2.35 mmol) 8-methoxy-7-methyl-3-propyl-3,7-dihydro-1H-purine-2,6-dione, 38 mg (0.108 mmol, 4.6%) of the title compound was isolated.

¹H-NMR (400 MHz, CDCl₃) δ 7.52-7.47 (m, 2H), 7.01-6.95 (m, 2H), 5.15 (s, 2H), 4.15 (s, 3H), 4.01 (t, 2H), 3.70 (s, 3H), 1.82-1.73 (m, 2H), 0.96 (t, 3H)

MS m/z 347.153 (M+H)⁺

### Example 80

### 1-(4-chlorobenzyl)-8-methoxy-7-methyl-3-(3,3,3-trifluoropropyl)-3,7-dihydro-1H-purine-2,6-dione

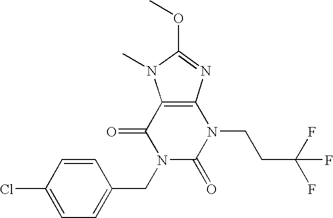

From 92 mg (0.315 mmol) 8-methoxy-7-methyl-3-(3,3,3-trifluoropropyl)-3,7-dihydro-1H-purine-2,6-dione using potassium carbonate as a base, 45 mg (0.108 mmol, 34%) of the title compound was isolated.

¹H NMR (400 MHz, CDCl₃) δ 7.37 (d, 2H), 7.22 (d, 2H), 5.08 (s, 2H), 4.27 (t, 2H), 4.09 (s, 3H), 3.65 (s, 3H), 2.64-2.50 (m, 2H).

HRMS Calcd for [C₁₇H₁₆ClF₃N₄O₃+H]⁺: 417.094. Found: 417.092.

### Example 81

### 1-(4-chlorobenzyl)-8-methoxy-7-methyl-3-(4,4,4-trifluorobutyl)-3,7-dihydro-1H-purine-2,6-dione

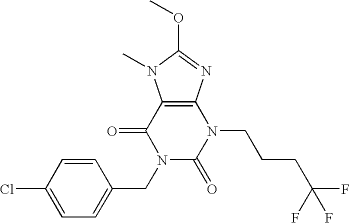

From 262 mg (0.856 mmol) 8-methoxy-7-methyl-3-(4,4,4-trifluorobutyl)-3,7-dihydro-1H-purine-2,6-dione using potassium carbonate as a base, 64 mg (0.149 mmol, 17%) of the title compound was isolated

¹H NMR (400 MHz, CDCl₃) δ 7.38 (d, 2H), 7.23 (d, 2H), 5.09 (s, 2H), 4.12-4.04 (m, 5H), 3.66 (s, 3H), 2.22-2.08 (m, 2H), 2.05-1.95 (m, 2H).

HRMS Calcd for [C₁₈H₁₈ClF₃N₄O₃+H]⁺: 431.110. Found: 431.111.

### Example 82

### Synthesis of 1-(4-chlorobenzyl)-8-(dimethylamino)-7-methyl-3-propyl-3,7-dihydro-1H-purine-2,6-dione

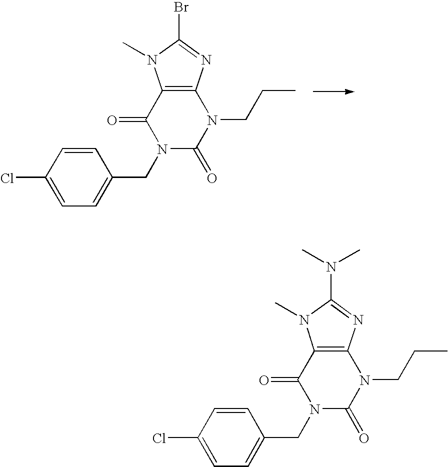

54 mg (0.131 mmol) 8-bromo-1-(4-chlorobenzyl)-7-methyl-3-propyl-3,7-dihydro-1H-purine-2,6-dione was suspended in 2 mL 2M dimethylamine in methanol (4 mmol) and was heated to 120° C. for 4 h in a closed vial using microwave heating. The solvents were evaporated and the residue purified by reversed phase HPLC. 33 mg (88 □mol, 67%) 1-(4-chlorobenzyl)-8-(dimethylamino)-7-methyl-3-propyl-3,7-dihydro-1H-purine-2,6-dione was isolated as a colorless oil.

¹H NMR (400 MHz, CDCl₃) δ 7.39 (d, 8.5 Hz, 2H), 7.23 (d, 8.5 Hz, 2H), 5.10 (s, 2H), 3.94-3.99 (m, 2H), 3.73 (s, 3H), 2.93 (s, 6H) 1.70-1.76 (m, 2H), 0.91 (t, 7.5 Hz, 3H).

HRMS Calcd for [C₁₈H₂₂ClN₅O₂+H]⁺: 376.1540. Found: 376.1535.

### Example 83

### Synthesis of 8-azetidin-1-yl-1-(4-chlorobenzyl)-7-methyl-3-propyl-3,7-dihydro-1H-purine-2,6-dione

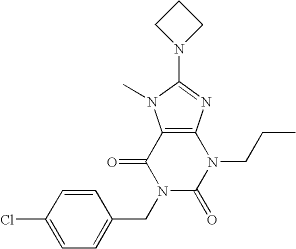

41 mg (0.1 mmol) 8-bromo-1-(4-chlorobenzyl)-7-methyl-3-propyl-3,7-dihydro-1H-purine-2,6-dione, 34.3 mg (0.6 mmol) azetidine and 78 mg (0.6 mmol) DIPEA were mixed in 1 mL ethanol and the resulting mixture was heated to 120° C. for 1 h in a sealed vial using microwave heating. Then dichloromethane was added to the reaction mixture and was washed with sat. NaHCO₃. The organic layer was dried by filtration through a phase separator. The solvents were evaporated and the residue purified by reversed phase HPLC. 14 mg (0.037 mmol, 37%) of the title compound was isolated.

¹H-NMR (600 MHz, (CH₃)₂SO*, (CD₃)₂SO)) δ 7.31-7.34 (m, 2H), 7.23-7.26 (m, 2H), 4.96 (s, 2H), 4.16 (t, 7.6 Hz, 4H), 3.82-3.86 (m, 2H), 3.58 (s, 3H), 2.29-2.35 (m, 2H) 1.58-1.66 (m, 2H), 0.81 (t, 7.5 Hz, 3H).

HRMS Calcd for [C₁₉H₂₂ClN₅O₂+H]⁺: 388.1540. Found: 388.1540.

The following compounds were synthesized according to example 83:

### Example 84

### 1-(4-chlorobenzyl)-8-(4-methoxypiperidin-1-yl)-7-methyl-3-propyl-3,7-dihydro-1H-purine-2,6-dione

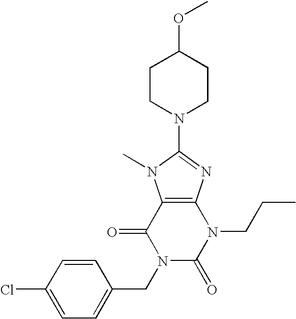

From 41 mg (0.1 mmol) 8-bromo-1-(4-chlorobenzyl)-7-methyl-3-propyl-3,7-dihydro-1H-purine-2,6-dione, 23 mg (0.052 mmol, 52%) of the title compound was isolated.

¹H-NMR (600 MHz, (CH₃)₂SO*, (CD₃)₂SO)) δ 7.31-7.34 (m, 2H), 7.24-7.27 (m, 2H), 4.98 (s, 2H), 3.84-3.88 (m, 2H), 3.62 (s, 3H), 3.34-3.42 (m, 3H), 3.24 (s, 3H), 3.00-3.05 (m, 2H), 1.89-1.94 (m, 2H), 1.61-1.65 (m, 2H), 1.52-1.58 (m, 2H), 0.81 (t, 7.5 Hz, 3H).

HRMS Calcd for [C₂₂H₂₈ClN₅O₃+H]⁺: 446.1959. Found: 446.1968.

### Example 85

### 1-(4-chlorobenzyl)-7-methyl-8-piperidin-1-yl-3-propyl-3,7-dihydro-1H-purine-2,6-dione

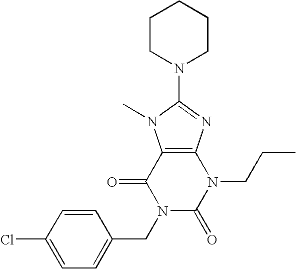

From 41 mg (0.1 mmol) 8-bromo-1-(4-chlorobenzyl)-7-methyl-3-propyl-3,7-dihydro-1H-purine-2,6-dione, 21 mg (0.051 mmol, 51%) of the title compound was isolated.

¹H-NMR (600 MHz, (CH₃)₂SO*, (CD₃)₂SO)) δ 7.31-7.34 (m, 2H), 7.24-7.27 (m, 2H), 4.98 (s, 2H), 3.84-3.88 (m, 2H), 3.61 (s, 3H), 3.16-3.18 (m, 4H), 1.52-1.67 (m, 8H), 0.81 (t, 7.5 Hz, 3H).

HRMS Calcd for [C₂₁H₂₆ClN₅O₂+H]⁺: 416.1853. Found: 416.1858.

### Example 86

### 1-(4-chlorobenzyl)-7-methyl-3-propyl-8-pyrrolidin-1-yl-3,7-dihydro-1H-purine-2,6-dione

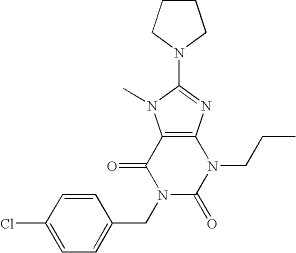

From 41 mg (0.1 mmol) 8-bromo-1-(4-chlorobenzyl)-7-methyl-3-propyl-3,7-dihydro-1H-purine-2,6-dione, 20.6 mg (0.051 mmol, 51%) of the title compound was isolated.

¹H-NMR (600 MHz, (CH₃)₂SO*, (CD₃)₂SO)) δ 7.31-7.34 (m, 2H), 7.23-7.26 (m, 2H), 4.97 (s, 2H), 3.83-3.86 (m, 2H), 3.76 (s, 3H), 3.53-3.56 (m, 4H), 1.84-1.88 (m, 4H), 1.59-1.66 (m, 2H), 0.81 (t, 7.5 Hz, 3H).

HRMS Calcd for [C₂₀H₂₄ClN₅O₂+H]⁺: 402.1697. Found: 402.1687.

### Example 87

### 1-(4-chlorobenzyl)-7-methyl-8-(4-methylpiperazin-1-yl)-3-propyl-3,7-dihydro-1H-purine-2,6-dione

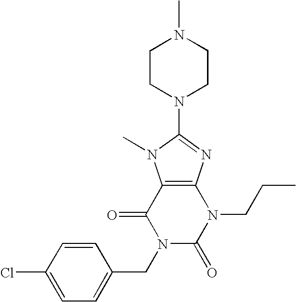

From 41 mg (0.1 mmol) 8-bromo-1-(4-chlorobenzyl)-7-methyl-3-propyl-3,7-dihydro-1H-purine-2,6-dione, 21 mg (0.049 mmol, 49%) of the title compound was isolated.

¹H-NMR (600 MHz, (CH₃)₂SO*, (CD₃)₂SO)) δ 7.31-7.33 (m, 2H), 7.24-7.26 (m, 2H), 4.98 (s, 2H), 3.85-3.88 (m, 2H), 3.63 (s, 3H), 3.20-3.22 (m, 4H), 2.42-2.44 (m, 4H), 2.20 (s, 3H), 1.61-1.65 (m, 2H), 0.81 (t, 7.5 Hz, 3H).

HRMS Calcd for [C₂₁H₂₇ClN₆O₂+H]⁺: 431.1962. Found: 431.1954.

### Example 88

### 1-(4-chlorobenzyl)-7-methyl-3-propyl-8-thiomorpholin-4-yl-3,7-dihydro-1H-purine-2,6-dione

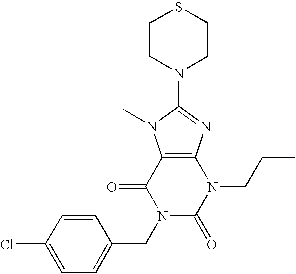

From 41 mg (0.1 mmol) 8-bromo-1-(4-chlorobenzyl)-7-methyl-3-propyl-3,7-dihydro-1H-purine-2,6-dione, 16 mg (0.038 mmol, 38%) of the title compound was isolated.

¹H-NMR (600 MHz, (CH₃)₂SO*, (CD₃)₂SO)) δ 7.31-7.34 (m, 2H), 7.24-7.27 (m, 2H), 4.98 (s, 2H), 3.85-3.88 (m, 2H), 3.62 (s, 3H), 3.45-3.47 (m, 4H), 2.71-2.74 (m, 4H), 1.61-1.66 (m, 2H), 0.81 (t, 7.5 Hz, 3H).

HRMS Calcd for [C₂₀H₂₄ClN₅O₂S+H]⁺: 434.1417. Found: 434.1400.

### Example 89

### 1-(4-chlorobenzyl)-8-(diethylamino)-7-methyl-3-propyl-3,7-dihydro-1H-purine-2,6-dione

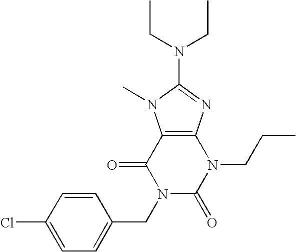

From 41 mg (0.1 mmol) 8-bromo-1-(4-chlorobenzyl)-7-methyl-3-propyl-3,7-dihydro-1H-purine-2,6-dione, 1.7 mg (0.004 mmol, 4%) of the title compound was isolated.

HRMS Calcd for [C₂₀H₂₆ClN₅O₂+H]⁺: 404.1853. Found: 404.1861.

### Example 90

### 1-(4-chlorobenzyl)-8-[(3R)-3-(dimethylamino)pyrrolidin-1-yl]-7-methyl-3-propyl-3,7-dihydro-1H-purine-2,6-dione

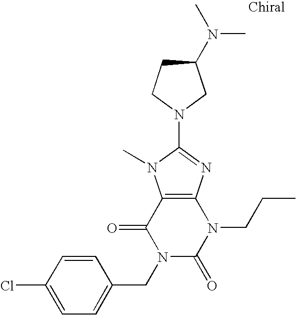

From 41 mg (0.1 mmol) 8-bromo-1-(4-chlorobenzyl)-7-methyl-3-propyl-3,7-dihydro-1H-purine-2,6-dione, 20 mg (0.044 mmol, 44%) of the title compound was isolated.

¹H-NMR (600 MHz, (CH₃)₂SO*, (CD₃)₂SO)) δ 7.31-7.35 (m, 2H), 7.23-7.26 (m, 2H), 4.97 (s, 2H), 3.83-3.86 (m, 2H), 3.76 (s, 3H), 3.70-3.74 (m, 1H), 3.62-3.65 (m, 1H), 3.56-3.60 (m, 1H), 2.16 (s, 6H), 1.60-1.66 (m, 2H), 0.81 (t, 7.5 Hz, 3H).

HRMS Calcd for [C₂₂H₂₉ClN₆O₂+H]⁺: 445.2119. Found: 445.2108.

### Example 91

### 1-(4-chlorobenzyl)-8-[(2-methoxyethyl)(methyl)amino]-7-methyl-3-propyl-3,7-dihydro-1H-purine-2,6-dione

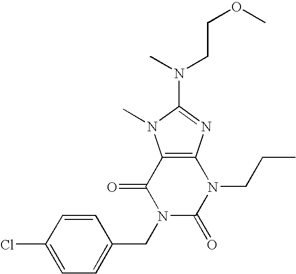

From 41 mg (0.1 mmol) 8-bromo-1-(4-chlorobenzyl)-7-methyl-3-propyl-3,7-dihydro-1H-purine-2,6-dione, 18 mg (0.042 mmol, 42%) of the title compound was isolated.

¹H-NMR (600 MHz, (CH₃)₂SO*, (CD₃)₂SO)) δ 7.31-7.34 (m, 2H), 7.24-7.27 (m, 2H), 4.98 (s, 2H), 3.84-3.87 (m, 2H), 3.68 (s, 3H), 3.51-3.54 (m, 2H), 3.42-3.45 (m, 2H), 3.22 (s, 3H), 2.98 (s, 3H), 1.60-1.65 (m, 2H), 0.81 (t, 7.5 Hz, 3H).

HRMS Calcd for [C₂₀H₂₆ClN₅O₃+H]⁺: 420.1802. Found: 420.1774.

### Example 92

### 1-(4-chlorobenzyl)-7-methyl-8-morpholin-4-yl-3-propyl-3,7-dihydro-1H-purine-2,6-dione

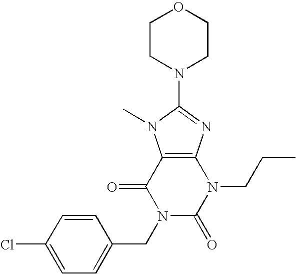

From 41 mg (0.1 mmol) 8-bromo-1-(4-chlorobenzyl)-7-methyl-3-propyl-3,7-dihydro-1H-purine-2,6-dione, 20.2 mg (0.048 mmol, 48%) of the title compound was isolated.

¹H-NMR (600 MHz, (CH₃)₂SO*, (CD₃)₂SO)) δ 7.31-7.34 (m, 2H), 7.24-7.27 (m, 2H), 4.99 (s, 2H), 3.85-3.88 (m, 2H), 3.69-3.71 (m, 4H), 3.66 (s, 3H), 3.19-3.21 (m, 4H), 1.61-1.66 (m, 2H), 0.81 (t, 7.5 Hz, 3H).

HRMS Calcd for [C₂₀H₂₄ClN₅O₃+H]⁺: 418.1646. Found: 418.1651.

### Example 93

### 1-(4-chlorobenzyl)-8-[(2S)-2-(methoxymethyl)pyrrolidin-1-yl]-7-methyl-3-propyl-3,7-dihydro-1H-purine-2,6-dione

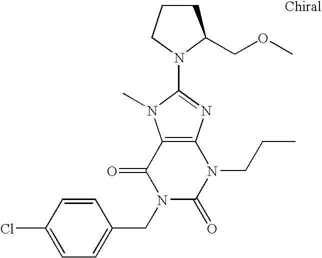

From 41 mg (0.1 mmol) 8-bromo-1-(4-chlorobenzyl)-7-methyl-3-propyl-3,7-dihydro-1H-purine-2,6-dione, 23 mg (0.051 mmol, 51%) of the title compound was isolated.

¹H-NMR (600 MHz, (CH₃)₂SO*, (CD₃)₂SO)) δ 7.31-7.34 (m, 2H), 7.24-7.27 (m, 2H), 4.97 (s, 2H), 4.15-4.30 (m, 1H), 3.81-3.90 (m, 2H), 3.72 (s, 3H), 3.66-3.71 (m, 1H), 3.42-3.50 (m, 2H), 3.22 (s, 2H), 1.91-2.02 (m, 2H), 1.71-1.91 (m, 2H), 1.63-1.67 (m, 2H), 0.81 (t, 7.5 Hz, 3H).

HRMS Calcd for [C₂₂H₂₈ClN₅O₃+H]⁺: 446.1959. Found: 446.1964.

### Example 94

### 1-(4-chlorobenzyl)-7-methyl-3-propyl-8-[(2S)-2-(pyrrolidin-1-ylmethyl)pyrrolidin-1-yl]-3,7-dihydro-1H-purine-2,6-dione

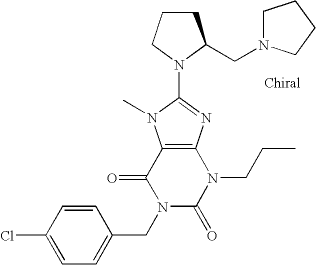

From 41 mg (0.1 mmol) 8-bromo-1-(4-chlorobenzyl)-7-methyl-3-propyl-3,7-dihydro-1H-purine-2,6-dione, 24 mg (0.049 mmol, 49%) of the title compound was isolated.

¹H-NMR (600 MHz, (CH₃)₂SO*, (CD₃)₂SO)) δ 7.31-7.33 (m, 2H), 7.24-7.27 (m, 2H), 4.97 (s, 2H), 4.21-4.25 (m, 1H), 3.79-3.87 (m, 1H), 3.68-3.73 (m, 4H), 3.43-3.48 (m, 1H), 2.00-2.04 (m, 1H), 1.88-2.00 (m, 1H), 1.70-1.82 (m, 2H), 1.59-1.67 (m, 7H), 0.82 (t, 7.5 Hz, 3H).

HRMS Calcd for [C₂₅H₃₃ClN₆O₂+H]⁺: 485.2432. Found: 485.2419.

### Example 95

### Synthesis of 8-bromo-1-(4-chlorobenzyl)-7-methyl-3-propyl-3,7-dihydro-1H-purine-2,6-dione

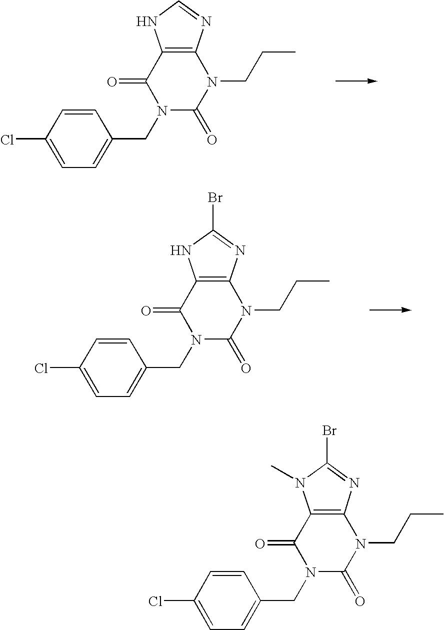

810 mg (2.41 mmol) 1-(4-chlorobenzyl)-3-propyl-3,7-dihydro-1H-purine-2,6-dione was suspended in 30 mL acetic acid, then 355 mg (3.62 mmol) potassium acetate was added. The resulting mixture was warmed to 45° C., then 451 mg (2.82 mmol) of bromine was added dropwise. The resulting mixture was stirred at 45° C. overnight. The reaction mixture was cooled to room temperature, then the solvents evaporated. The residue was partitioned between ethyl acetate and water. After phase separation the organic layer was dried over MgSO₄ and evaporated. The residue was dissolved in 15 mL DMF, then 730 mg (5.28 mmol) of K₂CO₃ and 546 mg (3.84 mmol) of iodomethane was added. The reaction mixture was stirred at room temperature overnight, then diluted with ethylacetate (ca. 150 mL) and washed with water, sat. NaHCO₃ and brine. The organic layer was dried and evaporated. 990 mg (2.16 mmol, ca. 90% pure, 94%) 8-bromo-1-(4-chlorobenzyl)-7-methyl-3-propyl-3,7-dihydro-1H-purine-2,6-dione was isolated as off-white solid that was used without further purification. A small amount was purified by reversed phase HPLC.

¹H NMR (400 MHz, CDCl₃) δ 7.39 (d, 8.5 Hz, 2H), 7.23 (d, 8.5 Hz, 2H), 5.11 (s, 2H), 3.97-4.02 (m, 2H), 3.92 (s, 3H), 1.71-1.77 (m, 2H), 0.93 (t, 7.5 Hz, 3H).

HRMS Calcd for [C₁₆H₁₆BrClN₄O₂+H]⁺: 411.0223. Found: 411.0251.

### Example 96

### Synthesis of 1-(4-chlorobenzyl)-8-(1-hydroxyethyl)-7-methyl-3-propyl-3,7-dihydro-1H-purine-2,6-dione

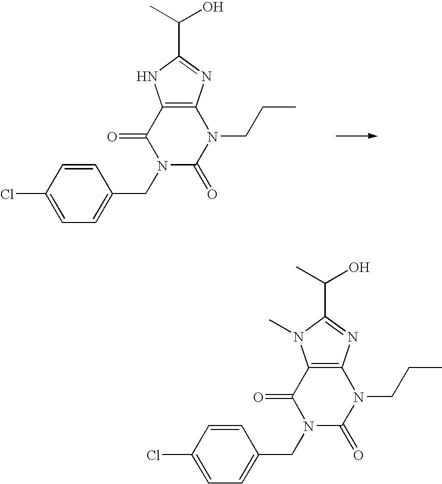

60 mg (0.165 mmol) 1-(4-chlorobenzyl)-8-(1-hydroxyethyl)-3-propyl-3,7-dihydro-1H-purine-2,6-dione was dissolved in DMF, then 54 mg (0.39 mmol) K₂CO₃ and 28 mg (0.198 mmol) iodomethane were added. The resulting mixture was stirred at room temperature for 2 h. The solids were filtered off and the filtrate purified by reversed phase HPLC. 56 mg (0.149 mmol, 90%) 1-(4-chlorobenzyl)-8-(1-hydroxyethyl)-7-methyl-3-propyl-3,7-dihydro-1H-purine-2,6-dione was isolated as a colorless solid.

¹H NMR (500 MHz, CDCl₃) δ 7.37 (d, 8.5 Hz, 2H), 7.21 (d, 8.5 Hz, 2H), 5.09 (s, 2H), 4.94 (q, 6.6 Hz, 1H), 3.94-3.99 (m, 5H), 2.04 (s, 1H), 1.65-1.74 (m, 2H), 1.57 (d, 6.6 Hz, 3H), 0.90 (t, 7.5 Hz, 3H).

HRMS Calcd for [C₁₈H₂₁ClN₄O₃+H]⁺: 377.1380. Found: 377.1387.

These compounds were not screened in the PAM screen:

### Example 97

### Synthesis of 1-(4-chlorobenzyl)-7,8-dimethyl-3-propyl-3,7-dihydro-1H-purine-2,6-dione

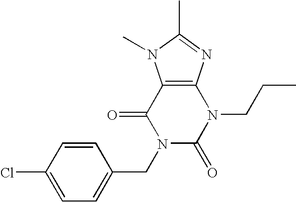

92 mg (0.3 mmol) 5,6-diamino-3-(4-chlorobenzyl)-1-propylpyrimidine-2,4(1H,3H)-dione was dissolved in 1.5 mL DMF, then acetic acid (36 mg, 0.6 mmol) was added, followed by EDC HCl (115 mg, 0.6 mmol), DMAP (12 mg, 0.1 mmol) and DIPEA (65 mg, 0.5 mmol). The resulting mixture was stirred at room temperature overnight, then NaOH (120 mg, 3 mmol) in 1 mL water/EtOH 1:1 was added. The resulting mixture was heated to 100° C. for 8 h. The reaction mixture was partitioned between DCM and 1N HCl and then filtered through a phase separator. The aqueous layer was extracted with DCM and then the organic layer was filtered through a phase separator. The combined organic layers were evaporated.

The residue was dissolved in 3 mL DMSO/DMF 1:2 and K₂CO₃ (415 mg, 3 mmol) was added. Then iodomethane (227 mg, 1.6 mmol) was added and the resulting mixture was stirred at room temperature overnight. Water and dichloromethane were added to the reaction mixture. The organic layer was filtered through a phase separator and the aqueous layer extracted again with dichloromethane. The organic layer was filtered through a phase separator. The combined organic layers were evaporated and the residue purified by reversed phase HPLC. 56 mg (0.162 mmol, 54%) of the title compound were isolated.

¹H-NMR (600 MHz, (CH₃)₂SO*, (CD₃)₂SO)) δ 7.32-7.34 (m, 2H), 7.25-7.28 (m, 2H), 5.00 (s, 2H), 3.88-3.92 (m, 2H), 3.78 (s, 3H), 2.38 (s, 3H), 1.61-1.65 (m, 2H), 0.83 (t, 7.3 Hz, 3H)

HRMS Calcd for [C₁₇H₁₉ClN₄O₂+H]⁺: 347.1275. Found: 347.1293.

The following compounds were synthesized according to example 97:

### Example 98

### 1-(4-chlorobenzyl)-7-methyl-3-propyl-8-(tetrahydrofuran-3-yl)-3,7-dihydro-1H-purine-2,6-dione

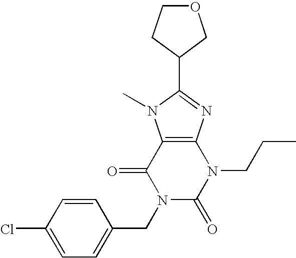

From 92 mg (0.3 mmol) 5,6-diamino-3-(4-chlorobenzyl)-1-propylpyrimidine-2,4(1H,3H)-dione, 79 mg (0.198 mmol, 66%) of the title compound were isolated.

¹H-NMR (600 MHz, (CH₃)₂SO*, (CD₃)₂SO)) δ 7.32-7.34 (m, 2H), 7.25-7.28 (m, 2H), 5.00 (s, 2H), 4.02-4.06 (m, 1H), 3.85-3.92 (m, 3H), 3.84 (s, 3H), 3.74-3.82 (m, 2H), 3.65-3.70 (m, 1H), 2.23-2.30 (m, 1H), 2.10-2.16 (m, 1H), 1.61-1.68 (m, 2H), 0.83 (t, 7.3 Hz, 3H)

HRMS Calcd for [C₂₀H₂₃ClN₄O₃+H]⁺: 403.1537. Found: 403.1545.

### Example 99

### 1-(4-chlorobenzyl)-8-cyclohexyl-7-methyl-3-propyl-3,7-dihydro-1H-purine-2,6-dione

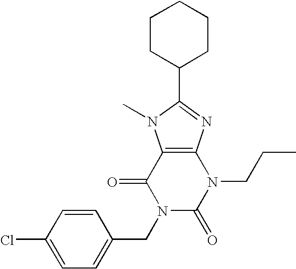

From 92 mg (0.3 mmol) 5,6-diamino-3-(4-chlorobenzyl)-1-propylpyrimidine-2,4(1H,3H)-dione, 37 mg (0.089 mmol, 30%) of the title compound were isolated.

¹H-NMR (600 MHz, (CH₃)₂SO*, (CD₃)₂SO)) δ 7.32-7.34 (m, 2H), 7.25-7.28 (m, 2H), 5.00 (s, 2H), 3.88-3.92 (m, 2H), 3.83 (s, 3H), 2.82-2.88 (m, 1H), 1.72-1.84 (m, 4H), 1.61-1.68 (m, 3H), 1.46-1.54 (m, 2H), 1.31-1.40 (m, 2H), 1.20-1.28 (m, 1H), 0.83 (t, 7.3 Hz, 3H)

HRMS Calcd for [C₂₂H₂₇ClN₄O₂+H]⁺: 415.1901. Found: 415.1913.

### Example 100

### 1-(4-chlorobenzyl)-8-(methoxymethyl)-7-methyl-3-propyl-3,7-dihydro-1H-purine-2,6-dione

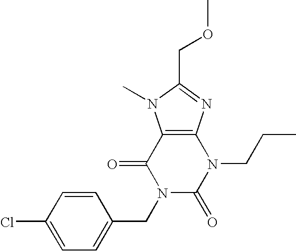

From 92 mg (0.3 mmol) 5,6-diamino-3-(4-chlorobenzyl)-1-propylpyrimidine-2,4(1H,3H)-dione, 40 mg (0.107 mmol, 36%) of the title compound were isolated.

¹H-NMR (600 MHz, (CH₃)₂SO*, (CD₃)₂SO)) δ 7.32-7.35 (m, 2H), 7.27-7.30 (m, 2H), 5.02 (s, 2H), 4.55 (s, 2H), 3.89-3.93 (m, 2H), 3.86 (s, 3H), 1.60-1.68 (m, 2H), 0.83 (t, 7.3 Hz, 3H)

HRMS Calcd for [C₁₈H₂₁ClN₄O₃+H]⁺: 377.1380. Found: 377.1400.

### Example 101

### 1-(4-chlorobenzyl)-8-cyclopentyl-7-methyl-3-propyl-3,7-dihydro-1H-purine-2,6-dione

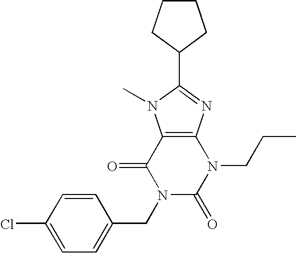

From 92 mg (0.3 mmol) 5,6-diamino-3-(4-chlorobenzyl)-1-propylpyrimidine-2,4(1H,3H)-dione, 45 mg (0.111 mmol, 37%) of the title compound were isolated.

¹H-NMR (600 MHz, (CH₃)₂SO*, (CD₃)₂SO)) δ 7.32-7.34 (m, 2H), 7.25-7.28 (m, 2H), 5.00 (s, 2H), 3.88-3.94 (m, 2H), 3.85 (s, 3H), 1.94-2.02 (m, 2H), 1.70-1.80 (m, 4H), 1.58-1.68 (m, 4H), 0.83 (t, 7.3 Hz, 3H)

HRMS Calcd for [C₂₁H₂₅ClN₄O₂+H]⁺: 401.1744. Found: 401.1766.

### Example 102

### 1-(4-chlorobenzyl)-7-methyl-8-(5-oxopyrrolidin-2-yl)-3-propyl-3,7-dihydro-1H-purine-2,6-dione

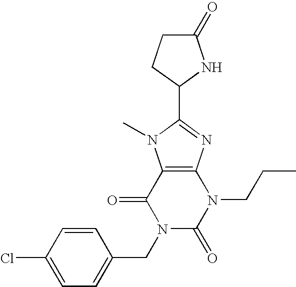

From 92 mg (0.3 mmol) 5,6-diamino-3-(4-chlorobenzyl)-1-propylpyrimidine-2,4(1H,3H)-dione, 45 mg (0.107 mmol, 36%) of the title compound were isolated.

¹H-NMR (600 MHz, (CH₃)₂SO*, (CD₃)₂SO)) δ 8.08 (s, 1H), 7.32-7.35 (m, 2H), 7.25-7.28 (m, 2H), 5.01 (s, 2H), 4.94-4.97 (m, 1H), 3.89-3.94 (m, 2H), 3.88 (s, 3H), 2.16-2.44 (m, 4H), 1.60-1.68 (m, 2H), 0.83 (t, 7.3 Hz, 3H)

HRMS Calcd for [C₂₀H₂₂ClN₅O₃+H]⁺: 416.1489. Found: 416.1495.

### Example 103

### 1-(4-chlorobenzyl)-8-cyclobutyl-7-methyl-3-propyl-3,7-dihydro-1H-purine-2,6-dione

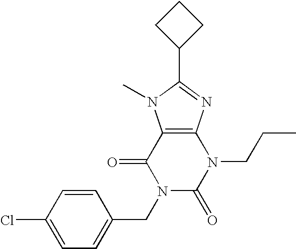

From 92 mg (0.3 mmol) 5,6-diamino-3-(4-chlorobenzyl)-1-propylpyrimidine-2,4(1H,3H)-dione, 52 mg (0.136 mmol, 45%) of the title compound were isolated.

¹H-NMR (600 MHz, (CH₃)₂SO*, (CD₃)₂SO)) δ 7.31-7.34 (m, 2H), 7.25-7.28 (m, 2H), 5.00 (s, 2H), 3.91-3.95 (m, 2H), 3.68-3.76 (m, 4H), 2.28-2.35, (m, 4H), 1.98-2.06 (m, 1H), 1.82-1.90 (m, 1H), 1.63-1.70 (m, 2H), 0.83 (t, 7.3 Hz, 3H)

HRMS Calcd for [C₂₀H₂₃ClN₄O₂+H]⁺: 387.1588. Found: 387.1592.

### Analysis

LC-MS analysis was performed using a Micromass 8 probe MUX-LTC ESP+ system, purity being determined by single wavelength (254 nm) UV detection.

Chromatography was performed over an Xterra™ MS C8 3.5 um, 4.6×30 mm column, 8 in parallel. The flow of 15 ml/min was split over the 8 columns to give a flow rate of 1.9 ml/min. The 10-minute chromatography gradient was as follows:

### Mobile Phase A: 95% ACN+5% 0.010 M NH₄OAc

### Mobile Phase B: 5% ACN+95% 0.010 M NH₄OAc

| | | |
|---|---|---|
| 10 min | 0.0 min | 0% A |
| | 8.0 min | 100% A |
| | 9.0 min | 100% A |
| | 9.1 min | 0% A |

NMR analysis was performed at 400 MHz.

### Biological Evaluation

### Effects of the Positive Allosteric GABA_{B} Receptor Modulator in a Functional In Vitro Assay.

The effect of GABA and baclofen on intracellular calcium release in CHO cells expressing the GABA_{B(1A,2)} receptor heterodimer was studied in the presence or absence of the positive allosteric modulator. The positive allosteric modulator according to the invention increased both the potency and the efficacy of GABA.

The potency of the compounds i.e. the ability of the compounds to reduce the EC₅₀ of GABA was revealed by the concentration required to reduce GABA's EC₅₀ by 50%. These potencies were similar to the potency reported for CGP7930 (can be purchased from Tocris, Northpoint, Fourth Way, Avonmouth, Bristol, BS11 8TA, UK) by Urwyler et al. CGP7930 increases the potency of GABA from EC₅₀ of about 170-180 nM to EC₅₀ of about 35-50 nM.

### Experimental Procedures

### Materials

Nut mix F-12 (Ham) cell culture media, OPTI-MEM I reduced serum medium, Fetal bovine serum (FBS), penicillin/streptomycin solution (PEST), geneticin, HEPES (4-(2-hydroxyethyl)-1-piperazineethanesulfonic acid (buffer), 1 M solution), Hank's Balanced Salt Solution (HBSS) and zeocin were from Life technologies (Paisley, Scotland); Polyethyleneimine, probenicid, baclofen and γ-aminobutyric acid (GABA) were from Sigma (St Louis, USA); Fluo-3 AM was from Molecular Probes (Oregon, USA). 4-Amino-n-[2,3-³H]butyric acid ([³H]GABA) was from Amersham Pharmacia Biotech (Uppsala, Sweden).

### Generation of Cell Lines Expressing the GABA_{B} Receptor

GABA_{B}R1a and GABA_{B}R2 were cloned from human brain cDNA and subcloned into pCI-Neo (Promega) and pALTER-1 (Promega), respectively. A GABA_{B}R1a-G_{αqi5} fusion protein expression vector was constructed using the pCI-Neo-GABA_{B}R1a cDNA plasmid and pLEC1-G_{αqi5} (Molecular Devices, CA). In order to make the G_{αqi5} pertussis toxin insensitive, Cys356 was mutated to Gly using standard PCR methodology with the primers 5′-GGATCCATGGCATGCTGCCTGAGCGA-3′ (forward) and 5′-GCGGCCG CTCAGAAGAGGCCGCCGTCCTT-3′ (reverse). The G_{αqi5mut} cDNA was ligated into the BamHI and NotI sites of pcDNA3.0 (Invitrogen). The GABA_{B} R1a coding sequence was amplified by PCR from pCI-Neo-GABA_{B}R1a using the primers, 5′-GGATCCCCGGGGAGCCGGGCCC-3′ (forward) and 5′-GGATCCCTTATAAAGCAAATGCACTCGA-3′ (reverse) and subcloned into the BamHI site of pcDNA3.0-G_{αqi5mut}.

In order to optimise the Kozak consensus sequence of GABA_{B}R2, in situ mutagenesis was performed using the Altered Sites Mutagenesis kit according to manufacturer's instruction (Promega) with the following primer, 5′-GAATTCGCACCATGGCTTCCC-3′. The optimised GABA_{B}R2 was then restricted from pALTER-1 with Xho I+Kpn I and subcloned into the mammalian expression vector pcDNA3.1(−)/Zeo (Invitrogen) to produce the final construct, pcDNA3.1(−)/Zeo-GABA_{B}R2.

For generation of stable cell lines, CHO-K1 cells were grown in Nut mix F-12 (Ham) media supplemented with 10% FBS, 100 U/ml Penicillin and 100 μg/ml Streptomycin at 37° C. in a humidified CO₂-incubator. The cells were detached with 1 mM EDTA in PBS and 1 million cells were seeded in 100 mm petri dishes. After 24 hours the culture media was replaced with OptiMEM and incubated for 1 hour in a CO₂-incubator.

For generation of a cell line expressing the GABA_{B}R1a/GABA_{B}R2 heterodimer, GABA_{B}R1a plasmid DNA (4 μg) GABA_{B}R2 plasmid DNA (4 μg) and lipofectamine (24 μl) were mixed in 5 ml OptiMEM and incubated for 45 minutes at room temperature. The cells were exposed to the transfection medium for 5 hours, which then was replaced with culture medium. The cells were cultured for an additional 10 days before selection agents (300 μg/ml hygromycin and 400 μg/ml geneticin) were added. Twenty-four days after transfection, single cell sorting into 96-well plates by flow cytometry was performed using a FACS Vantage SE (Becton Dickinson, Palo Alto, Calif.). After expansion, the GABA_{B} receptor functional response was tested using the FLIPR assay described below. The clone with the highest functional response was collected, expanded and then subcloned by single cell sorting. The clonal cell line with the highest peak response in the FLIPR was used in the present study.

For generation of a stable cell line expressing GABA_{B}R1a-G_{αqi5} fusion protein and GABA_{B}R2, GABA_{B}R1a-G_{αqi5mut} plasmid DNA (8 μg) GABA_{B}R2 plasmid DNA (8 μg) and lipofectamine (24 μl) were mixed in 5 ml OptiMEM and incubated for 45 minutes at room temperature. The cells were exposed to the transfection medium for 5 hours, which then was replaced with culture medium. After forty-eight hours, the cells were detached and seeded in 6 well plates (2000 cells/well) and grown in culture medium supplemented with geneticin (400 μg/ml) and zeocin (250 μg/ml). After 4 days, cells from single colonies were collected and transferred to a 24-well plate. After 10 days, the cell clones were seeded in T-25 flasks and grown for another 16 days before they were tested for GABA_{B} receptor mediated functional response. The clones that showed the highest peak response were collected and subcloned by seeding the cells in 6-well plates (1000 cells/well) and repeating the steps described above. The clonal cell line that gave the highest peak response in the FLIPR was used in the present study.

### Measurement of GABA_{B} Receptor Dependent Release of Intracellular Calcium in the FLIPR

Measurement of GABA_{B} receptor dependent release of intracellular calcium in the fluorescence imaging plate reader (FLIPR) was performed as described by Coward et al. Anal. Biochem. (1999) 270, 242-248, with some modifications. Transfected CHO cells were cultivated in Nut Mix F-12 (HAM) with Glutamax-I and supplemented with 10%, 100 U/ml penicillin and 100 μg/ml streptomycin, 250 μg/ml zeocin and 400 μg/ml geneticin. Twenty-four hours prior to the experiment the cells (35,000 cells/well) were seeded in black-walled 96-well poly-D-lysine coated plates (Becton Dickinson, Bedford, UK) in culture medium without selection agents. The cell culture medium was aspirated and 100 μl of Fluo-3 loading solution (4 μM Fluo-3, 2.5 mM probenecid and 20 mM Hepes in Nut Mix F-12 (Ham)) was added. After incubation for 1 hour at 37° C. in a 5% CO₂ incubator, the dye-solution was aspirated and the cells were washed 2 times with 150 μl of wash solution (2.5 mM probenecid and 20 mM Hepes in HBSS) followed by addition of 150 μl of wash solution. The cells were then assayed in a fluorescence imaging plate reader (Molecular Devices Corp., CA, USA). Test compounds were diluted to 50 μM concentrations in HBSS containing 20 mM Hepes and 5% DMSO and added in a volume of 50 μl. The fluorescence was sampled every second for 60 s (10 s before and 50 s after the addition of test compound) before GABA (50 μl 7.6 nM-150 M) was added and sampling continued every sixth second for additional 120 seconds.

### GTPgS

[³⁵S]-GTPγS binding assays were performed at 30° C. for 45 min in membrane buffer (100 mM NaCl, 5 mM MgCl₂, 1 mM EDTA, 50 mM HEPES, pH 7.4) containing 0.025 μg/μl of membrane protein (prepared from the cell lines described above) with 0.01% bovine serum albumin (fatty acid free), 10 μM GDP, 10 μM DTT and 0.53 nM [³⁵S]-GTPγS (Amersham-Pharmacia Biotech) in a final volume of 200 μl. Non-specific binding was determined in the presence of 20 μM GTPγS. The reaction was started by the addition of GABA at concentration between 1 mM and 0.1 nM in the presence or absence of the required concentration of PAM. The reaction was terminated by addition of ice-cold wash buffer (50 mM Tris-HCl, 5 mM MgCl₂, 50 mM NaCl, pH 7.4) followed by rapid filtration under vacuum through Printed Filtermat A glass fiber filters (Wallac) (0.05% PEI treated) using a Micro 96 Harvester (Skatron Instruments). The filters were dried for 30 min at 50° C., then a paraffin scintillant pad was melted onto the filters and the bound radioactivity was determined using a 1450 Microbeta Trilux (Wallac) scintillation counter.

### Calculations

GABA dose-response curves in the presence and absence of test compounds were constructed using the 4-parameter logistic equation, y=yₘₐₓ+((yₘᵢₙ−yₘₐₓ)/1+(x/C)^{D}), where C=EC₅₀ and D=slope factor.

The potency of PAM in GTPγS assays was determined by plotting the log EC₅₀ for GABA against the log concentration of the positive allosteric modulator in the presence of which the measurement was performed.

Generally, the potency of the compounds of formula (I) ranges from EC₅₀s between 20 μM and 0.001 μM. Examples of individual EC₅₀ values:

| Compound | EC₅₀ (μM) |
|---|---|
| Example 1 | 6.33 |
| Example 2 | 1.5 |
| Example 4 | 1.6 |
| Example 6 | 16.4 |
| Example 7 | 7.9 |
| Example 8 | 2.7 |
| Example 9 | 2.1 |
| Example 10 | 14.6 |
| Example 11 | 8.5 |
| Example 12 | 6 |
| Example 13 | 5.2 |
| Example 14 | 12.3 |
| Example 15 | 7.8 |
| Example 16 | 6.8 |
| Example 18 | 3.3 |
| Example 19 | 3.7 |
| Example 20 | 3 |
| Example 21 | 7.2 |
| Example 22 | 2.5 |
| Example 23 | 2.1 |
| Example 24 | 5.1 |
| Example 25 | 5.3 |
| Example 26 | 3.5 |
| Example 27 | 15.6 |
| Example 28 | 2.6 |
| Example 29 | 8.6 |
| Example 30 | 4.6 |
| Example 31 | 5.5 |
| Example 32 | 7.9 |
| Example 33 | 3.7 |
| Example 34 | 11.3 |
| Example 35 | 5.6 |
| Example 36 | 6.6 |
| Example 37 | 1.7 |
| Example 38 | 6.8 |
| Example 39 | 1.8 |
| Example 40 | 0.6 |
| Example 41 | 2 |
| Example 42 | 11.3 |
| Example 43 | 2.7 |
| Example 44 | 12.5 |
| Example 46 | 16.9 |
| Example 47 | 8.7 |
| Example 48 | 12.1 |
| Example 49 | 13.4 |
| Example 50 | 10 |
| Example 51 | 11.9 |
| Example 52 | 8.8 |
| Example 54 | 5.89 |
| Example 56 | 15 |
| Example 57 | 2.6 |
| Example 58 | 2.7 |
| Example 59 | 5.9 |
| Example 60 | 17.2 |
| Example 61 | 4.2 |
| Example 62 | 5.7 |
| Example 63 | 13.8 |
| Example 64 | 11.1 |
| Example 66 | 17.3 |
| Example 67 | 15.1 |
| Example 68 | 15.7 |
| Example 69 | 19.7 |
| Example 70 | 6.1 |
| Example 71 | 11.3 |
| Example 72 | 8.93 |
| Example 74 | 9.9 |
| Example 75 | 3.5 |
| Example 76 | 4.9 |
| Example 77 | 6.64 |
| Example 78 | 4.5 |
| Example 79 | 14.53 |
| Example 80 | 5.2 |
| Example 81 | 11.5 |
| Example 82 | 5.6 |
| Example 83 | 7.8 |
| Example 84 | 5.1 |
| Example 85 | 1.9 |
| Example 86 | 3.6 |
| Example 87 | 15.6 |
| Example 88 | 3.7 |
| Example 89 | 1.8 |
| Example 91 | 8.3 |
| Example 92 | 7.1 |
| Example 93 | 7.2 |
| Example 94 | 9 |
| Example 95 | 4.5 |
| Example 96 | 12.2 |

### Effect of Compounds in IBS Model (Colorectal Distension)

### Colorectal Distension (CRD)

For CRD, a 3 cm polyethylene balloon with a connecting catheter (made in-house) was inserted in the distal colon, 2 cm from the base of the balloon to the anus, during light isoflurane anaesthesia (Forene®, Abbott Scandinavia AB, Sweden). The catheter was fixed to the base of the tail with tape. At the same time, an intravenous catheter (Neoflon®, Becton Dickinson AB, Sweden) was inserted in a tail vein for compounds administration. Thereafter, rats were placed in Bollman cages and allowed to recover from sedation for at least 15 min before starting the experiments.

During the CRD procedure, the balloons were connected to pressure transducers (P-602, CFM-k33, 100 mmHg; Bronkhorst Hi-Tec, Veenendal, The Netherlands). A customized barostat (AstraZeneca, Mölndal, Sweden) was used to control the air inflation and intraballoon pressure. A customized computer software (PharmLab on-line 4.0.1) running on a standard PC was used to control the barostat and to perform data collection and storage. The distension paradigm generated by the barostat were achieved by generating pulse patterns on an analog output channel. The CRD paradigms used consisted on repeated phasic distensions, 12 times at 80 mmHg, with a pulse duration of 30 s at 5 min intervals.

Responses to CRD were assessed by recording and quantitation of phasic changes in intraballoon pressure during the distending pulses. Pressue oscillations during the isobaric inflation of the intracolonic balloon reflect abdominal muscle contractions associated to the distension procedure and, therefore, are considered a valid assessment of the visceromotor response (VMR) associated to the presence of pain of visceral origin.

### Data Collection and Analysis

The balloon pressure signals were sampled at 50 Hz and afterwards subjected to digital filtering. A highpass filter at 1 Hz was used to separate the contraction-induced pressure changes from the slow varying pressure generated by the barostat. A resistance in the airflow between the pressure generator and the pressure transducer further enhanced the pressure variations induced by abdominal contractions of the animal. In addition, a band-stop filtere at 49-51 Hz was used to remove line frequency interference. A customized computer software (PharmLab off-line 4.0.1) was used to quantify the phasic changes of the balloon pressure signals. The average rectified value (ARV) of the balloon pressure signals was calculated for the 30 s period before the pulse (baseline activity) and for the duration of the pulse (as a measure of the VMR to distension). When performing pulses analysis, the first and last second of each pulse were excluded since they reflect artefact signals produced by the barostat during inflation and deflation of the balloon and do not originate from the animal.

### Results

The effect of the positive allosteric modulators was examined on the VMR to isobaric CRD in rats. A paradigm consisting of 12 distensions at 80 mmHg was used. The compounds were administered at a dose of 1 to 50 μmol/kg and VMR responses to CRD compared to the vehicle control. The compounds were effective reducing the VMR to CRD (at least a 20% inhibition compared to the vehicle used).

1. A compound of the general formula (I) 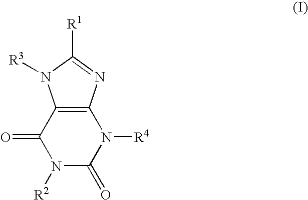
or a pharmaceutically acceptable salt thereof;
wherein
R¹ is selected from halogen; C₁-C₁₀ alkyl; C₁-C₁₀ alkoxy; hydroxy-C₁-C₁₀ alkyl; C₁-C₁₀ alkoxy-C₁-C₁₀ alkyl; C₃-C₁₀ cycloalkyl; amino substituted by one or more of C₁-C₁₀ alkyl and C₁-C₁₀ alkoxy-C₁-C₁₀ alkyl; and heterocyclyl unsubstituted or substituted by one or more of C₁-C₁₀ alkyl, C₁-C₁₀ alkoxy, C₁-C₁₀ alkoxy-C₁-C₁₀ alkyl, di-C₁-C₁₀ alkylamino, oxo and heterocyclyl-C₁-C₁₀ alkyl;
R² is selected from benzyl substituted by one or more of halogen; cyano; C₁-C₁₀ alkyl; C₁-C₁₀ alkoxy; aroyl; halo-C₁-C₁₀ alkyl; aryl-C₁-C₁₀ alkoxy and C₁-C₁₀ alkoxycarbonyl; 2-naphthylmethyl; 1-(4-chlorophenyl)-5-(trifluoromethyl)-1H-pyrazol-4-ylmethyl; 2-(4-chlorophenyl)ethyl; 2,1,3-benzothiadiazol-5-ylmethyl; and 1-[5-(trifluoromethyl)]-1,3-benzothiazol-2-ylmethyl;
R³ is selected from C₁-C₁₀ alkyl and aryl substituted by one or more of halogen;
R⁴ is selected from ethyl; isobutyl; propyl; 3,3-dimethylbutyl; C₁-C₁₀ alkyl substituted by one or more of hydroxy, oxo, C₁-C₁₀ alkoxy, C₁-C₁₀ alkoxycarbonylamino, tri-C₁-C₁₀ alkylsilyl, tri-C₁-C₁₀ alkylsilyloxy, C₁-C₁₀ alkylsulfonyl and aryloxy, wherein the aryloxy is substituted by one or more of halo-C₁-C₁₀ alkyl; amino-C₁-C₁₀ alkyl substituted by oxo; di-C₁-C₁₀ alkylamino-C₁-C₁₀ alkyl unsubstituted or substituted by one or more of oxo; halo-C₁-C₁₀ alkyl unsubstituted or substituted by one or more of hydroxy; C₁-C₁₀ alkoxycarbonyl-C₁-C₁₀ alkyl; C₂-C₁₀ alkenyl; C₃-C₁₀ cycloalkyl-C₁-C₁₀ alkyl unsubstituted or substituted by oxo; aryl-C₁-C₁₀ alkyl unsubstituted or substituted by one or more of halogen, C₁-C₁₀ alkoxy, halo-C₁-C₁₀ alkyl, halo-C₁-C₁₀ alkoxy, halo-C₁-C₁₀ alkylthio, C₁-C₁₀ alkylsulfonyl, oxo and heteroaryl; heteroaryl-C₁-C₁₀ alkyl unsubstituted or substituted by one or more of halogen, C₁-C₁₀ alkyl, C₁-C₁₀ alkylsulfonyl, halo-C₁-C₁₀ alkyl, oxo and aryl, wherein the aryl group is unsubstituted or substituted by halogen; heterocyclyl-C₁-C₁₀ alkyl unsubstituted or substituted by one or more of halogen, oxo and aryl;
with the proviso that the compound is not:
1-benzyl-3-isobutylxanthine;
1-benzyl-3-butylxanthine;
1-(4-chlorobenzyl)-3-ethyl-8-isopropylxanthine;
1,3-dibenzylxanthine; and
1,3-di-(4-chlorobenzyl)-8-isopropylxanthine.

2. The compound according to claim 1, wherein
R¹ is selected from bromo; methyl; ethyl; tert-butyl; methoxy; 1-hydroxyethyl; methoxymethyl; cyclobutyl; cyclopentyl; cyclohexyl; amino substituted by one or more of methyl, etyl and 2-methoxyethyl; azetidin-1-yl; morpholin-4-yl; piperazin-1-yl substituted by one or more of methyl; piperidin-1-yl unsubstituted or substituted by one or more of methoxy; pyrrolidin-1-yl unsubstituted or substituted by one or more of methoxymethyl, dimethylamino, oxo and pyrrolidin-1-ylmethyl; tetrahydrofuran-3-yl; and thiomorpholin-4-yl;
R² is selected from benzyl substituted by one or more of bromo, chloro, fluoro, cyano, isopropyl, methoxy, benzoyl, trifluoromethyl, benzyloxy and carbomethoxy; 2-naphthylmethyl; 1-(4-chlorophenyl)-5-(trifluoromethyl)-1H-pyrazol-4-ylmethyl; 2-(4-chlorophenyl)ethyl; 2,1,3-benzothiadiazol-5-ylmethyl; and 1-[5-(trifluoromethyl)]-1,3-benzothiazol-2-ylmethyl;
R³ is selected from methyl; ethyl; isopropyl; and 4-fluorophenyl;
R⁴ is selected from ethyl; isobutyl; propyl; 3,3-dimethylbutyl; 3-hydroxypropyl; 2,3-dihydroxypropyl; 2-oxobutyl; 3,3-dimethyl-2-oxobutyl; 2-methoxyethyl; 2,2-dimethoxyethyl; 3-tert-butoxypropyl; 2-tert-butoxy-2-oxoethyl; 2-tert-butoxycarbonylaminoethyl; 2-(trimethylsilyl)ethyl; trimethylsilylmethyl; 2-tert-butyl(dimethyl)silyloxyethyl; 3-(tert-butylsulfonyl)propyl; 3-[4-(trifluoromethyl)phenoxy]propyl; 2-amino-2-oxoethyl; 2-diethylaminoethyl; 2-diisopropylamino-2-oxoethyl; 3,3,3-trifluoropropyl; 4,4,4-trifluorobutyl; 3,3,3-trifluoro-2-hydroxypropyl; carbomethoxymethyl; allyl; cyclohexylmethyl; 4-cyclohexylbutyl; 2-[(3S,5S,7S)-adamantan-1-yl]-2-oxoethyl; benzyl unsubstituted or substituted by one or more of chloro, methoxy, trifluoromethyl, difluoromethoxy, trifluoromethylthio, methylsulfonyl and 1H-pyrazol-1-yl; 2-oxo-2-phenylethyl; 3-chloro-4-isopropylsulfonyl-2-thienylmethyl; 1-(4-chlorophenyl)-5-(trifluoromethyl)-1H-pyrazol-4-ylmethyl; 3-(1H-imidazol-1-yl)propyl; 5-methylisoxazol-3-ylmethyl; 5-methyl-3-phenylisoxazol-4-ylmethyl; 2-oxo-2-pyridin-4-ylethyl; 2-(1H-pyrrol-1-yl)ethyl; pyridin-2-ylmethyl; pyridin-3-ylmethyl; 2-(3,3-difluoropyrrolidin-1-yl)-2-oxoethyl; 2,3-dihydro-1,4-benzodioxin-2-ylmethyl; 3-(1,4-dioxa-8-azaspiro[4.5]dec-8-yl)propyl; 1,3-dioxolan-2-ylmethyl; (2R)-5-oxopyrrolidin-2-ylmethyl; (2S)-5-oxopyrrolidin-2-ylmethyl; 3-(4-phenylpiperazin-1-yl)propyl; and 3-pyrrolidin-1-ylpropyl.

3. The compound according to claim 1, which is selected from:
3-benzyl-1-(4-chlorobenzyl)-8-ethyl-7-methyl-3,7-dihydro-1H-purine-2,6-dione;
1-(4-chlorobenzyl)-3-(3,3-dimethylbutyl)-8-ethyl-7-methyl-3,7-dihydro-1H-purine-2,6-dione;
1-(4-chlorobenzyl)-3-(3,3-dimethyl-2-oxobutyl)-8-ethyl-7-methyl-3,7-dihydro-1H-purine-2,6-dione;
1-(4-chlorobenzyl)-8-ethyl-7-methyl-3-{[(2R)-5-oxopyrrolidin-2-yl]methyl}-3,7-dihydro-1H-purine-2,6-dione;
1-(4-chlorobenzyl)-8-ethyl-7-methyl-3-(2-oxo-2-pyridin-4-ylethyl)-3,7-dihydro-1H-purine-2,6-dione;
1-(4-chlorobenzyl)-8-ethyl-3-isobutyl-7-methyl-3,7-dihydro-1H-purine-2,6-dione;
1-(4-chlorobenzyl)-8-ethyl-7-methyl-3-[(trimethylsilyl)methyl]-3,7-dihydro-1H-purine-2,6-dione;
1-(4-chlorobenzyl)-8-ethyl-7-methyl-3-{[(2S)-5-oxopyrrolidin-2-yl]methyl}-3,7-dihydro-1H-purine-2,6-dione;
methyl [1-(4-chlorobenzyl)-8-ethyl-7-methyl-2,6-dioxo-1,2,6,7-tetrahydro-3H-purin-3-yl]acetate;
3-allyl-1-(4-chlorobenzyl)-8-ethyl-7-methyl-3,7-dihydro-1H-purine-2,6-dione;
1,3-bis(4-chlorobenzyl)-8-ethyl-7-methyl-3,7-dihydro-1H-purine-2,6-dione;
1-(4-chlorobenzyl)-3-(1,3-dioxolan-2-ylmethyl)-8-ethyl-7-methyl-3,7-dihydro-1H-purine-2,6-dione;
1-(4-chlorobenzyl)-8-ethyl-7-methyl-3-(pyridin-2-ylmethyl)-3,7-dihydro-1H-purine-2,6-dione;
1-(4-chlorobenzyl)-8-ethyl-7-methyl-3-[(5-methylisoxazol-3-yl)methyl]-3,7-dihydro-1H-purine-2,6-dione;
1-(4-chlorobenzyl)-8-ethyl-7-methyl-3-(pyridin-3-ylmethyl)-3,7-dihydro-1H-purine-2,6-dione;
1-(4-chlorobenzyl)-3-[4-(difluoromethoxy)benzyl]-8-ethyl-7-methyl-3,7-dihydro-1H-purine-2,6-dione;
1-(4-chlorobenzyl)-3-(cyclohexylmethyl)-8-ethyl-7-methyl-3,7-dihydro-1H-purine-2,6-dione;
3-(3-tert-butoxypropyl)-1-(4-chlorobenzyl)-8-ethyl-7-methyl-3,7-dihydro-1H-purine-2,6-dione;
1-(4-chlorobenzyl)-8-ethyl-7-methyl-3-[4-(methylsulfonyl)benzyl]-3,7-dihydro-1H-purine-2,6-dione;
1-(4-chlorobenzyl)-8-ethyl-7-methyl-3-(3,3,3-trifluoro-2-hydroxypropyl)-3,7-dihydro-1H-purine-2,6-dione;
1-(4-chlorobenzyl)-3-(2,3-dihydro-1,4-benzodioxin-2-ylmethyl)-8-ethyl-7-methyl-3,7-dihydro-1H-purine-2,6-dione;
1-(4-chlorobenzyl)-8-ethyl-7-methyl-3-{4-[(trifluoromethyl)thio]benzyl}-3,7-dihydro-1H-purine-2,6-dione;
1-(4-chlorobenzyl)-8-ethyl-7-methyl-3-[4-(1H-pyrazol-1-yl)benzyl]-3,7-dihydro-1H-purine-2,6-dione;
1-(4-chlorobenzyl)-3-[2-(diethylamino)ethyl]-8-ethyl-7-methyl-3,7-dihydro-1H-purine-2,6-dione;
1-(4-chlorobenzyl)-8-ethyl-7-methyl-3-(2-oxo-2-phenylethyl)-3,7-dihydro-1H-purine-2,6-dione;
3-(2-{[tert-butyl(dimethyl)silyl]oxy}ethyl)-1-(4-chlorobenzyl)-8-ethyl-7-methyl-3,7-dihydro-1H-purine-2,6-dione;
1-(4-chlorobenzyl)-8-ethyl-7-methyl-3-[(5-methyl-3-phenylisoxazol-4-yl)methyl]-3,7-dihydro-1H-purine-2,6-dione;
1-(4-chlorobenzyl)-8-ethyl-7-methyl-3-[4-(trifluoromethyl)benzyl]-3,7-dihydro-1H-purine-2,6-dione;
1-(4-chlorobenzyl)-8-ethyl-3-(2-methoxyethyl)-7-methyl-3,7-dihydro-1H-purine-2,6-dione;
1-(4-chlorobenzyl)-8-ethyl-7-methyl-3-(2-oxobutyl)-3,7-dihydro-1H-purine-2,6-dione;
3-[3-(tert-butylsulfonyl)propyl]-1-(4-chlorobenzyl)-8-ethyl-7-methyl-3,7-dihydro-1H-purine-2,6-dione;
tert-butyl [1-(4-chlorobenzyl)-8-ethyl-7-methyl-2,6-dioxo-1,2,6,7-tetrahydro-3H-purin-3-yl]acetate;
1-(4-chlorobenzyl)-8-ethyl-7-methyl-3-{3-[4-(trifluoromethyl)phenoxy]propyl}-3,7-dihydro-1H-purine-2,6-dione;
1-(4-chlorobenzyl)-8-ethyl-7-methyl-3-[2-(1H-pyrrol-1-yl)ethyl]-3,7-dihydro-1H-purine-2,6-dione;
1-(4-chlorobenzyl)-8-ethyl-3-(3-hydroxypropyl)-7-methyl-3,7-dihydro-1H-purine-2,6-dione;
1-(4-chlorobenzyl)-3-{[3-chloro-4-(isopropylsulfonyl)-2-thienyl]methyl}-8-ethyl-7-methyl-3,7-dihydro-1H-purine-2,6-dione;
1-(3,4-dichlorobenzyl)-3-(3,3-dimethyl-2-oxobutyl)-8-ethyl-7-methyl-3,7-dihydro-1H-purine-2,6-dione;
1-(3,4-dichlorobenzyl)-3-(3,3-dimethylbutyl)-8-ethyl-7-methyl-3,7-dihydro-1H-purine-2,6-dione;
3-{2-[(3S,5S,7S)-adamantan-1-yl]-2-oxoethyl}-(3,4-dichlorobenzyl)-8-ethyl-7-methyl-3,7-dihydro-1H-purine-2,6-dione;
1-(3,4-dichlorobenzyl)-8-ethyl-7-methyl-3-[2-(trimethylsilyl)ethyl]-3,7-dihydro-1H-purine-2,6-dione;
3-(4-cyclohexylbutyl)-1-(3,4-dichlorobenzyl)-8-ethyl-7-methyl-3,7-dihydro-1H-purine-2,6-dione;
1-(4-chlorobenzyl)-8-ethyl-7-methyl-3-(3-pyrrolidin-1-ylpropyl)-3,7-dihydro-1H-purine-2,6-dione;
1-(4-chlorobenzyl)-8-ethyl-7-methyl-3-[3-(4-phenylpiperazin-1-yl)propyl]-3,7-dihydro-1H-purine-2,6-dione;
1-(4-chlorobenzyl)-3-[3-(1,4-dioxa-8-azaspiro[4.5]dec-8-yl)propyl]-8-ethyl-7-methyl-3,7-dihydro-1H-purine-2,6-dione;
1-(4-chlorobenzyl)-8-ethyl-3-[3-(1H-imidazol-1-yl)propyl]-7-methyl-3,7-dihydro-1H-purine-2,6-dione;
1-(4-chlorobenzyl)-3-[2-(3,3-difluoropyrrolidin-1-yl)-2-oxoethyl]-8-ethyl-7-methyl-3,7-dihydro-1H-purine-2,6-dione;
2-[1-(4-chlorobenzyl)-8-ethyl-7-methyl-2,6-dioxo-1,2,6,7-tetrahydro-3H-purin-3-yl]-N,N-diisopropylacetamide;
1-(4-chlorobenzyl)-3-(2,2-dimethoxyethyl)-8-ethyl-7-methyl-3,7-dihydro-1H-purine-2,6-dione;
1-(4-chlorobenzyl)-8-ethyl-7-methyl-3-propyl-3,7-dihydro-1H-purine-2,6-dione;
1-[4-(benzyloxy)benzyl]-8-ethyl-7-methyl-3-propyl-3,7-dihydro-1H-purine-2,6-dione;
1-(3,4-dichlorobenzyl)-8-ethyl-7-methyl-3-propyl-3,7-dihydro-1H-purine-2,6-dione;
8-ethyl-7-methyl-1-(2-naphthylmethyl)-3-propyl-3,7-dihydro-1H-purine-2,6-dione;
1-{[1-(4-chlorophenyl)-5-(trifluoromethyl)-1H-pyrazol-4-yl]methyl}-8-ethyl-7-methyl-3-propyl-3,7-dihydro-1H-purine-2,6-dione;
1-(2,4-dichlorobenzyl)-8-ethyl-7-methyl-3-propyl-3,7-dihydro-1H-purine-2,6-dione;
1-(4-bromobenzyl)-8-ethyl-7-methyl-3-propyl-3,7-dihydro-1H-purine-2,6-dione;
8-ethyl-7-methyl-3-propyl-1-[4-(trifluoromethyl)benzyl]-3,7-dihydro-1H-purine-2,6-dione;
1-[2-(4-chlorophenyl)ethyl]-8-ethyl-7-methyl-3-propyl-3,7-dihydro-1H-purine-2,6-dione;
1-(2,1,3-benzothiadiazol-5-ylmethyl)-8-ethyl-7-methyl-3-propyl-3,7-dihydro-1H-purine-2,6-dione;
8-ethyl-7-methyl-3-propyl-1-{[5-(trifluoromethyl)-1,3-benzothiazol-2-yl]methyl}-3,7-dihydro-1H-purine-2,6-dione;
1-(3-chlorobenzyl)-8-ethyl-7-methyl-3-propyl-3,7-dihydro-1H-purine-2,6-dione;
1-(4-benzoylbenzyl)-8-ethyl-7-methyl-3-propyl-3,7-dihydro-1H-purine-2,6-dione;
8-ethyl-1-(4-methoxybenzyl)-7-methyl-3-propyl-3,7-dihydro-1H-purine-2,6-dione;
8-ethyl-1-(4-isopropylbenzyl)-7-methyl-3-propyl-3,7-dihydro-1H-purine-2,6-dione;
1-(4-chlorobenzyl)-3-(2,4-dimethoxybenzyl)-8-ethyl-7-methyl-3,7-dihydro-1H-purine-2,6-dione;
1-(4-chlorobenzyl)-7,8-diethyl-3-propyl-3,7-dihydro-1H-purine-2,6-dione;
1-(4-chlorobenzyl)-8-ethyl-7-(4-fluorophenyl)-3-propyl-3,7-dihydro-1H-purine-2,6-dione;
8-methoxy-7-methyl-3-(3,3,3-trifluoropropyl)-3,7-dihydro-1H-purine-2,6-dione;
8-methoxy-7-methyl-3-(4,4,4-trifluorobutyl)-3,7-dihydro-1H-purine-2,6-dione;
1-(4-chlorobenzyl)-3-ethyl-7-(4-fluorophenyl)-8-methoxy-3,7-dihydro-1H-purine-2,6-dione;
1-(4-chlorobenzyl)-8-methoxy-7-methyl-3-propyl-3,7-dihydro-1H-purine-2,6-dione;
1-(4-fluorobenzyl)-8-methoxy-7-methyl-3-propyl-3,7-dihydro-1H-purine-2,6-dione;
1-(4-chlorobenzyl)-8-methoxy-7-methyl-3-(3,3,3-trifluoropropyl)-3,7-dihydro-1H-purine-2,6-dione;
1-(4-chlorobenzyl)-8-methoxy-7-methyl-3-(4,4,4-trifluorobutyl)-3,7-dihydro-1H-purine-2,6-dione;
1-(4-chlorobenzyl)-8-(dimethylamino)-7-methyl-3-propyl-3,7-dihydro-1H-purine-2,6-dione;
8-azetidin-1-yl-1-(4-chlorobenzyl)-7-methyl-3-propyl-3,7-dihydro-1H-purine-2,6-dione;
1-(4-chlorobenzyl)-8-(4-methoxypiperidin-1-yl)-7-methyl-3-propyl-3,7-dihydro-1H-purine-2,6-dione;
1-(4-chlorobenzyl)-7-methyl-8-piperidin-1-yl-3-propyl-3,7-dihydro-1H-purine-2,6-dione;
1-(4-chlorobenzyl)-7-methyl-3-propyl-8-pyrrolidin-1-yl-3,7-dihydro-1H-purine-2,6-dione;
1-(4-chlorobenzyl)-7-methyl-8-(4-methylpiperazin-1-yl)-3-propyl-3,7-dihydro-1H-purine-2,6-dione;
1-(4-chlorobenzyl)-7-methyl-3-propyl-8-thiomorpholin-4-yl-3,7-dihydro-1H-purine-2,6-dione;
1-(4-chlorobenzyl)-8-(diethylamino)-7-methyl-3-propyl-3,7-dihydro-1H-purine-2,6-dione;
1-(4-chlorobenzyl)-8-[(2-methoxyethyl)(methyl)amino]-7-methyl-3-propyl-3,7-dihydro-1H-purine-2,6-dione;
1-(4-chlorobenzyl)-7-methyl-8-morpholin-4-yl-3-propyl-3,7-dihydro-1H-purine-2,6-dione;
1-(4-chlorobenzyl)-8-[(2S)-2-(methoxymethyl)pyrrolidin-1-yl]-7-methyl-3-propyl-3,7-dihydro-1H-purine-2,6-dione;
1-(4-chlorobenzyl)-7-methyl-3-propyl-8-[(2S)-2-(pyrrolidin-1-ylmethyl)pyrrolidin-1-yl]-3,7-dihydro-1H-purine-2,6-dione;
8-bromo-1-(4-chlorobenzyl)-7-methyl-3-propyl-3,7-dihydro-1H-purine-2,6-dione; and
1-(4-chlorobenzyl)-8-(1-hydroxyethyl)-7-methyl-3-propyl-3,7-dihydro-1H-purine-2,6-dione;
or a pharmaceutically acceptable salt thereof.

4-5. (canceled)

6. A pharmaceutical composition comprising a compound according to claim 1 as an active ingredient and a pharmaceutically acceptable carrier or diluent.

7-15. (canceled)

16. A method of treating gastroesophageal reflux disease (GERD) comprising administering an effective amount of a compound of formula (I) or a pharmaceutically acceptable salt thereof, optionally in combination with a GABA_{B} receptor agonist, to a subject, wherein the compound of formula (I) comprises 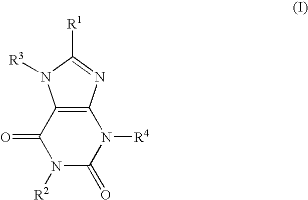
wherein
R¹ is selected from halogen; C₁-C₁₀ alkyl; C₁-C₁₀ alkoxy; hydroxy-C₁-C₁₀ alkyl; C₁-C₁₀ alkoxy-C₁-C₁₀ alkyl; C₃-C₁₀ cycloalkyl; amino substituted by one or more of C₁-C₁₀ alkyl and C₁-C₁₀ alkoxy-C₁-C₁₀ alkyl; and heterocyclyl unsubstituted or substituted by one or more of C₁-C₁₀ alkyl, C₁-C₁₀ alkoxy, C₁-C₁₀ alkoxy-C₁-C₁₀ alkyl, di-C₁-C₁₀ alkylamino, oxo and heterocyclyl-C₁-C₁₀ alkyl;
R² is selected from benzyl substituted by one or more of halogen; cyano; C₁-C₁₀ alkyl; C₁-C₁₀ alkoxy; aroyl; halo-C₁-C₁₀ alkyl; aryl-C₁-C₁₀ alkoxy and C₁-C₁₀ alkoxycarbonyl; 2-naphthylmethyl; 1-(4-chlorophenyl)-5-(trifluoromethyl)-1H-pyrazol-4-ylmethyl; 2-(4-chlorophenyl)ethyl; 2,1,3-benzothiadiazol-5-ylmethyl; and 1-[5-(trifluoromethyl)]-1,3-benzothiazol-2-ylmethyl;
R³ is selected from C₁-C₁₀ alkyl and aryl substituted by one or more of halogen; and
R⁴ is selected from ethyl; isobutyl; propyl; 3,3-dimethylbutyl; C₁-C₁₀ alkyl substituted by one or more of hydroxy, oxo, C₁-C₁₀ alkoxy, C₁-C₁₀ alkoxycarbonylamino, tri-C₁-C₁₀ alkylsilyl, tri-C₁-C₁₀ alkylsilyloxy, C₁-C₁₀ alkylsulfonyl and aryloxy, wherein the aryloxy is substituted by one or more of halo-C₁-C₁₀ alkyl; amino-C₁-C₁₀ alkyl substituted by oxo; di-C₁-C₁₀ alkylamino-C₁-C₁₀ alkyl unsubstituted or substituted by one or more of oxo; halo-C₁-C₁₀ alkyl unsubstituted or substituted by one or more of hydroxy; C₁-C₁₀ alkoxycarbonyl-C₁-C₁₀ alkyl; C₂-C₁₀ alkenyl; C₃-C₁₀ cycloalkyl-C₁-C₁₀ alkyl unsubstituted or substituted by oxo; aryl-C₁-C₁₀ alkyl unsubstituted or substituted by one or more of halogen, C₁-C₁₀ alkoxy, halo-C₁-C₁₀ alkyl, halo-C₁-C₁₀ alkoxy, halo-C₁-C₁₀ alkylthio, C₁-C₁₀ alkylsulfonyl, oxo and heteroaryl; heteroaryl-C₁-C₁₀ alkyl unsubstituted or substituted by one or more of halogen, C₁-C₁₀ alkyl, C₁-C₁₀ alkylsulfonyl, halo-C₁-C₁₀ alkyl, oxo and aryl, wherein the aryl group is unsubstituted or substituted by halogen; heterocyclyl-C₁-C₁₀ alkyl unsubstituted or substituted by one or more of halogen, oxo and aryl.

17. A method for the prevention of reflux comprising administering an effective amount of a compound of formula (I) or a pharmaceutically acceptable salt thereof, optionally in combination with a GABA_{B} receptor agonist, to a subject, wherein the compound of formula (I) comprises 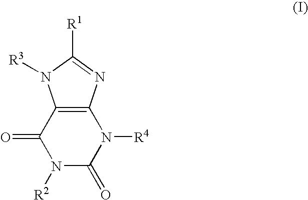
wherein
R¹ is selected from halogen; C₁-C₁₀ alkyl; C₁-C₁₀ alkoxy; hydroxy-C₁-C₁₀ alkyl; C₁-C₁₀ alkoxy-C₁-C₁₀ alkyl; C₃-C₁₀ cycloalkyl; amino substituted by one or more of C₁-C₁₀ alkyl and C₁-C₁₀ alkoxy-C₁-C₁₀ alkyl; and heterocyclyl unsubstituted or substituted by one or more of C₁-C₁₀ alkyl, C₁-C₁₀ alkoxy, C₁-C₁₀ alkoxy-C₁-C₁₀ alkyl, di-C₁-C₁₀ alkylamino, oxo and heterocyclyl-C₁-C₁₀ alkyl;
R² is selected from benzyl substituted by one or more of halogen; cyano; C₁-C₁₀ alkyl; C₁-C₁₀ alkoxy; aroyl; halo-C₁-C₁₀ alkyl; aryl-C₁-C₁₀ alkoxy and C₁-C₁₀ alkoxycarbonyl; 2-naphthylmethyl; 1-(4-chlorophenyl)-5-(trifluoromethyl)-1H-pyrazol-4-ylmethyl; 2-(4-chlorophenyl)ethyl; 2,1,3-benzothiadiazol-5-ylmethyl; and 1-[5-(trifluoromethyl)]-1,3-benzothiazol-2-ylmethyl;
R³ is selected from C₁-C₁₀ alkyl and aryl substituted by one or more of halogen; and
R⁴ is selected from ethyl; isobutyl; propyl; 3,3-dimethylbutyl; C₁-C₁₀ alkyl substituted by one or more of hydroxy, oxo, C₁-C₁₀ alkoxy, C₁-C₁₀ alkoxycarbonylamino, tri-C₁-C₁₀ alkylsilyl, tri-C₁-C₁₀ alkylsilyloxy, C₁-C₁₀ alkylsulfonyl and aryloxy, wherein the aryloxy is substituted by one or more of halo-C₁-C₁₀ alkyl; amino-C₁-C₁₀ alkyl substituted by oxo; di-C₁-C₁₀ alkylamino-C₁-C₁₀ alkyl unsubstituted or substituted by one or more of oxo; halo-C₁-C₁₀ alkyl unsubstituted or substituted by one or more of hydroxy; C₁-C₁₀ alkoxycarbonyl-C₁-C₁₀ alkyl; C₂-C₁₀ alkenyl; C₃-C₁₀ cycloalkyl-C₁-C₁₀ alkyl unsubstituted or substituted by oxo; aryl-C₁-C₁₀ alkyl unsubstituted or substituted by one or more of halogen, C₁-C₁₀ alkoxy, halo-C₁-C₁₀ alkyl, halo-C₁-C₁₀ alkoxy, halo-C₁-C₁₀ alkylthio, C₁-C₁₀ alkylsulfonyl, oxo and heteroaryl; heteroaryl-C₁-C₁₀ alkyl unsubstituted or substituted by one or more of halogen, C₁-C₁₀ alkyl, C₁-C₁₀ alkylsulfonyl, halo-C₁-C₁₀ alkyl, oxo and aryl, wherein the aryl group is unsubstituted or substituted by halogen; heterocyclyl-C₁-C₁₀ alkyl unsubstituted or substituted by one or more of halogen, oxo and aryl.

18. A method for the inhibition of transient lower esophageal sphincter relaxations (TLESRs) comprising administering an effective amount of a compound of formula (I) or a pharmaceutically acceptable salt thereof, optionally in combination with a GABA_{B} receptor agonist, to a subject, wherein the compound of formula (I) comprises 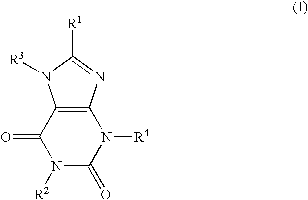
wherein
R¹ is selected from halogen; C₁-C₁₀ alkyl; C₁-C₁₀ alkoxy; hydroxy-C₁-C₁₀ alkyl; C₁-C₁₀ alkoxy-C₁-C₁₀ alkyl; C₃-C₁₀ cycloalkyl; amino substituted by one or more of C₁-C₁₀ alkyl and C₁-C₁₀ alkoxy-C₁-C₁₀ alkyl; and heterocyclyl unsubstituted or substituted by one or more of C₁-C₁₀ alkyl, C₁-C₁₀ alkoxy, C₁-C₁₀ alkoxy-C₁-C₁₀ alkyl, di-C₁-C₁₀ alkylamino, oxo and heterocyclyl-C₁-C₁₀ alkyl;
R² is selected from benzyl substituted by one or more of halogen; cyano; C₁-C₁₀ alkyl; C₁-C₁₀ alkoxy; aroyl; halo-C₁-C₁₀ alkyl; aryl-C₁-C₁₀ alkoxy and C₁-C₁₀ alkoxycarbonyl; 2-naphthylmethyl; 1-(4-chlorophenyl)-5-(trifluoromethyl)-1H-pyrazol-4-ylmethyl; 2-(4-chlorophenyl)ethyl; 2,1,3-benzothiadiazol-5-ylmethyl; and 1-[5-(trifluoromethyl)]-1,3-benzothiazol-2-ylmethyl;
R³ is selected from C₁-C₁₀ alkyl and aryl substituted by one or more of halogen; and
R⁴ is selected from ethyl; isobutyl; propyl; 3,3-dimethylbutyl; C₁-C₁₀ alkyl substituted by one or more of hydroxy, oxo, C₁-C₁₀ alkoxy, C₁-C₁₀ alkoxycarbonylamino, tri-C₁-C₁₀ alkylsilyl, tri-C₁-C₁₀ alkylsilyloxy, C₁-C₁₀ alkylsulfonyl and aryloxy, wherein the aryloxy is substituted by one or more of halo-C₁-C₁₀ alkyl; amino-C₁-C₁₀ alkyl substituted by oxo; di-C₁-C₁₀ alkylamino-C₁-C₁₀ alkyl unsubstituted or substituted by one or more of oxo; halo-C₁-C₁₀ alkyl unsubstituted or substituted by one or more of hydroxy; C₁-C₁₀ alkoxycarbonyl-C₁-C₁₀ alkyl; C₂-C₁₀ alkenyl; C₃-C₁₀ cycloalkyl-C₁-C₁₀ alkyl unsubstituted or substituted by oxo; aryl-C₁-C₁₀ alkyl unsubstituted or substituted by one or more of halogen, C₁-C₁₀ alkoxy, halo-C₁-C₁₀ alkyl, halo-C₁-C₁₀ alkoxy, halo-C₁-C₁₀ alkylthio, C₁-C₁₀ alkylsulfonyl, oxo and heteroaryl; heteroaryl-C₁-C₁₀ alkyl unsubstituted or substituted by one or more of halogen, C₁-C₁₀ alkyl, C₁-C₁₀ alkylsulfonyl, halo-C₁-C₁₀ alkyl, oxo and aryl, wherein the aryl group is unsubstituted or substituted by halogen; heterocyclyl-C₁-C₁₀ alkyl unsubstituted or substituted by one or more of halogen, oxo and aryl.

19. A method for the treatment of a functional gastrointestinal disorder comprising administering an effective amount of a compound of formula (I) or a pharmaceutically acceptable salt thereof, optionally in combination with a GABA_{B} receptor agonist, to a subject, wherein the compound of formula (I) comprises 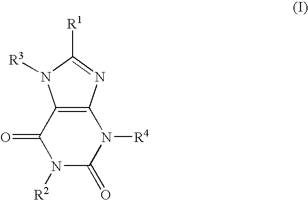
wherein
R¹ is selected from halogen; C₁-C₁₀ alkyl; C₁-C₁₀ alkoxy; hydroxy-C₁-C₁₀ alkyl; C₁-C₁₀ alkoxy-C₁-C₁₀ alkyl; C₃-C₁₀ cycloalkyl; amino substituted by one or more of C₁-C₁₀ alkyl and
C₁-C₁₀ alkoxy-C₁-C₁₀ alkyl; and heterocyclyl unsubstituted or substituted by one or more of C₁-C₁₀ alkyl, C₁-C₁₀ alkoxy, C₁-C₁₀ alkoxy-C₁-C₁₀ alkyl, di-C₁-C₁₀ alkylamino, oxo and heterocyclyl-C₁-C₁₀ alkyl;
R² is selected from benzyl substituted by one or more of halogen; cyano; C₁-C₁₀ alkyl; C₁-C₁₀ alkoxy; aroyl; halo-C₁-C₁₀ alkyl; aryl-C₁-C₁₀ alkoxy and C₁-C₁₀ alkoxycarbonyl; 2-naphthylmethyl; 1-(4-chlorophenyl)-5-(trifluoromethyl)-1H-pyrazol-4-ylmethyl; 2-(4-chlorophenyl)ethyl; 2,1,3-benzothiadiazol-5-ylmethyl; and 1-[5-(trifluoromethyl)]-1,3-benzothiazol-2-ylmethyl;
R³ is selected from C₁-C₁₀ alkyl and aryl substituted by one or more of halogen; and
R⁴ is selected from ethyl; isobutyl; propyl; 3,3-dimethylbutyl; C₁-C₁₀ alkyl substituted by one or more of hydroxy, oxo, C₁-C₁₀ alkoxy, C₁-C₁₀ alkoxycarbonylamino, tri-C₁-C₁₀ alkylsilyl, tri-C₁-C₁₀ alkylsilyloxy, C₁-C₁₀ alkylsulfonyl and aryloxy, wherein the aryloxy is substituted by one or more of halo-C₁-C₁₀ alkyl; amino-C₁-C₁₀ alkyl substituted by oxo; di-C₁-C₁₀ alkylamino-C₁-C₁₀ alkyl unsubstituted or substituted by one or more of oxo; halo-C₁-C₁₀ alkyl unsubstituted or substituted by one or more of hydroxy; C₁-C₁₀ alkoxycarbonyl-C₁-C₁₀ alkyl; C₂-C₁₀ alkenyl; C₃-C₁₀ cycloalkyl-C₁-C₁₀ alkyl unsubstituted or substituted by oxo; aryl-C₁-C₁₀ alkyl unsubstituted or substituted by one or more of halogen, C₁-C₁₀ alkoxy, halo-C₁-C₁₀ alkyl, halo-C₁-C₁₀ alkoxy, halo-C₁-C₁₀ alkylthio, C₁-C₁₀ alkylsulfonyl, oxo and heteroaryl; heteroaryl-C₁-C₁₀ alkyl unsubstituted or substituted by one or more of halogen, C₁-C₁₀ alkyl, C₁-C₁₀ alkylsulfonyl, halo-C₁-C₁₀ alkyl, oxo and aryl, wherein the aryl group is unsubstituted or substituted by halogen; heterocyclyl-C₁-C₁₀ alkyl unsubstituted or substituted by one or more of halogen, oxo and aryl.

20. The method of claim 19 wherein the functional gastrointestinal disorder is functional dyspepsia.

21. A method for the treatment of irritable bowel syndrome (IBS) comprising administering an effective amount of a compound of formula (I) or a pharmaceutically acceptable salt thereof, optionally in combination with a GABA_{B} receptor agonist, to a subject, wherein the compound of formula (I) comprises 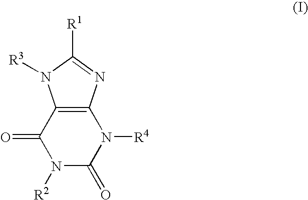
wherein
R¹ is selected from halogen; C₁-C₁₀ alkyl; C₁-C₁₀ alkoxy; hydroxy-C₁-C₁₀ alkyl; C₁-C₁₀ alkoxy-C₁-C₁₀ alkyl; C₃-C₁₀ cycloalkyl; amino substituted by one or more of C₁-C₁₀ alkyl and C₁-C₁₀ alkoxy-C₁-C₁₀ alkyl; and heterocyclyl unsubstituted or substituted by one or more of C₁-C₁₀ alkyl, C₁-C₁₀ alkoxy, C₁-C₁₀ alkoxy-C₁-C₁₀ alkyl, di-C₁-C₁₀ alkylamino, oxo and heterocyclyl-C₁-C₁₀ alkyl;
R² is selected from benzyl substituted by one or more of halogen; cyano; C₁-C₁₀ alkyl; C₁-C₁₀ alkoxy; aroyl; halo-C₁-C₁₀ alkyl; aryl-C₁-C₁₀ alkoxy and C₁-C₁₀ alkoxycarbonyl; 2-naphthylmethyl; 1-(4-chlorophenyl)-5-(trifluoromethyl)-1H-pyrazol-4-ylmethyl; 2-(4-chlorophenyl)ethyl; 2,1,3-benzothiadiazol-5-ylmethyl; and 1-[5-(trifluoromethyl)]-1,3-benzothiazol-2-ylmethyl;
R³ is selected from C₁-C₁₀ alkyl and aryl substituted by one or more of halogen; and
R⁴ is selected from ethyl; isobutyl; propyl; 3,3-dimethylbutyl; C₁-C₁₀ alkyl substituted by one or more of hydroxy, oxo, C₁-C₁₀ alkoxy, C₁-C₁₀ alkoxycarbonylamino, tri-C₁-C₁₀ alkylsilyl, tri-C₁-C₁₀ alkylsilyloxy, C₁-C₁₀ alkylsulfonyl and aryloxy, wherein the aryloxy is substituted by one or more of halo-C₁-C₁₀ alkyl; amino-C₁-C₁₀ alkyl substituted by oxo; di-C₁-C₁₀ alkylamino-C₁-C₁₀ alkyl unsubstituted or substituted by one or more of oxo; halo-C₁-C₁₀ alkyl unsubstituted or substituted by one or more of hydroxy; C₁-C₁₀ alkoxycarbonyl-C₁-C₁₀ alkyl; C₂-C₁₀ alkenyl; C₃-C₁₀ cycloalkyl-C₁-C₁₀ alkyl unsubstituted or substituted by oxo; aryl-C₁-C₁₀ alkyl unsubstituted or substituted by one or more of halogen, C₁-C₁₀ alkoxy, halo-C₁-C₁₀ alkyl, halo-C₁-C₁₀ alkoxy, halo-C₁-C₁₀ alkylthio, C₁-C₁₀ alkylsulfonyl, oxo and heteroaryl; heteroaryl-C₁-C₁₀ alkyl unsubstituted or substituted by one or more of halogen, C₁-C₁₀ alkyl, C₁-C₁₀ alkylsulfonyl, halo-C₁-C₁₀ alkyl, oxo and aryl, wherein the aryl group is unsubstituted or substituted by halogen; heterocyclyl-C₁-C₁₀ alkyl unsubstituted or substituted by one or more of halogen, oxo and aryl.

22. The method of claim 21 wherein the IBS is constipation predominant IBS.

23. The method of claim 21 wherein the IBS is diarrhea predominant IBS.

24. The method of claim 21 wherein the IBS is alternating bowel movement predominant IBS.
